(19) United States
(12) Patent Application Publication
(10) Pub. No.: US 2026/0265220 A1
(43) Pub. Date: Sep. 10, 2026

(54) GALPHA12 INHIBITORS AND METHODS USING SAME

(71) Applicants: RUTGERS, THE STATE UNIVERSITY OF NEW JERSEY, New Brunwick, NJ (US); The Trustees of Princeton University, Princeton, NJ (US)

(72) Inventors: Reynold A. PANETTIERI, New Brunswick, NJ (US); Edwin YOO, New Brunswick, NJ (US); Hahn Kim, Princeton, NJ (US)

(21) Appl. No.: 18/993,541

(22) PCT Filed: Jul. 11, 2023
(86) PCT No.: PCT/US23/69924

§ 371 Date: Jan. 13, 2025

Related U.S. Application Data
(60) Provisional application No. 63/388,013, filed on Jul. 11, 2022.

(51) Int. Cl. C07D 401/12; A61K 31/192; A61K 31/353; A61K 31/4439; A61K 31/47; A61K 31/4709; A61K 31/4745; A61K 31/496; A61K 31/497; A61K 31/506; A61K 31/519; A61K 31/52; A61K 31/5377; A61K 31/551; A61K 31/553; A61P 11/08; C07D 213/50; C07D 239/42; C07D 401/14; C07D 403/14; C07D 417/12; C07D 417/14

(52) U.S. Cl. CPC C07D 401/12; A61K 31/192; A61K 31/353; A61K 31/4439; A61K 31/47; A61K 31/4709; A61K 31/4745; A61K 31/496; A61K 31/497; A61K 31/506; A61K 31/519; A61K 31/52; A61K 31/5377; A61K 31/551; A61K 31/553; A61P 11/08; C07D 213/50; C07D 239/42; C07D 401/14; C07D 403/14; C07D 417/12; C07D 417/14

(57) ABSTRACT

The present disclosure relates to compounds, and methods of use thereof, useful for the treatment of airway disease in a subject. In one aspect, the present disclosure relates to a method of treating, preventing, and/or ameliorating an airway disease in a subject, the method comprising administering to the subject a compound of the present disclosure. In certain embodiments, bronchodilation is promoted in the subject and/or bronchoconstriction is inhibited in the subject. In certain embodiments, the airway disease is selected from the group consisting of asthma, chronic obstructive pulmonary disease (COPD), chronic bronchitis, bronchiectasis, and cystic fibrosis.

### CROSS-REFERENCE TO RELATED APPLICATIONS

This application claims priority under 35 U.S.C. § 119(e) to U.S. Provisional Patent Application No. 63/388,013, filed Jul. 11, 2022, which is incorporated herein by reference in its entirety.

### STATEMENT REGARDING FEDERALLY SPONSORED RESEARCH

This invention was made with government support under grant number HL114471-08 awarded by the National Institutes of Health. The government has certain rights in this invention.

### BACKGROUND

Respiratory and/or airway diseases, including chronic obstructive pulmonary disease (COPD), asthma, and cystic fibrosis, are among the leading causes of death and disability. Approximately 65 million people suffer from COPD, with approximately 3 million deaths annually, making COPD the third leading cause of death worldwide. Additionally, asthma is the most common chronic disease in children, affecting 14% of all children globally, or approximately 334 million people in total. It is estimated that 4 million people die prematurely from such respiratory diseases.

Chronic inflammatory respiratory diseases obstruct airflow to and from the lungs, symptoms of which may include difficulty breathing, coughing, mucus production, and/or wheezing. Symptomatic relief may be achieved by administration of bronchodilators (e.g., short-acting and/or long-acting), which relax muscles in the lungs and widen bronchi (i.e., airways). The most common bronchodilators are β₂ adrenergic agonists (e.g., salbutamol, salmeterol, formoterol, and vilanterol) and anticholinergics (e.g., ipratropium, tiotropium, aclidinium, and glycopyrronium), which act by activating β₂ adrenergic receptors and preventing the action of acetylcholine, respectively.

Alternatively, inhibition of calcium sensitization pathways in human airway smooth muscle has been shown to be an effective method of inducing bronchodilation. However, due to off target effects in vascular smooth muscle cells, clinical trials of bronchodilators targeting calcium sensitization pathways have been precluded.

There is thus a need in the art for compositions and methods of treating airway disease. The present disclosure addresses this need.

### BRIEF SUMMARY

In one aspect, the present disclosure provides a method of treating, preventing, and/or ameliorating an airway disease in a subject. In certain embodiments, the method comprises administering to the subject a compound of formula (I), or a salt, solvate, prodrug, stereoisomer, isotopologue, or tautomer thereof, or any mixtures thereof, wherein A¹, A², B, L¹, L², R^{1a}, R^{1b}, R^{2a}, R^{2b}, R^{3a}, R^{3b}, X, Y¹, and Y² are defined elsewhere herein:

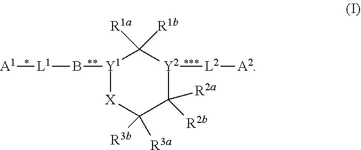

In certain embodiments, the airway disease is selected from the group consisting of asthma, chronic obstructive pulmonary disease (COPD), chronic bronchitis, bronchiectasis, and cystic fibrosis.

In another aspect, the present disclosure provides a method of inhibiting bronchoconstriction and/or promoting bronchodilation in a subject. In certain embodiments, the method comprises administering to the subject a compound of formula (I), or a salt, solvate, prodrug, stereoisomer, isotopologue, or tautomer thereof, or any mixtures thereof, wherein A¹, A², B, L¹, L², R^{1a}, R^{1b}, R^{2a}, R^{2b}, R^{3a}, R^{3b}, X, Y¹, and Y² are defined elsewhere herein:

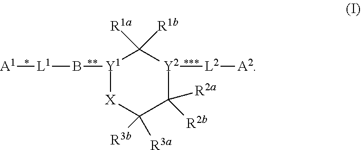

In another aspect, the present disclosure provides a compound of formula (II), or a salt, solvate, prodrug, stereoisomer, isotopologue, or tautomer thereof, or any mixtures thereof, wherein A¹, A², B, L¹, L², R^{1a}, R^{1b}, R^{2a}, R^{2b}, R^{3a}, R^{3b}, X, Y¹, and Y² are defined elsewhere herein:

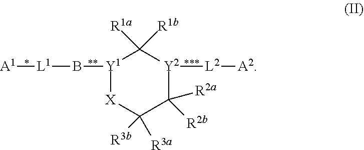

### BRIEF DESCRIPTION OF THE FIGURES

The drawings illustrate generally, by way of example, but not by way of limitation, various embodiments of the present application.

FIGS. 1A-1C show an experimental approach to the identification of Gα₁₂ modulators using hTERT-SRE human telomerase reverse transcriptase immortalized airway smooth muscle cells. FIG. 1A: provides a schematic representation of Gα₁₂-activated luciferase reporter. FIG. 1B: shows the overall reaction underlying the luciferase reporter assay. FIG. 1C: provides a detailed schematic showing the downstream effects of Gα_{12/13} activation and/or inhibition, wherein serum response element (SRE) may be detected by a luciferase assay, and pAKT and pMLC may be detected by Western blotting.

FIG. 2 provides a bar graph showing carbachol (CCh) stimulated Luciferase Assay dose response for compound 1 (BDF) as a Gα₁₂ antagonist and BDH as a Gα₁₂ agonist; AB (aclidinium bromide).

FIGS. 3A-3B provides Z-scores of kinase activity inhibition as a function of compound concentration for selected compounds, including compounds 2 (box C), 47 (box D), 48 (box E), 49 (box F), 50 (box G), 51 (box H), 3 (box I), 52 (box J), 53 (box K), 54 (box L), 55 (box M), and 56 (box N).

FIGS. 4A-4D show that both pAKT (FIGS. 4A-4B) and pMLC (FIGS. 4C-4D) are decreased with administration of compounds 1-3, wherein a decrease in both kinases is observed with compound 1 at 100 μM and compound 3 at 10 μM and 100 μM. Additionally, a statistically significant decrease in pAKT and pMLC was observed with administration of compound 3.

FIGS. 5A-5D provide bar graphs depicting fold change in pMLC/MLC (FIG. 5A and FIG. 5C) and fold change in pAKT/AKT (FIG. 5B and FIG. 5D) induced by contractile agonists carbachol or histamine with administration of non-limiting exemplary compounds (i.e., compounds 1-3) at various concentrations.

FIGS. 6A-6B provide bar graphs depicting fold change in pMLC/MLC (FIG. 6A) and fold change in pAKT/AKT (FIG. 6B) induced by contractile agonists carbachol or histamine with administration of compound 4 at various concentrations.

FIGS. 7A-7B provide graphs which depict the efficacy and potency of certain non-limiting analogues of compound 1 for inhibiting carbachol (CCh) activation of pMLC (FIG. 7A) and pAKT (FIG. 7B).

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “about” as used herein can allow for a degree of variability in a value or range, for example, within 10%, within 5%, or within 1% of a stated value or of a stated limit of a range, and includes the exact stated value or range.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C═CH, —C═C(CH₃), —C═C(CH₂CH₃), —CH₂C═CH, —CH₂C═C(CH₃), and —CH₂C═C(CH₂CH₃) among others.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “atm” as used herein refers to a pressure in atmospheres under standard conditions. Thus, 1 atm is a pressure of 101 kPa, 2 atm is a pressure of 202 kPa, and so on.

The term “BDH” as used herein refers to 2-(2-fluorophenyl)-N-(1-(6-(isoquinolin-8-ylmethyl)-1,4-oxazepan-4-yl)-2-methyl-1-oxopropan-2-yl)acetamide:

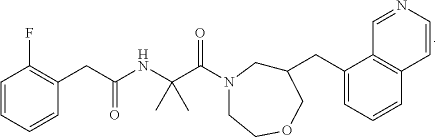 [SMILES: CC(C)(NC(=O)Cc1ccccc1F)C(=O)N1CCOCC(Cc2cccc3ccncc23)C1]

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system.

An appropriate therapeutic amount in any individual case may be determined by one of ordinary skill in the art using routine experimentation.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxy cyclohexyl)ethyl, 2-(2,3-epoxy cylopentyl)ethyl, 2-(4-methyl-3,4-epoxycyclohexyl)propyl, 2-(3,4-epoxy-3-methylcylohexyl)-2-methylethyl, and 5,6-epoxyhexyl.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl. N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “monovalent” as used herein refers to a substituent connecting via a single bond to a substituted molecule. When a substituent is monovalent, such as, for example, F or Cl, it is bonded to the atom it is substituting by a single bond.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “room temperature” as used herein refers to a temperature of about 15° C. to 28° C.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate).

Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, Q-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The term “solvent” as used herein refers to a liquid that can dissolve a solid, liquid, or gas. Non-limiting examples of solvents are silicones, organic compounds, water, alcohols, ionic liquids, and supercritical fluids.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The term “thioalkyl” as used herein refers to a sulfur atom connected to an alkyl group, as defined herein. The alkyl group in the thioalkyl can be straight chained or branched. Examples of linear thioalkyl groups include but are not limited to thiomethyl, thioethyl, thiopropyl, thiobutyl, thiopentyl, thiohexyl, and the like. Examples of branched alkoxy include but are not limited to iso-thiopropyl, sec-thiobutyl, tert-thiobutyl, iso-thiopentyl, iso-thiohexyl, and the like. The sulfur atom can appear at any suitable position in the alkyl chain, such as at the terminus of the alkyl chain or anywhere within the alkyl chain.

The terms “treat,” “treating” and “treatment,” as used herein, means reducing the frequency or severity with which symptoms of a disease or condition are experienced by a subject by virtue of administering an agent or compound to the subject.

### Methods

In one aspect, the present disclosure provides a method of treating, preventing, and/or ameliorating an airway disease in a subject.

In certain embodiments, contraction of an airway smooth muscle cell is inhibited. In certain embodiments, dilation of an airway smooth muscle cell is promoted.

In certain embodiments, the airway disease is at least one selected from the group consisting of asthma, chronic obstructive pulmonary disease (COPD), chronic bronchitis, bronchiectasis, and cystic fibrosis.

In another aspect, the present disclosure provides a method of inhibiting bronchoconstriction and/or promoting bronchodilation in a subject.

In certain embodiments, Gα₁₂ (also referred to as Galpha₁₂) is inhibited. In certain embodiments, the method further comprises administering to the subject a therapeutically effective amount of one or more additional agents. In certain embodiments, the one or more additional agents are selected from the group consisting of a phosphoinositide 3-kinase (PI3K) inhibitor and a rho kinase (ROCK) inhibitor.

In certain embodiments, the PI3K inhibitor is at least one selected from the group consisting of idelalisib, copanlisib, duvelisib, alpelisib, umbralisib, buparlisib, dactolisib, leniolisib, parsaclisib, paxalisib, taselisib, zandelisib, inavolisib, apitolisib, bimarlisib, eganelisib, fimepinostat, gedatolisib, linperlisib, nemiralisib, pictilisib, pilaralisib, samotolisib, seletalisib, serabelisib, sonolisib, tenalisib, voxtalisib, AMG 319, AZD8186, GSK2636771, SF1126, acalisib, omipalisib, AZD8835, CAL263, GSK1059615, MEN1611, PWT33597, TG100-115, and ZSTK474.

In certain embodiments, the ROCK inhibitor is at least one selected from the group consisting of rhosin, AT-13148, BA-210, β-elemene, chroman 1, DJ4, fasudil, GSK-576371, GSK429286A, H-1152, hydroxyfasudil, ibuprofen, LX-7101, netarsudil, RKI-1447, ripasudil, TCS-7001, thiazovivin, verosudil, Y-27632, Y-30141, Y-33075, and Y-39983.

In certain embodiments, the subject is administered a compound of formula (I):

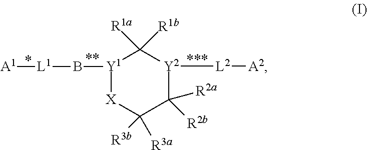

wherein:
A¹ is selected from the group consisting of optionally substituted phenyl and optionally substituted C₁-C₁₀ heterocyclyl;
A² is selected from the group consisting of optionally substituted phenyl, optionally substituted C₁-C₁₀ heterocyclyl, and NH (optionally substituted C₂-C₈ heterocyclyl);
B is

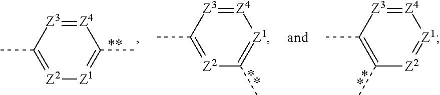

L¹ is selected from the group consisting of a bond, —NR¹—, —O—, and optionally substituted C₁-C₃ alkylenyl;
L² is selected from the group consisting of a bond, —C(═O)—, —C(═O)O—, —C(═O)(optionally substituted C₁-C₃ alkylenyl)-, and optionally substituted C₁-C₃ alkylenyl;
X is selected from the group consisting of —O— and —C(R^{5a})(R^{5b})—,
Y¹ and Y² are each independently selected from the group consisting of N and CR⁶,
   wherein no more than one of Y¹ and Y² is N, and
   wherein if X is 0, then Y¹ is CR⁶;
Z¹ is selected from the group consisting of CR⁷a and N, Z² is selected from the group consisting of CR^{7b} and N, Z³ is selected from the group consisting of CR^{7c} and N, and Z⁴ is selected from the group consisting of CR^{7d} and N,
   wherein 0-2 selected from the group consisting of Z¹, Z², Z³, and Z⁴ are N;
R^{1a}, R^{1b}, R^{2a}, R^{2b}, R^{3a}, and R^{3b} are each independently selected from the group consisting of H, optionally substituted C₁-C₆ alkyl, optionally substituted C₃-C₈ cycloalkyl, optionally substituted C₁-C₆ alkoxy, and halogen;
R⁴ is selected from the group consisting of H and optionally substituted C₁-C₆ alkyl;
R^{5a} and R^{5b} are each independently selected from the group consisting of H, optionally substituted C₁-C₆ alkyl, optionally substituted C₃-C₈ cycloalkyl, optionally substituted C₁-C₆ alkoxy, and halogen;
R⁶ is selected from the group consisting of H, optionally substituted C₁-C₆ alkyl, optionally substituted C₃-C₈ cycloalkyl, optionally substituted C₁-C₆ alkoxy, and halogen;
R^{7a}, R^{7b}, R^{7c}, and R^{7d} are each independently selected from the group consisting of H, optionally substituted C₁-C₆ alkyl, optionally substituted C₃-C₈ cycloalkyl, optionally substituted C₁-C₆ alkoxy, optionally substituted phenyl, optionally substituted C₂-C₈ heterocyclyl, halogen, CN, NO₂, OR^{a}, N(R^{a})(R^{b}), C(═O)R^{a}, C(═O)OR^{a}, OC(═O)R^{a}, C(═O)N(R^{a})(R^{b}), S(═O)₂N(R^{a})(R^{b}), NR^{a}C(═O)R^{b}, and NR^{a}S(═O)₂R^{b};
   wherein two vicinal substituents selected from the group consisting of R^{7a}, R^{1b}, R^{7c}, and R^{7d} can combine with the atoms to which they are bound to form an optionally substituted phenyl or optionally substituted C₂-C₈ heterocyclyl;
R^{a} and R^{b} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl;
* indicates a bond between A¹ and L¹;
** indicates a bond between B and Y¹; and
*** indicates a bond between Y² and L²1
or a salt, solvate, prodrug, stereoisomer, isotopologue, or tautomer thereof, or any mixtures thereof.

In certain embodiments, the compound of formula (I) is selected from the group consisting of:

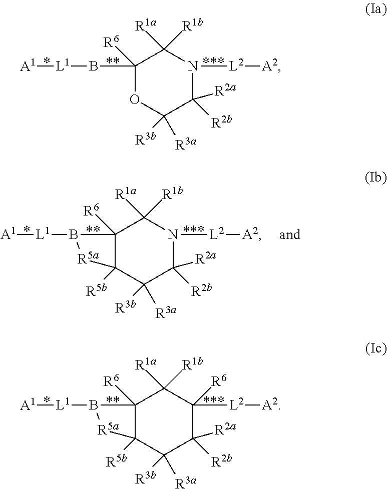

In certain embodiments, the compound of formula (I) is selected from the group consisting of:

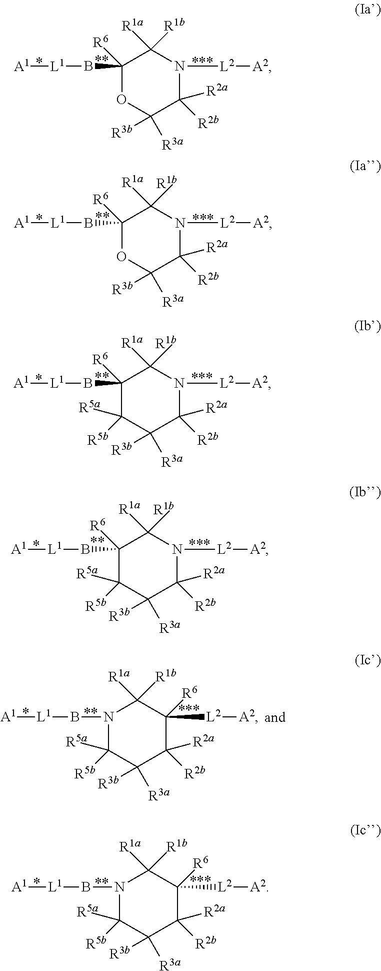

In certain embodiments, R^{1a}, R^{1b}, R^{2a}, R^{2b}, R^{3a}, and R^{3b} are each independently H. In certain embodiments, R^{1a}, R^{1b}, R^{2a}, R^{2b}, R^{3a}, R^{3b}, R^{5a}, and R^{5b} are each independently H. In certain embodiments, R^{1a}, R^{1b}, R^{2a}, R^{2b}, R^{3a}, R^{1b}, and R⁶ are each independently H. In certain embodiments, R^{1a}, R^{1b}, R^{2a}, R^{2b}, R^{3a}, R^{3b}, R^{5a}, R^{5b}, and R⁶ are each independently H.

In certain embodiments, B is

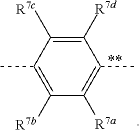

In certain embodiments, B is

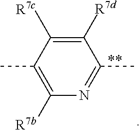

In certain embodiments, B is

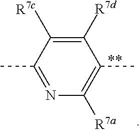

In certain embodiments, B is

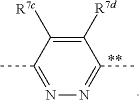

In certain embodiments, B is

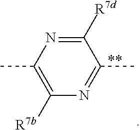

In certain embodiments, B is

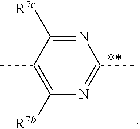

In certain embodiments, B is

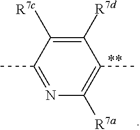

In certain embodiments, B is

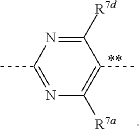

In certain embodiments, B is

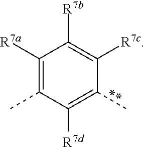

In certain embodiments, B is

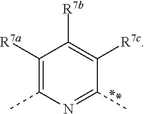

In certain embodiments, B is

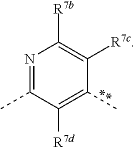

In certain embodiments, B is

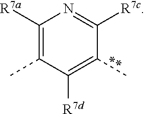

In certain embodiments, B is

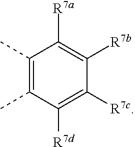

In certain embodiments, B is

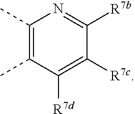

In certain embodiments, B is

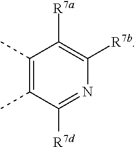

In certain embodiments, at least one selected from the group consisting of R^{7a}, R^{7b}, R^{7c}, and R^{7d} is H. In certain embodiments, at least two selected from the group consisting of R^{7a}, R^{1b}, R^{7c}, and R^{7d} are H. In certain embodiments, at least three selected from the group consisting of R^{7a}, R^{7b}, R^{7c}, and R^{7d} are H. In certain embodiments, each of R^{7a}, R^{7b}, R^{7c}, and R^{7d} are H. In certain embodiments, two vicinal substituents selected from the group consisting of R^{7a}, R^{7b}, R^{7c}, and R^{7d} combine to form phenyl.

In certain embodiments, B is

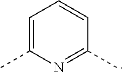

In certain embodiments, B is

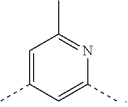

In certain embodiments, B is

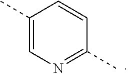

In certain embodiments, B is

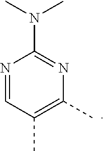

In certain embodiments, B is

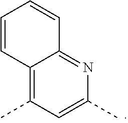

In certain embodiments, the phenyl in A¹ is optionally substituted with at least one substituent selected from the group consisting of methyl, trifluoromethyl, fluorine, chlorine, methoxy, and C(═O)NH₂. In certain embodiments, the heterocyclyl in A¹ is optionally substituted with at least one substituent selected from the group consisting of methyl, trifluoromethyl, fluorine, chlorine, methoxy, and C(═O)NH₂.

In certain embodiments, A¹ is

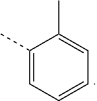

In certain embodiments, A¹ is

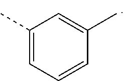

In certain embodiments, A¹ is

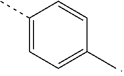

In certain embodiments, A¹ is

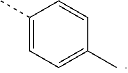

In certain embodiments, A¹ is

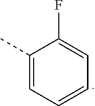

In certain embodiments, A¹ is

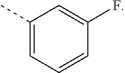

In certain embodiments, A¹ is

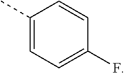

In certain embodiments, A¹ is

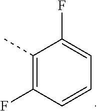

In certain embodiments, A¹ is

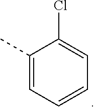

In certain embodiments, A¹ is

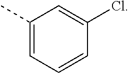

In certain embodiments, A¹ is

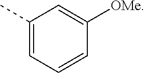

In certain embodiments, A¹ is

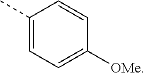

In certain embodiments, A¹ is

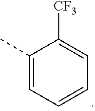

In certain embodiments, A¹ is

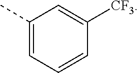

In certain embodiments, A¹ is

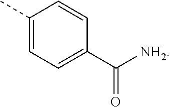

In certain embodiments, A¹ is

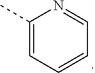

In certain embodiments, A¹ is

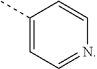

In certain embodiments, A¹ is

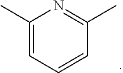

In certain embodiments, A¹ is

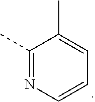

In certain embodiments, A¹ is

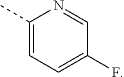

In certain embodiments, A¹ is

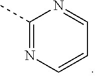

In certain embodiments, A¹ is

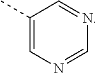

In certain embodiments, A¹ is

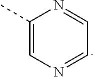

In certain embodiments, A¹ is

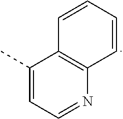

In certain embodiments, A¹ is

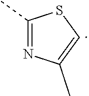

In certain embodiments, A¹ is

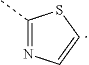

In certain embodiments, A¹ is

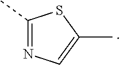

In certain embodiments, A¹ is

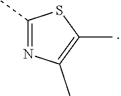

In certain embodiments, A¹ is

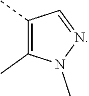

In certain embodiments, A¹ is

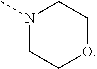

In certain embodiments, A² is selected from the group consisting of

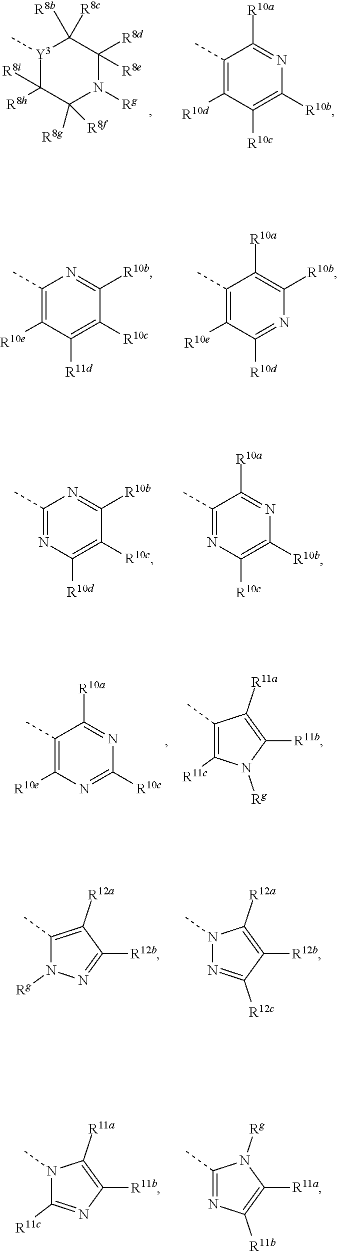

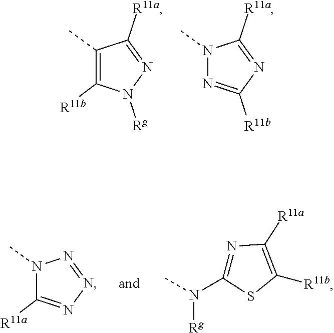

wherein:
R^{8a}, R^{8b}, R^{8c}, R^{8d}, R^{8e}, R^{8f}, R^{8g}, R^{8h}, and R⁸ⁱ are each independently selected from the group consisting of H, optionally substituted C₁-C₆ alkyl, optionally substituted C₃-C₈ cycloalkyl, and halogen,
   wherein any two substituents selected from the group consisting of R^{8a}, R^{8b}, R^{8c}, R^{8d}, R^{8e}, R^{8f}, R^{8g}, R^{8h}, and R⁸ⁱ may combine with the atoms to which they are bound to form an optionally substituted C₃-C₈ cycloalkyl or optionally substituted C₂-C₈ heterocyclyl;
R⁹ is selected from the group consisting of H, optionally substituted C₁-C₆ alkyl, optionally substituted C₁-C₆ cycloalkyl, and optionally substituted benzyl;
R^{10a}, R^{10b}, R^{10c}, R^{10d}, and R^{10c}, if present, are each independently selected from the group consisting of H, optionally substituted C₁-C₆ alkyl, optionally substituted C₃-C₈ cycloalkyl, halogen, N(R^{a})(R^{b}), CN, and NO₂,
   wherein no more than one of R^{10a}, R^{10b}, R^{10c}, R^{10d} and R^{10e} is N(R^{a})(R^{b}), and two vicinal substituents selected from the group consisting of R^{10a}, R^{10b}, R^{10c}, R^{10d}, and R^{10c} can combine with the atoms to which they are bound to form an optionally substituted phenyl or optionally substituted C₂-C₈ heterocyclyl;
R^{11e}, R^{11b}, and R^{11c}, if present, are each independently selected from the group consisting of H, optionally substituted C₁-C₆ alkyl, optionally substituted C₃-C₈ cycloalkyl, halogen, CN, and NO₂,
   wherein two vicinal substituents selected from the group consisting of R^{11a}, R^{11b}, and R^{11c} can combine with the atoms to which they are bound to form an optionally substituted phenyl or optionally substituted C₂-C₈ heterocyclyl; and
Y³ is selected from the group consisting of R^{8a} and N.

In certain embodiments, A² is

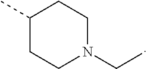

In certain embodiments, A² is

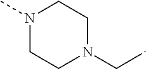

In certain embodiments, A² is

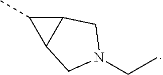

In certain embodiments, A² is

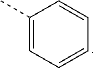

In certain embodiments, A² is

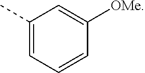

In certain embodiments, A² is

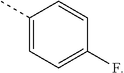

In certain embodiments, A² is

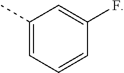

In certain embodiments, A² is

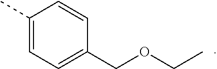

In certain embodiments, A² is

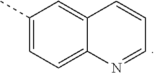

In certain embodiments, A² is

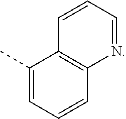

In certain embodiments, A² is

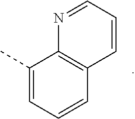

In certain embodiments, A² is

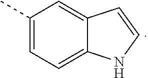

In certain embodiments, A² is

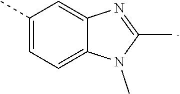

In certain embodiments, A² is

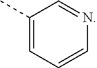

In certain embodiments, A² is

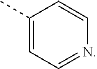

In certain embodiments, A² is

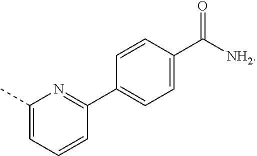

In certain embodiments, A² is

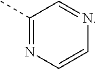

In certain embodiments, A² is

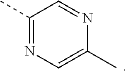

In certain embodiments, A² is

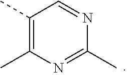

In certain embodiments, A² is

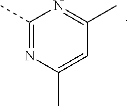

In certain embodiments, A² is

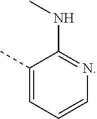

In certain embodiments, A² is

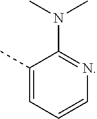

In certain embodiments, A² is

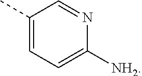

In certain embodiments, A² is

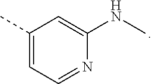

In certain embodiments, A² is

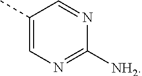

In certain embodiments, A² is

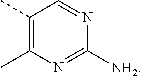

In certain embodiments, A² is

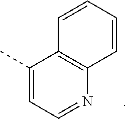

In certain embodiments, A² is

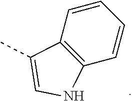

In certain embodiments, A² is

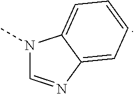

In certain embodiments, A² is

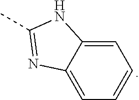

In certain embodiments, A² is

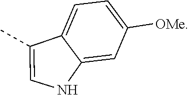

In certain embodiments, A² is

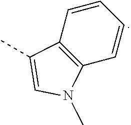

In certain embodiments, A² is

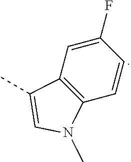

In certain embodiments, A² is

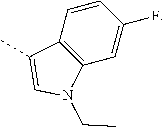

In certain embodiments, A² is

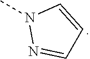

In certain embodiments, A² is

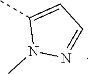

In certain embodiments, A² is

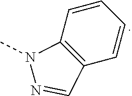

In certain embodiments, A² is

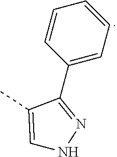

In certain embodiments, A² is

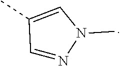

In certain embodiments, A² is

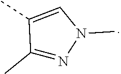

In certain embodiments, A² is

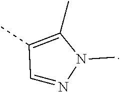

In certain embodiments, A² is

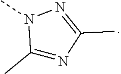

In certain embodiments, A² is

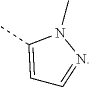

In certain embodiments, A² is

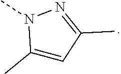

In certain embodiments, A² is

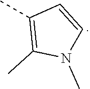

In certain embodiments, A² is

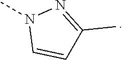

In certain embodiments, A² is

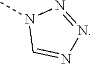

In certain embodiments, A² is

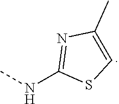

In certain embodiments, A² is

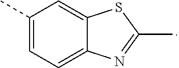

In certain embodiments, L¹ is a bond. In certain embodiments, L¹ is —NH—. In certain embodiments, L¹ is —O—. In certain embodiments, L¹ is —CH₂—.

In certain embodiments, L² is a bond. In certain embodiments, L² is —CH₂—. In certain embodiments, L² is —CH₂CH₂—. In certain embodiments, L² is —CH₂CH₂CH₂—. In certain embodiments, L² is —C(═O)—. In certain embodiments, L² is

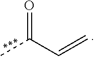

In certain embodiments, L² is

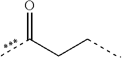

In certain embodiments, each occurrence of optionally substituted alkyl, optionally substituted cycloalkyl, optionally substituted heterocyclyl, optionally substituted alkylenyl, optionally substituted alkoxy, optionally substituted benzyl, and optionally substituted phenyl, if present, is optionally substituted with at least one substituent selected from the group consisting of C₁-C₆ alkyl, C₁-C₆ heteroalkyl, C₃-C₈ cycloalkyl, C₁-C₆ haloalkyl, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, wherein each occurrence of R′ and R″ is independently selected from the group consisting of H, C₁-C₆ alkyl, C₃-C₈ cycloalkyl, C₁-C₆ haloalkyl, benzyl, and phenyl.

In certain embodiments, the compound of formula (I) is selected from the group consisting of:
1-(2-(5-(4-fluorobenzyl)pyridin-2-yl)morpholino)-3-(1H-indazol-1-yl)propan-1-one;
2-(1-ethylpiperidin-4-yl)-1-(2-(5-(3-methoxyphenyl)pyridin-2-yl)morpholino)ethan-1-one;
(2-(5-(4-methylbenzyl)pyridin-2-yl)morpholino)(5-methylpyrazin-2-yl)methanone;
N-methyl-3-((2-(5-(3-(trifluoromethyl)benzyl)pyridin-2-yl)morpholino)methyl)pyridin-2-amine;
4-(pyridin-3-ylmethyl)-2-(5-(3-(trifluoromethyl)benzyl)pyridin-2-yl)morpholine;
2-(6-(4-fluorobenzyl)pyridin-2-yl)-4-(quinolin-6-ylmethyl)morpholine;
4-(6-(4-(quinolin-4-ylmethyl)morpholin-2-yl)pyridin-2-yl)benzamide;
morpholino(2-(4-phenethylmorpholin-2-yl)quinolin-4-yl)methanone;
(2-(4-(3-methoxybenzyl)morpholin-2-yl)quinolin-4-yl)(morpholino)methanone;
2-(4-(2-(3,4-dimethylphenyl)acetyl)morpholin-2-yl)-N-(2-hydroxyethyl)quinoline-4-carboxamide;
4-(6-(4-(4-(4-fluorophenyl)butanoyl)morpholin-2-yl)pyridin-3-yl)benzamide;
3-(1H-indol-3-yl)-1-(2-(6-(4-methoxyphenyl)pyridin-2-yl)morpholino)propan-1-one;
N-(2-hydroxyethyl)-2-(4-((1-methyl-1H-indol-3-yl)methyl)morpholin-2-yl)quinoline-4-carboxamide;
N-(2-methoxyethyl)-2-(4-phenethylmorpholin-2-yl)quinoline-4-carboxamide;
3-(1H-benzo[d]imidazol-2-yl)-1-(2-(6-(2-fluorobenzyl)pyridin-2-yl)morpholino)propan-1-one;
3-(1H-benzo[d]imidazol-2-yl)-1-(2-(5-(2-fluorobenzyl)pyridin-2-yl)morpholino)propan-1-one;
(2-(6-(2-chlorobenzyl)pyridin-2-yl)morpholino)(quinolin-5-yl)methanone;
(2-(6-benzylpyridin-2-yl)morpholino)(quinolin-8-yl)methanone;
2-(5-(4-methoxybenzyl)pyridin-2-yl)-4-(quinolin-5-ylmethyl)morpholine;
2-(4-benzylmorpholin-2-yl)-N-(thiazol-2-yl)quinoline-4-carboxamide;
(2-(5-(2-chlorobenzyl)pyridin-2-yl)morpholino)(quinolin-8-yl)methanone;
2-(4-((1H-indol-5-yl)methyl)morpholin-2-yl)-N-(2-hydroxyethyl)-N-methylquinoline-4-carboxamide;
2-(4-benzoylmorpholin-2-yl)-N-isobutylquinoline-4-carboxamide;
5-(4-fluorophenyl)-N,N-dimethyl-4-(4-(quinolin-4-ylmethyl)morpholin-2-yl)pyrimidin-2-amine;
1-(2-(2-(dimethylamino)-5-(3-fluorophenyl)pyrimidin-4-yl)morpholino)-2-(4-fluorophenyl)ethan-1-one;
N-isopropyl-2-(4-(quinolin-4-ylmethyl)morpholin-2-yl)quinoline-4-carboxamide;
N-methyl-2-(4-((3-phenyl-1H-pyrazol-4-yl)methyl)morpholin-2-yl)quinoline-4-carboxamide;
1-(2-(6-(2,6-difluorophenyl)pyridin-2-yl)morpholino)-3-(1H-indol-3-yl)propan-1-one;
(2-(4-benzylmorpholin-2-yl)quinolin-4-yl)(morpholino)methanone;
2-(4-(2-(4-fluorophenyl)acetyl)morpholin-2-yl)-N-isobutylquinoline-4-carboxamide;
2-(4-((1H-indol-5-yl)methyl)morpholin-2-yl)-N-(2-methoxyethyl)quinoline-4-carboxamide;
N-(2-hydroxyethyl)-2-(4-(quinolin-6-ylmethyl)morpholin-2-yl)quinoline-4-carboxamide;
phenyl(2-(5-(3-(trifluoromethyl)benzyl)pyridin-2-yl)morpholino)methanone;
1-(2-(5-(2-fluorobenzyl)pyridin-2-yl)morpholino)-3-(1H-indol-3-yl)propan-1-one;
2-(5-(4-methoxybenzyl)pyridin-2-yl)-4-(quinolin-4-ylmethyl)morpholine;
1-(2-(6-(2-fluorobenzyl)pyridin-2-yl)morpholino)-2-(3-fluorophenyl)-2-methylpropan-1-one;
N-isobutyl-2-(4-(2-phenylacetyl)morpholin-2-yl)quinoline-4-carboxamide;
1-(2-(4-(3-fluorobenzyl)-6-methylpyridin-2-yl)morpholino)-2-(4-fluorophenyl)ethan-1-one;
3-(6-(4-(4,6-dimethylpyrimidin-2-yl)morpholin-2-yl)pyridin-3-yl)propenamide;
4-((1,5-dimethyl-1H-pyrazol-4-yl)methyl)-2-(5-(pyrimidin-5-yl)pyridin-2-yl)morpholine;
(2-(6-(2-fluorobenzyl)pyridin-2-yl)morpholino)(2-(methylamino)pyridin-4-yl)methanone;
N-(6-(4-(quinolin-5-ylmethyl)morpholin-2-yl)pyridin-2-yl)pyrazin-2-amine;
N-(6-(4-((6-methoxy-1-methyl-1H-indol-3-yl)methyl)morpholin-2-yl)pyridin-2-yl)-4-methylthiazol-2-amine;
N-(6-(4-((2-(dimethylamino)pyridin-3-yl)methyl)morpholin-2-yl)pyridin-2-yl)-4-methylthiazol-2-amine;
4-methyl-N-(6-(4-(3-methylbenzyl)morpholin-2-yl)pyridin-2-yl)thiazol-2-amine;
2-(6-(4-methoxyphenyl)pyridin-2-yl)-4-(quinolin-5-ylmethyl)morpholine;
2-(6-(2-fluorobenzyl)pyridin-2-yl)-4-(quinolin-5-ylmethyl)morpholine;
2-(1H-benzo[d]imidazol-1-yl)-1-(2-(4-(2-fluorobenzyl)-6-methylpyridin-2-yl)morpholino)ethan-1-one;
morpholino(2-(4-(3-phenylpropyl)morpholin-2-yl)quinolin-4-yl)methanone;
(2-(6-(3-methoxybenzyl)pyridin-2-yl)morpholino)(quinolin-8-yl)methanone;
3-(3-((2-(6-(3-fluorophenyl)pyridin-2-yl)morpholino)methyl)-1H-indol-1-yl)propan-1-ol;
3-(1H-benzo[d]imidazol-1-yl)-1-(2-(6-(4-fluorobenzyl)pyridin-2-yl)morpholino)propan-1-one;
2-(1H-indol-3-yl)-1-(2-(6-(3-methoxybenzyl)pyridin-2-yl)morpholino)ethan-1-one;
N-(6-(4-(quinolin-4-ylmethyl)morpholin-2-yl)pyridin-2-yl)pyrazin-2-amine;
5-fluoro-N-(6-(4-((6-methoxy-1-methyl-1H-indol-3-yl)methyl)morpholin-2-yl)pyridin-2-yl)pyridin-2-amine;
N-benzyl-N-isopropyl-2-(2-(6-(pyridin-2-ylamino)pyridin-2-yl)morpholino)acetamide;
(2-(6-methyl-4-((6-methylpyridin-2-yl)amino)pyridin-2-yl)morpholino)(quinolin-8-yl)methanone;
1-(2-(6-(2-fluorobenzyl)pyridin-2-yl)morpholino)-3-phenylpropan-1-one;
1-(3-(4-(2-fluorobenzyl)-6-methylpyridin-2-yl)piperidin-1-yl)-3-(3-methyl-1H-pyrazol-1-yl)propan-1-one;
2-phenyl-1-(2-(6-(2-(trifluoromethyl)benzyl)pyridin-2-yl)morpholino)ethan-1-one;
N-benzyl-N-isopropyl-2-(2-(6-(pyrimidin-2-ylamino)pyridin-2-yl)morpholino)acetamide;
2′-(4-(2,4-dimethylpyrimidine-5-carbonyl)morpholin-2-yl)-N,6′-dimethyl-[3,4′-bipyridine]-6-carboxamide;
(2-(4-isonicotinoylmorpholin-2-yl)quinolin-4-yl)(morpholino)methanone;
pyridin-4-yl(2-(5-(3-(trifluoromethyl)benzyl)pyridin-2-yl)morpholino)methanone;
2-phenyl-1-(2-(5-(3-(trifluoromethyl)benzyl)pyridin-2-yl)morpholino)ethan-1-one;
(4-fluorophenyl)(2-(5-(3-(trifluoromethyl)benzyl)pyridin-2-yl)morpholino)methanone;
5-fluoro-N-(6-(4-((1-methyl-1H-indol-3-yl)methyl)morpholin-2-yl)pyridin-2-yl)pyridin-2-amine;
N-(6-(4-((1-ethyl-6-fluoro-1H-indol-3-yl)methyl)morpholin-2-yl)pyridin-2-yl)pyrazin-2-amine;
3-(1H-indazol-1-yl)-1-(2-(6-((4-methylthiazol-2-yl)amino)pyridin-2-yl)morpholino)propan-1-one;
N-(6-(4-((5-fluoro-1-methyl-1H-indol-3-yl)methyl)morpholin-2-yl)pyridin-2-yl)-4-methylthiazol-2-amine;
5-methyl-N-(2-methyl-6-(4-(quinolin-6-ylmethyl)morpholin-2-yl)pyridin-4-yl)thiazol-2-amine;
4-((1,5-dimethyl-1H-pyrazol-4-yl)methyl)-2-(6-(4-fluorobenzyl)pyridin-2-yl)morpholine;
1-(2-(6-benzylpyridin-2-yl)morpholino)-3-(1H-tetrazol-1-yl)propan-1-one;
4-((1,3-dimethyl-1H-pyrazol-4-yl)methyl)-2-(6-(2-fluorobenzyl)pyridin-2-yl)morpholine;
1-(2-(6-benzylpyridin-2-yl)morpholino)-3-(4-fluorophenyl)propan-1-one;
3-(3,5-dimethyl-1H-1,2,4-triazol-1-yl)-1-(2-(4-(4-fluorobenzyl)-6-methylpyridin-2-yl)morpholino)propan-1-one;
1-(2-(6-methyl-4-(3-(trifluoromethyl)benzyl)pyridin-2-yl)morpholino)-2-(1H-pyrazol-1-yl)ethan-1-one;
1-(2-(6-benzylpyridin-2-yl)morpholino)-2-(4-fluorophenyl)ethan-1-one;
2-(3,5-dimethyl-1H-pyrazol-1-yl)-1-(2-(6-methyl-4-(2-methylbenzyl)pyridin-2-yl)morpholino)ethan-1-one;
(6-aminopyridin-3-yl)(2-(4-(2-fluorobenzyl)-6-methylpyridin-2-yl)morpholino)methanone;
1-(2-(6-(3-chlorobenzyl)pyridin-2-yl)morpholino)-2-(4-fluorophenyl)ethan-1-one;
4-((1,5-dimethyl-1H-pyrazol-4-yl)methyl)-2-(6-(2-methylbenzyl)pyridin-2-yl)morpholine;
2-(4-benzyl-6-methylpyridin-2-yl)-4-((1,3-dimethyl-1H-pyrazol-4-yl)methyl)morpholine;
N,N-dimethyl-5-((2-(6-methyl-4-(4-methylbenzyl)pyridin-2-yl)morpholino)methyl)pyrimidin-2-amine;
N,N-dimethyl-5-((2-(6-methyl-4-(4-methylbenzyl)pyridin-2-yl)morpholino)methyl)pyrimidin-2-amine;
(1,2-dimethyl-1H-benzo[d]imidazol-5-yl)(2-(6-methyl-4-(pyridin-2-ylamino)pyridin-2-yl)morpholino)methanone;
3-(3,5-dimethyl-1H-1,2,4-triazol-1-yl)-1-(2-(6-(4-fluorobenzyl)pyridin-2-yl)morpholino)propan-1-one;
2-(4-(4-fluorobenzyl)-6-methylpyridin-2-yl)-4-((1-methyl-1H-pyrazol-4-yl)methyl)morpholine;
1-(2-(6-(2,6-difluorobenzyl)pyridin-2-yl)morpholino)-2-morpholinoethan-1-one;
(2-(6-(2,6-difluorobenzyl)pyridin-2-yl)morpholino)(2-(methylamino)pyridin-4-yl)methanone;
1-(2-(5-(3-chlorobenzyl)pyridin-2-yl)morpholino)-3-(3,5-dimethyl-1H-1,2,4-triazol-1-yl)propan-1-one;
3-((2-(6-(4-methoxyphenyl)pyridin-2-yl)morpholino)methyl)-N,N-dimethylpyridin-2-amine;
1-(2-(6-benzylpyridin-2-yl)morpholino)-3-phenylpropan-1-one;
(2-(dimethylamino)-4-methylpyrimidin-5-yl)(2-(6-methyl-4-(4-methylbenzyl)pyridin-2-yl)morpholino)methanone;
(2-(6-methyl-4-(3-(trifluoromethyl)benzyl)pyridin-2-yl)morpholino)(pyridin-3-yl)methanone;
(4-(ethoxymethyl)phenyl)(2-(6-methyl-4-((3-methylpyridin-2-yl)amino)pyridin-2-yl)morpholino)methanone;
5-((2-(6-(2-chlorobenzyl)pyridin-2-yl)morpholino)methyl)-N,N-dimethylpyrimidin-2-amine;
2-(1-ethylpiperidin-4-yl)-1-(2-(3′-methoxy-[1,1′-biphenyl]-4-yl)morpholino)ethan-1-one;
2-(1-ethylpiperidin-4-yl)-1-(3-(5-(3-methoxyphenyl)pyridin-2-yl)piperidin-1-yl)ethan-1-one;
2-(1-ethylpiperidin-4-yl)-1-(1-(5-(3-methoxyphenyl)pyridin-2-yl)piperidin-3-yl)ethan-1-one;
1-ethylpiperidin-4-yl 2-(5-(3-methoxyphenyl)pyridin-2-yl)morpholine-4-carboxylate;
4-(2-(1-ethylpiperidin-4-yl)ethyl)-2-(5-(3-methoxyphenyl)pyridin-2-yl)morpholine;
2-(1-ethylpiperidin-4-yl)-1-(2-(5-(4-fluorobenzyl)pyridin-2-yl)morpholino)ethan-1-one;
1-(2-(5-(4-fluorobenzyl)pyrazin-2-yl)morpholino)-3-(1H-indazol-1-yl)propan-1-one;
1-(2-(5-(4-fluorobenzyl)pyrimidin-2-yl)morpholino)-3-(1H-indazol-1-yl)propan-1-one;
3-(1H-indazol-1-yl)-1-(2-(5-(3-methoxyphenyl)pyridin-2-yl)morpholino)propan-1-one;
1-(2-(5-(4-fluorophenoxy)pyridin-2-yl)morpholino)-3-(1H-indazol-1-yl)propan-1-one;
2-(1-ethylpiperidin-4-yl)-1-(2-(6-(3-methoxyphenyl)pyridin-3-yl)morpholino)ethan-1-one;
2-(3-ethyl-3-azabicyclo[3.1.0]hexan-6-yl)-1-(2-(5-(3-methoxyphenyl)pyridin-2-yl)morpholino)ethan-1-one;
1-(2-(4-(4-fluorobenzyl)phenyl)morpholino)-3-(1H-pyrazol-1-yl)propan-1-one;
4-(3-(1H-indazol-1-yl)propyl)-2-(4-(4-fluorobenzyl)phenyl)morpholine;
2-(1-ethylpiperidin-4-yl)-1-(2-(5-phenylpyridin-2-yl)morpholino)ethan-1-one; and
2-(4-ethylpiperazin-1-yl)-1-(2-(5-(3-methoxyphenyl)pyridin-2-yl)morpholino)ethan-1-one;
   or a salt, solvate, prodrug, stereoisomer, isotopologue, or tautomer thereof, or any mixtures thereof.

In certain embodiments, the subject is administered a compound selected from the group consisting of.
2-(4-(2-(3,4-dimethylphenyl)acetyl)morpholin-2-yl)-N-(2-hydroxyethyl)quinoline-4-carboxamide;
N-(2-hydroxyethyl)-2-(4-((1-methyl-1H-indol-3-yl)methyl)morpholin-2-yl)quinoline-4-carboxamide;
N-(2-methoxyethyl)-2-(4-phenethylmorpholin-2-yl)quinoline-4-carboxamide; 2-(4-((1H-indol-5-yl)methyl)morpholin-2-yl)-N-(2-hydroxyethyl)-N-methylquinoline-4-carboxamide;
2-(4-benzoylmorpholin-2-yl)-N-isobutylquinoline-4-carboxamide;
N-isopropyl-2-(4-(quinolin-4-ylmethyl)morpholin-2-yl)quinoline-4-carboxamide;
N-methyl-2-(4-((3-phenyl-1H-pyrazol-4-yl)methyl)morpholin-2-yl)quinoline-4-carboxamide;
1-(1′-(2-(2-fluorophenyl)acetyl)-[2,4′-bipiperidin]-1-yl)-3-(1H-indazol-1-yl)propan-1-one;
2-(4-(2-(4-fluorophenyl)acetyl)morpholin-2-yl)-N-isobutylquinoline-4-carboxamide;
2-(4-((1H-indol-5-yl)methyl)morpholin-2-yl)-N-(2-methoxyethyl)quinoline-4-carboxamide;
N-(2-hydroxyethyl)-2-(4-(quinolin-6-ylmethyl)morpholin-2-yl)quinoline-4-carboxamide
N-isobutyl-2-(4-(2-phenylacetyl)morpholin-2-yl)quinoline-4-carboxamide;
3-(6-(4-(4,6-dimethylpyrimidin-2-yl)morpholin-2-yl)pyridin-3-yl)propenamide;
N,N-dimethyl-2-(2-(pyridin-2-yl)morpholino)acetamide;
3-(1H-indazol-1-yl)-1-(2-(5-(4-methoxybenzyl)pyridin-2-yl)pyrrolidin-1-yl)propan-1-one;
1-(2-(6-(3-fluorobenzyl)pyrazin-2-yl)pyrrolidin-1-yl)-3-(1H-indazol-1-yl)propan-1-one;
4-(3-(1H-indazol-1-yl)propanoyl)-3-(3-(pyridin-4-yl)benzyl)piperazin-2-one;
(2-(6-(dimethylamino)pyridin-2-yl)morpholino)(5-methylpyridin-3-yl)methanone;
1-(2-(pyrazin-2-yl)morpholino)-2-(pyridin-3-yl)ethan-1-one; and
N-(2-hydroxyethyl)-N-methyl-2-(morpholin-2-yl)quinoline-4-carboxamide;
   or a salt, solvate, prodrug, stereoisomer, isotopologue, or tautomer thereof, or any mixtures thereof

### Compounds

In one aspect, the present disclosure provides a compound of formula (II), or a salt, solvate, prodrug, stereoisomer, isotopologue, or tautomer thereof or any mixtures thereof:

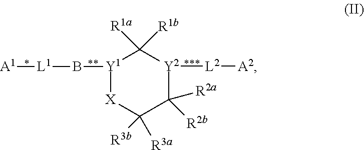

wherein:
A¹ is selected from the group consisting of optionally substituted phenyl and optionally substituted C₁-C₁₀ heterocyclyl;
A² is selected from the group consisting of optionally substituted phenyl, optionally substituted C₁-C₁₀ heterocyclyl, and NH (optionally substituted C₂-C₈ heterocyclyl);
B is

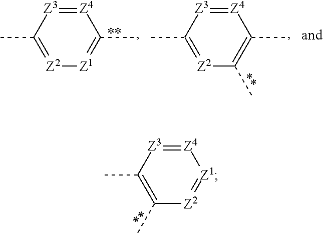

L¹ is selected from the group consisting of a bond, —NR⁴—, —O—, and optionally substituted C₁-C₃ alkylenyl;
L² is selected from the group consisting of a bond, —C(═O)—, —C(═O)O—, —C(═O)(optionally substituted C₁-C₃ alkylenyl)-, and optionally substituted C₁-C₃ alkylenyl;
X is selected from the group consisting of —O— and —C(R^{5a})(R^{5b})—,
Y¹ and Y² are each independently selected from the group consisting of N and CR⁶,
   wherein Y¹ and Y² cannot be both N, and
   wherein if X is 0, then Y¹ is CR⁶;
Z¹ is selected from the group consisting of CR⁷a and N, Z² is selected from the group consisting of CR⁷b and N, Z³ is selected from the group consisting of CR^{7c} and N, and Z⁴ is selected from the group consisting of CR^{7d} and N,
   wherein 0-2 selected from the group consisting of Z¹, Z², Z³, and Z⁴ are N;
R^{1a}, R^{1b}, R^{2a}, R^{2b}, R^{3a}, and R^{3b} are each independently selected from the group consisting of H, optionally substituted C₁-C₆ alkyl, optionally substituted C₃-C₈ cycloalkyl, optionally substituted C₁-C₆ alkoxy, and halogen;
R⁴ is selected from the group consisting of H and optionally substituted C₁-C₆ alkyl;
R^{5a} and R^{5b} are each independently selected from the group consisting of H, optionally substituted C₁-C₆ alkyl, optionally substituted C₃-C₈ cycloalkyl, optionally substituted C₁-C₆ alkoxy, and halogen;
R⁶ is selected from the group consisting of H, optionally substituted C₁-C₆ alkyl, optionally substituted C₃-C₈ cycloalkyl, optionally substituted C₁-C₆ alkoxy, and halogen;
R^{7a}, R^{7b}, R^{7c}, and R^{7d} are each independently selected from the group consisting of H, optionally substituted C₁-C₆ alkyl, optionally substituted C₃-C₈ cycloalkyl, optionally substituted C₁-C₆ alkoxy, optionally substituted phenyl, optionally substituted C₂-C₈ heterocyclyl, halogen, CN, NO₂, OR^{a}, N(R^{a})(R^{b}), C(═O)R^{a}, C(═O)OR^{a}, OC(═O)R^{a}, C(═O)N(R^{a})(R^{b}), S(═O)₂N(R^{a})(R^{b}), NR^{a}C(═O)R^{b}, and NR^{a}S(═O)₂R^{b},
   wherein two vicinal substituents selected from the group consisting of R^{7a}, R^{1b}, R^{7c}, and R^{7d} can combine with the atoms to which they are bound to form an optionally substituted phenyl or optionally substituted C₂-C₈ heterocyclyl;
R^{a} and R^{b} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl;
* indicates a bond between A¹ and L¹;
** indicates a bond between B and Y¹; and
*** indicates a bond between Y² and L².

In certain embodiments, the compound of formula (II) is selected from the group consisting of:

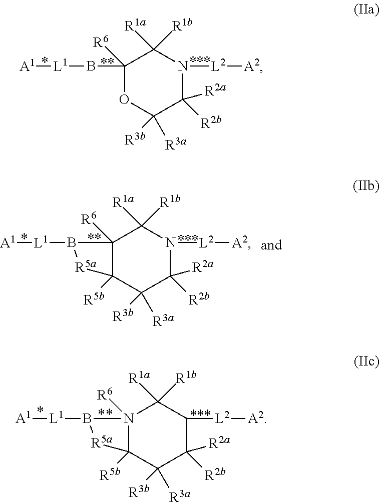

In certain embodiments, the compound of formula (II) is selected from the group consisting of:

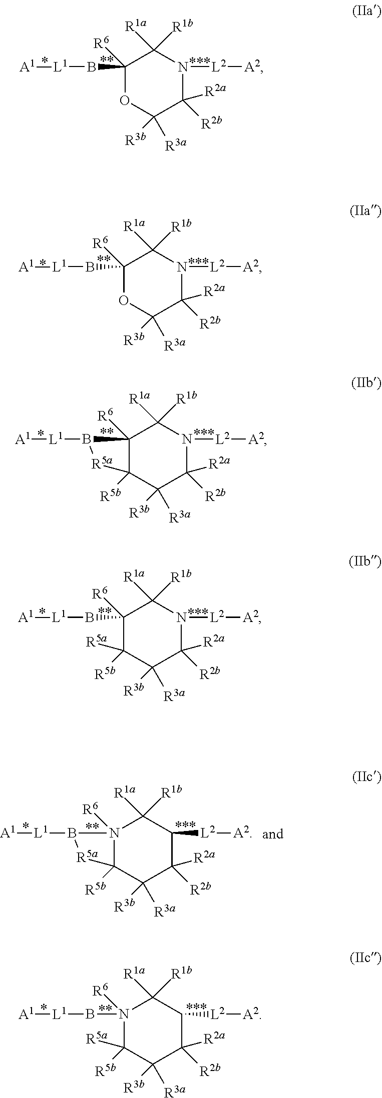

In certain embodiments, R^{1a}, R^{1b}, R^{2a}, R^{2b}, R^{3a}, and R^{3b} are each independently H. In certain embodiments, R^{1a}, R^{1b}, R^{2a}, R^{2b}, R^{3a}, R^{3b}, R^{5a}, and R^{5b} are each independently H. In certain embodiments, R^{1a}, R^{1b}, R^{2a}, R^{2b}, R^{3a}, R^{3b}, and R⁶ are each independently H. In certain embodiments, R^{1a}, R^{1b}, R^{2a}, R^{2b}, R^{3a}, R^{3b}, R^{5a}, R^{5b}, and R⁶ are each independently H.

In certain embodiments, B is

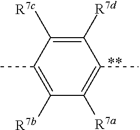

In certain embodiments, B is

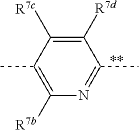

In certain embodiments, B is

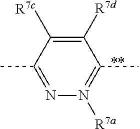

In certain embodiments, B is

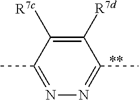

In certain embodiments, B is

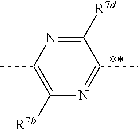

In certain embodiments, B is

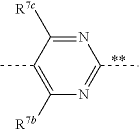

In certain embodiments, B is

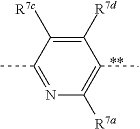

In certain embodiments, B is

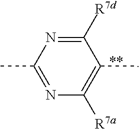

In certain embodiments, B is

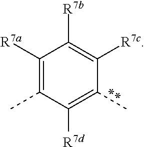

In certain embodiments, B is

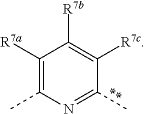

In certain embodiments, B is

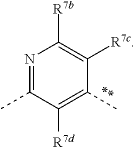

In certain embodiments, B is

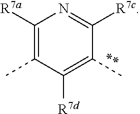

In certain embodiments, B is

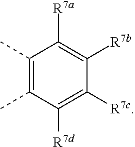

In certain embodiments, B is

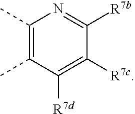

In certain embodiments, B is

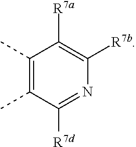

In certain embodiments, at least one selected from the group consisting of R^{7a}, R^{7b}, R^{7c}, and R^{7d} is H. In certain embodiments, at least two selected from the group consisting of R^{7a}, R^{7b}, R^{7c}, and R^{7d} are H. In certain embodiments, at least three selected from the group consisting of R^{7a}, R^{7b}, R^{7c}, and R^{7d} are H. In certain embodiments, each of R^{7a}, R^{1b}, R^{7c}, and R^{7d} are H. In certain embodiments, two vicinal substituents selected from the group consisting of R^{7a}, R^{7b}, R^{7c}, and R^{7d} combine to form phenyl.

In certain embodiments, B is

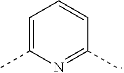

In certain embodiments, B is

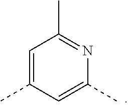

In certain embodiments, B is

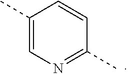

In certain embodiments, B is

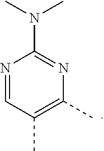

In certain embodiments, B is

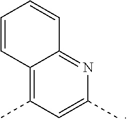

In certain embodiments, the phenyl in A¹ is optionally substituted with at least one substituent selected from the group consisting of methyl, trifluoromethyl, fluorine, chlorine, methoxy, and C(═O)NH₂. In certain embodiments, the heterocyclyl in A¹ is optionally substituted with at least one substituent selected from the group consisting of methyl, trifluoromethyl, fluorine, chlorine, methoxy, and C(═O)NH₂.

In certain embodiments, A¹ is

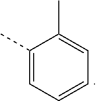

In certain embodiments, A¹ is

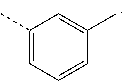

In certain embodiments, A¹ is

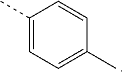

In certain embodiments, A¹ is

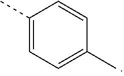

In certain embodiments, A¹ is

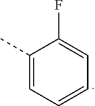

In certain embodiments, A¹ is

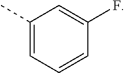

In certain embodiments, A¹ is

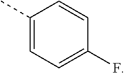

In certain embodiments, A¹ is

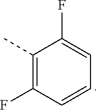

In certain embodiments, A¹ is

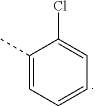

In certain embodiments, A¹ is

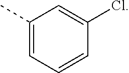

In certain embodiments, A¹ is

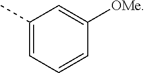

In certain embodiments, A¹ is

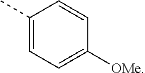

In certain embodiments, A¹ is

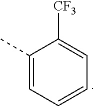

In certain embodiments, A¹ is

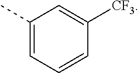

In certain embodiments, A¹ is

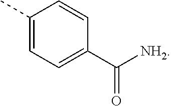

In certain embodiments, A¹ is

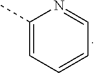

In certain embodiments, A¹ is

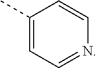

In certain embodiments, A¹ is

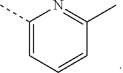

In certain embodiments, A¹ is

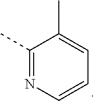

In certain embodiments, A¹ is

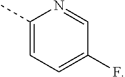

In certain embodiments, A¹ is

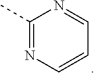

In certain embodiments, A¹ is

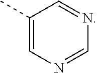

In certain embodiments, A¹ is

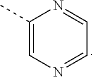

In certain embodiments, A¹ is

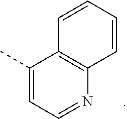

In certain embodiments, A¹ is

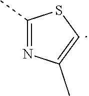

In certain embodiments, A¹ is

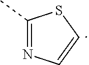

In certain embodiments, A¹ is

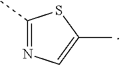

In certain embodiments, A¹ is

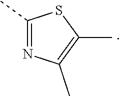

In certain embodiments, A¹ is

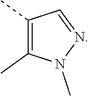

In certain embodiments, A¹ is

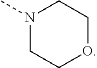

In certain embodiments, A² is selected from the group consisting of:

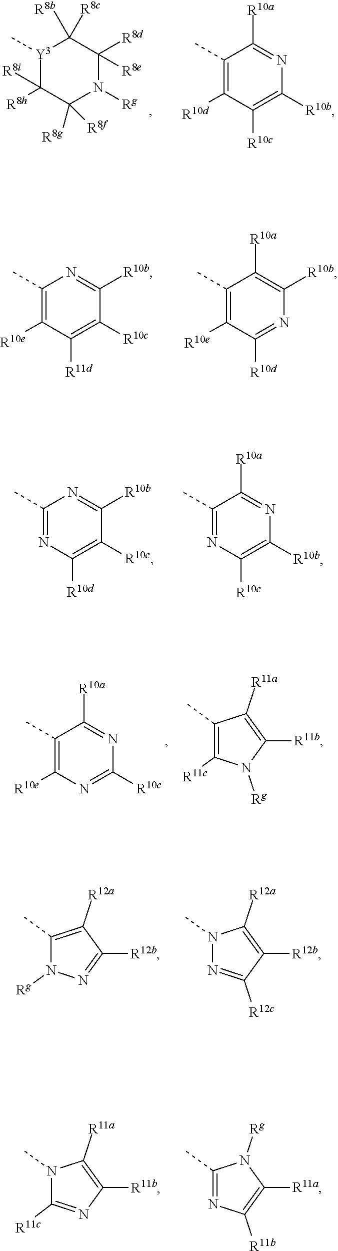

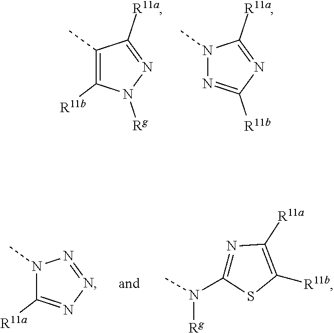

wherein:
R^{8a}, R^{8b}, R^{8c}, R^{8d}, R^{8c}, R^{8f}, R^{8g}, R^{8h}, and R⁸ⁱ are each independently selected from the group consisting of H, optionally substituted C₁-C₆ alkyl, optionally substituted C₃-C₈ cycloalkyl, and halogen,
   wherein any two substituents selected from the group consisting of R^{Ba}, R^{8b}, R^{8c}, R^{8d}, R^{8e}, R^{8f}, R^{8g}, R^{8h}, and R⁸ⁱ may combine with the atoms to which they are bound to form an optionally substituted C₃-C₈ cycloalkyl or optionally substituted C₂-C₈ heterocyclyl;
R⁹ is selected from the group consisting of H, optionally substituted C₁-C₆ alkyl, optionally substituted C₁-C₆ cycloalkyl, and optionally substituted benzyl;
R^{10a}, R^{10b}, R^{10c}, R^{10d}, and R^{10e}, if present, are each independently selected from the group consisting of H, optionally substituted C₁-C₆ alkyl, optionally substituted C₃-C₈ cycloalkyl, halogen, N(R^{a})(R^{b}), CN, and NO₂,
   wherein no more than one of R^{10a}, R^{10b}, R^{10c}, R^{10d} and R^{10e} is N(R^{a})(R^{b}), and two vicinal substituents selected from the group consisting of R^{10a}, R^{10b}, R^{10c}, R^{10d}, and R^{10c} can combine with the atoms to which they are bound to form an optionally substituted phenyl or optionally substituted C₂-C₈ heterocyclyl;
R^{11a}, R^{11b}, and R^{11c}, if present, are each independently selected from the group consisting of H, optionally substituted C₁-C₆ alkyl, optionally substituted C₃-C₈ cycloalkyl, halogen, CN, and NO₂,
   wherein two vicinal substituents selected from the group consisting of R^{11a}, R^{11b}, and R^{11c} can combine with the atoms to which they are bound to form an optionally substituted phenyl or optionally substituted C₂-C₈ heterocyclyl; and
Y³ is selected from the group consisting of R^{8a} and N.

In certain embodiments, A² is

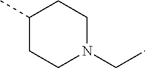

In certain embodiments, A² is

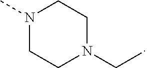

In certain embodiments, A² is

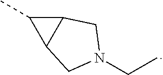

In certain embodiments, A² is

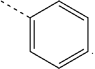

In certain embodiments, A² is

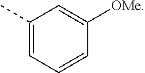

In certain embodiments, A² is

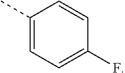

In certain embodiments, A² is

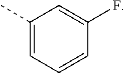

In certain embodiments, A² is

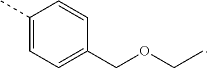

In certain embodiments, A² is

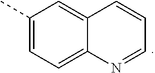

In certain embodiments, A² is

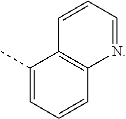

In certain embodiments, A² is

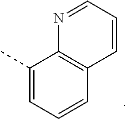

In certain embodiments, A² is

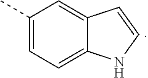

In certain embodiments, A² is

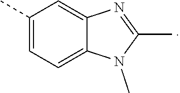

In certain embodiments, A² is

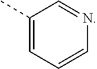

In certain embodiments, A² is

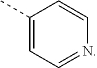

In certain embodiments, A² is

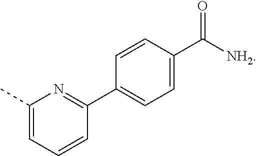

In certain embodiments, A² is

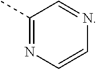

In certain embodiments, A² is

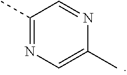

In certain embodiments, A² is

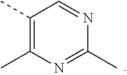

In certain embodiments, A² is

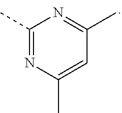

In certain embodiments, A² is

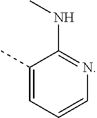

In certain embodiments, A² is

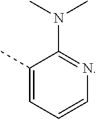

In certain embodiments, A² is

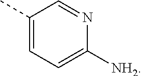

In certain embodiments, A² is

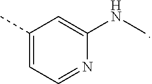

In certain embodiments, A² is

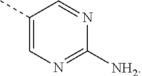

In certain embodiments, A² is

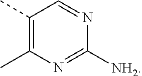

In certain embodiments, A² is

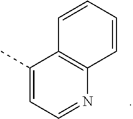

In certain embodiments, A² is

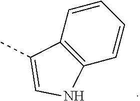

In certain embodiments, A² is

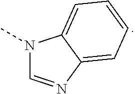

In certain embodiments, A² is

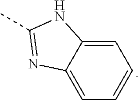

In certain embodiments, A² is

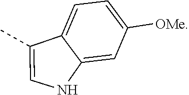

In certain embodiments, A² is is

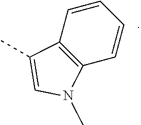

In certain embodiments, A² is

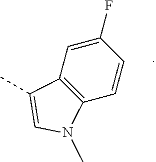

In certain embodiments, A² is

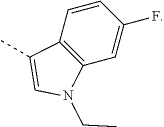

In certain embodiments, A² is

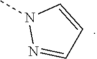

In certain embodiments, A² is

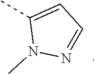

In certain embodiments, A² is

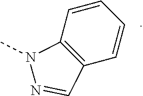

In certain embodiments, A² is

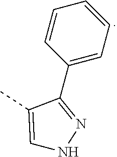

In certain embodiments, A² is

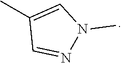

In certain embodiments, A² is

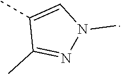

In certain embodiments, A² is

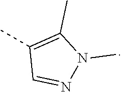

In certain embodiments, A² is

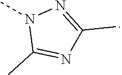

In certain embodiments, A² is

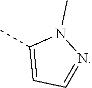

In certain embodiments, A² is

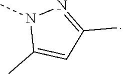

In certain embodiments, A² is

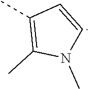

In certain embodiments, A² is

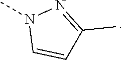

In certain embodiments, A² is

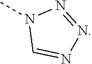

In certain embodiments, A² is

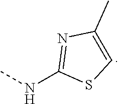

In certain embodiments, A² is

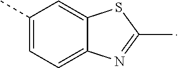

In certain embodiments, L¹ is a bond. In certain embodiments, L¹ is —NH—. In certain embodiments, L¹ is —O—. In certain embodiments, L¹ is —CH₂—.

In certain embodiments, L² is a bond. In certain embodiments, L² is —CH₂—. In certain embodiments, L² is —CH₂CH₂—. In certain embodiments, L² is —CH₂CH₂CH₂—. In certain embodiments, L² is —C(═O)—. In certain embodiments, L² is

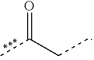

In certain embodiments, L² is

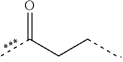

In certain embodiments, each occurrence of optionally substituted alkyl, optionally substituted cycloalkyl, optionally substituted heterocyclyl, optionally substituted alkylenyl, optionally substituted alkoxy, optionally substituted benzyl, and optionally substituted phenyl, if present, is optionally substituted with at least one substituent selected from the group consisting of C₁-C₆ alkyl, C₁-C₆ heteroalkyl, C₃-C₈ cycloalkyl, C₁-C₆ haloalkyl, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, wherein each occurrence of R′ and R″ is independently selected from the group consisting of H, C₁-C₆ alkyl, C₃-C₈ cycloalkyl, C₁-C₆ haloalkyl, benzyl, and phenyl.

In certain embodiments, the compound of formula (II) is not 1-(2-(5-(4-fluorobenzyl)pyridin-2-yl)morpholino)-3-(1H-indazol-1-yl)propan-1-one. In certain embodiments, the compound of formula (II) is not 2-(1-ethylpiperidin-4-yl)-1-(2-(5-(3-methoxyphenyl)pyridin-2-yl)morpholino)ethan-1-one. In certain embodiments, the compound of formula (II) is not (2-(5-(4-methylbenzyl)pyridin-2-yl)morpholino)(5-methylpyrazin-2-yl)methanone. In certain embodiments, the compound of formula (II) is not N-methyl-3-((2-(5-(3-(trifluoromethyl)benzyl)pyridin-2-yl)morpholino)methyl)pyridin-2-amine. In certain embodiments, the compound of formula (11) is not 4-(pyridin-3-ylmethyl)-2-(5-(3-(trifluoromethyl)benzyl)pyridin-2-yl)morpholine. In certain embodiments, the compound of formula (II) is not 2-(6-(4-fluorobenzyl)pyridin-2-yl)-4-(quinolin-6-ylmethyl)morpholine. In certain embodiments, the compound of formula (II) is not 4-(6-(4-(quinolin-4-ylmethyl)morpholin-2-yl)pyridin-2-yl)benzamide. In certain embodiments, the compound of formula (II) is not morpholino(2-(4-phenethylmorpholin-2-yl)quinolin-4-yl)methanone. In certain embodiments, the compound of formula (II) is not (2-(4-(3-methoxybenzyl)morpholin-2-yl)quinolin-4-yl)(morpholino)methanone. In certain embodiments, the compound of formula (II) is not 2-(4-(2-(3,4-dimethylphenyl)acetyl)morpholin-2-yl)-N-(2-hydroxyethyl)quinoline-4-carboxamide. In certain embodiments, the compound of formula (II) is not 4-(6-(4-(4-(4-fluorophenyl)butanoyl)morpholin-2-yl)pyridin-3-yl)benzamide. In certain embodiments, the compound of formula (II) is not 3-(1H-indol-3-yl)-1-(2-(6-(4-methoxyphenyl)pyridin-2-yl)morpholino)propan-1-one. In certain embodiments, the compound of formula (II) is not N-(2-hydroxyethyl)-2-(4-((1-methyl-1H-indol-3-yl)methyl)morpholin-2-yl)quinoline-4-carboxamide. In certain embodiments, the compound of formula (II) is not N-(2-methoxyethyl)-2-(4-phenethylmorpholin-2-yl)quinoline-4-carboxamide. In certain embodiments, the compound of formula (II) is not 3-(1H-benzo[d]imidazol-2-yl)-1-(2-(6-(2-fluorobenzyl)pyridin-2-yl)morpholino)propan-1-one. In certain embodiments, the compound of formula (II) is not 3-(1H-benzo[d]imidazol-2-yl)-1-(2-(5-(2-fluorobenzyl)pyridin-2-yl)morpholino)propan-1-one. In certain embodiments, the compound of formula (II) is not (2-(6-(2-chlorobenzyl)pyridin-2-yl)morpholino)(quinolin-5-yl)methanone. In certain embodiments, the compound of formula (II) is not (2-(6-benzylpyridin-2-yl)morpholino)(quinolin-8-yl)methanone. In certain embodiments, the compound of formula (II) is not 2-(5-(4-methoxybenzyl)pyridin-2-yl)-4-(quinolin-5-ylmethyl)morpholine. In certain embodiments, the compound of formula (II) is not 2-(4-benzylmorpholin-2-yl)-N-(thiazol-2-yl)quinoline-4-carboxamide. In certain embodiments, the compound of formula (II) is not (2-(5-(2-chlorobenzyl)pyridin-2-yl)morpholino)(quinolin-8-yl)methanone. In certain embodiments, the compound of formula (II) is not 2-(4-((1H-indol-5-yl)methyl)morpholin-2-yl)-N-(2-hydroxyethyl)-N-methylquinoline-4-carboxamide. In certain embodiments, the compound of formula (II) is not 2-(4-benzoylmorpholin-2-yl)-N-isobutylquinoline-4-carboxamide. In certain embodiments, the compound of formula (II) is not 5-(4-fluorophenyl)-N,N-dimethyl-4-(4-(quinolin-4-ylmethyl)morpholin-2-yl)pyrimidin-2-amine. In certain embodiments, the compound of formula (II) is not 1-(2-(2-(dimethylamino)-5-(3-fluorophenyl)pyrimidin-4-yl)morpholino)-2-(4-fluorophenyl)ethan-1-one. In certain embodiments, the compound of formula (11) is not N-isopropyl-2-(4-(quinolin-4-ylmethyl)morpholin-2-yl)quinoline-4-carboxamide. In certain embodiments, the compound of formula (II) is not N-methyl-2-(4-((3-phenyl-1H-pyrazol-4-yl)methyl)morpholin-2-yl)quinoline-4-carboxamide. In certain embodiments, the compound of formula (II) is not 1-(2-(6-(2,6-difluorophenyl)pyridin-2-yl)morpholino)-3-(1H-indol-3-yl)propan-1-one. In certain embodiments, the compound of formula (II) is not (2-(4-benzylmorpholin-2-yl)quinolin-4-yl)(morpholino)methanone. In certain embodiments, the compound of formula (II) is not 2-(4-(2-(4-fluorophenyl)acetyl)morpholin-2-yl)-N-isobutylquinoline-4-carboxamide. In certain embodiments, the compound of formula (II) is not 2-(4-((1H-indol-5-yl)methyl)morpholin-2-yl)-N-(2-methoxyethyl)quinoline-4-carboxamide. In certain embodiments, the compound of formula (II) is not N-(2-hydroxyethyl)-2-(4-(quinolin-6-ylmethyl)morpholin-2-yl)quinoline-4-carboxamide. In certain embodiments, the compound of formula (II) is not phenyl(2-(5-(3-(trifluoromethyl)benzyl)pyridin-2-yl)morpholino)methanone. In certain embodiments, the compound of formula (II) is not 1-(2-(5-(2-fluorobenzyl)pyridin-2-yl)morpholino)-3-(1H-indol-3-yl)propan-1-one. In certain embodiments, the compound of formula (II) is not 2-(5-(4-methoxybenzyl)pyridin-2-yl)-4-(quinolin-4-ylmethyl)morpholine. In certain embodiments, the compound of formula (II) is not 1-(2-(6-(2-fluorobenzyl)pyridin-2-yl)morpholino)-2-(3-fluorophenyl)-2-methylpropan-1-one. In certain embodiments, the compound of formula (II) is not N-isobutyl-2-(4-(2-phenylacetyl)morpholin-2-yl)quinoline-4-carboxamide. In certain embodiments, the compound of formula (II) is not 1-(2-(4-(3-fluorobenzyl)-6-methylpyridin-2-yl)morpholino)-2-(4-fluorophenyl)ethan-1-one. In certain embodiments, the compound of formula (II) is not 3-(6-(4-(4,6-dimethylpyrimidin-2-yl)morpholin-2-yl)pyridin-3-yl)propenamide. In certain embodiments, the compound of formula (II) is not 4-((1,5-dimethyl-1H-pyrazol-4-yl)methyl)-2-(5-(pyrimidin-5-yl)pyridin-2-yl)morpholine. In certain embodiments, the compound of formula (II) is not (2-(6-(2-fluorobenzyl)pyridin-2-yl)morpholino)(2-(methylamino)pyridin-4-yl)methanone. In certain embodiments, the compound of formula (II) is not N-(6-(4-(quinolin-5-ylmethyl)morpholin-2-yl)pyridin-2-yl)pyrazin-2-amine. In certain embodiments, the compound of formula (II) is not N-(6-(4-((6-methoxy-1-methyl-1H-indol-3-yl)methyl)morpholin-2-yl)pyridin-2-yl)-4-methylthiazol-2-amine. In certain embodiments, the compound of formula (II) is not N-(6-(4-((2-(dimethylamino)pyridin-3-yl)methyl)morpholin-2-yl)pyridin-2-yl)-4-methylthiazol-2-amine. In certain embodiments, the compound of formula (II) is not 4-methyl-N-(6-(4-(3-methylbenzyl)morpholin-2-yl)pyridin-2-yl)thiazol-2-amine. In certain embodiments, the compound of formula (TI) is not 2-(6-(4-methoxyphenyl)pyridin-2-yl)-4-(quinolin-5-ylmethyl)morpholine. In certain embodiments, the compound of formula (II) is not 2-(6-(2-fluorobenzyl)pyridin-2-yl)-4-(quinolin-5-ylmethyl)morpholine. In certain embodiments, the compound of formula (II) is not 2-(1H-benzo[d]imidazol-1-yl)-1-(2-(4-(2-fluorobenzyl)-6-methylpyridin-2-yl)morpholino)ethan-1-one. In certain embodiments, the compound of formula (II) is not morpholino(2-(4-(3-phenylpropyl)morpholin-2-yl)quinolin-4-yl)methanone. In certain embodiments, the compound of formula (II) is not (2-(6-(3-methoxybenzyl)pyridin-2-yl)morpholino)(quinolin-8-yl)methanone. In certain embodiments, the compound of formula (II) is not 3-(3-((2-(6-(3-fluorophenyl)pyridin-2-yl)morpholino)methyl)-1H-indol-1-yl)propan-1-ol. In certain embodiments, the compound of formula (II) is not 3-(1H-benzo[d]imidazol-1-yl)-1-(2-(6-(4-fluorobenzyl)pyridin-2-yl)morpholino)propan-1-one. In certain embodiments, the compound of formula (II) is not 2-(1H-indol-3-yl)-1-(2-(6-(3-methoxybenzyl)pyridin-2-yl)morpholino)ethan-1-one. In certain embodiments, the compound of formula (II) is not N-(6-(4-(quinolin-4-ylmethyl)morpholin-2-yl)pyridin-2-yl)pyrazin-2-amine. In certain embodiments, the compound of formula (II) is not 5-fluoro-N-(6-(4-((6-methoxy-1-methyl-1H-indol-3-yl)methyl)morpholin-2-yl)pyridin-2-yl)pyridin-2-amine. In certain embodiments, the compound of formula (II) is not N-benzyl-N-isopropyl-2-(2-(6-(pyridin-2-ylamino)pyridin-2-yl)morpholino)acetamide. In certain embodiments, the compound of formula (II) is not (2-(6-methyl-4-((6-methylpyridin-2-yl)amino)pyridin-2-yl)morpholino)(quinolin-8-yl)methanone. In certain embodiments, the compound of formula (II) is not 1-(2-(6-(2-fluorobenzyl)pyridin-2-yl)morpholino)-3-phenylpropan-1-one. In certain embodiments, the compound of formula (II) is not 1-(3-(4-(2-fluorobenzyl)-6-methylpyridin-2-yl)piperidin-1-yl)-3-(3-methyl-1H-pyrazol-1-yl)propan-1-one. In certain embodiments, the compound of formula (II) is not 2-phenyl-1-(2-(6-(2-(trifluoromethyl)benzyl)pyridin-2-yl)morpholino)ethan-1-one. In certain embodiments, the compound of formula (II) is not N-benzyl-N-isopropyl-2-(2-(6-(pyrimidin-2-ylamino)pyridin-2-yl)morpholino)acetamide. In certain embodiments, the compound of formula (II) is not 2′-(4-(2,4-dimethylpyrimidine-5-carbonyl)morpholin-2-yl)-N,6′-dimethyl-[3,4′-bipyridine]-6-carboxamide. In certain embodiments, the compound of formula (II) is not (2-(4-isonicotinoylmorpholin-2-yl)quinolin-4-yl)(morpholino)methanone. In certain embodiments, the compound of formula (II) is not pyridin-4-yl(2-(5-(3-(trifluoromethyl)benzyl)pyridin-2-yl)morpholino)methanone. In certain embodiments, the compound of formula (II) is not 2-phenyl-1-(2-(5-(3-(trifluoromethyl)benzyl)pyridin-2-yl)morpholino)ethan-1-one. In certain embodiments, the compound of formula (II) is not (4-fluorophenyl)(2-(5-(3-(trifluoromethyl)benzyl)pyridin-2-yl)morpholino)methanone. In certain embodiments, the compound of formula (II) is not 5-fluoro-N-(6-(4-((1-methyl-1H-indol-3-yl)methyl)morpholin-2-yl)pyridin-2-yl)pyridin-2-amine. In certain embodiments, the compound of formula (II) is not N-(6-(4-((1-ethyl-6-fluoro-1H-indol-3-yl)methyl)morpholin-2-yl)pyridin-2-yl)pyrazin-2-amine. In certain embodiments, the compound of formula (II) is not 3-(1H-indazol-1-yl)-1-(2-(6-((4-methylthiazol-2-yl)amino)pyridin-2-yl)morpholino)propan-1-one. In certain embodiments, the compound of formula (II) is not N-(6-(4-((5-fluoro-1-methyl-1H-indol-3-yl)methyl)morpholin-2-yl)pyridin-2-yl)-4-methylthiazol-2-amine. In certain embodiments, the compound of formula (II) is not 5-methyl-N-(2-methyl-6-(4-(quinolin-6-ylmethyl)morpholin-2-yl)pyridin-4-yl)thiazol-2-amine. In certain embodiments, the compound of formula (II) is not 4-((1,5-dimethyl-1H-pyrazol-4-yl)methyl)-2-(6-(4-fluorobenzyl)pyridin-2-yl)morpholine. In certain embodiments, the compound of formula (II) is not 1-(2-(6-benzylpyridin-2-yl)morpholino)-3-(1H-tetrazol-1-yl)propan-1-one. In certain embodiments, the compound of formula (II) is not 4-((1,3-dimethyl-1H-pyrazol-4-yl)methyl)-2-(6-(2-fluorobenzyl)pyridin-2-yl)morpholine. In certain embodiments, the compound of formula (II) is not 1-(2-(6-benzylpyridin-2-yl)morpholino)-3-(4-fluorophenyl)propan-1-one. In certain embodiments, the compound of formula (II) is not 3-(3,5-dimethyl-1H-1,2,4-triazol-1-yl)-1-(2-(4-(4-fluorobenzyl)-6-methylpyridin-2-yl)morpholino)propan-1-one. In certain embodiments, the compound of formula (II) is not 1-(2-(6-methyl-4-(3-(trifluoromethyl)benzyl)pyridin-2-yl)morpholino)-2-(1H-pyrazol-1-yl)ethan-1-one. In certain embodiments, the compound of formula (II) is not 1-(2-(6-benzylpyridin-2-yl)morpholino)-2-(4-fluorophenyl)ethan-1-one. In certain embodiments, the compound of formula (II) is not 2-(3,5-dimethyl-1H-pyrazol-1-yl)-1-(2-(6-methyl-4-(2-methylbenzyl)pyridin-2-yl)morpholino)ethan-1-one. In certain embodiments, the compound of formula (II) is not (6-aminopyridin-3-yl)(2-(4-(2-fluorobenzyl)-6-methylpyridin-2-yl)morpholino)methanone. In certain embodiments, the compound of formula (II) is not 1-(2-(6-(3-chlorobenzyl)pyridin-2-yl)morpholino)-2-(4-fluorophenyl)ethan-1-one. In certain embodiments, the compound of formula (II) is not 4-((1,5-dimethyl-TH-pyrazol-4-yl)methyl)-2-(6-(2-methylbenzyl)pyridin-2-yl)morpholine. In certain embodiments, the compound of formula (II) is not 2-(4-benzyl-6-methylpyridin-2-yl)-4-((1,3-dimethyl-TH-pyrazol-4-yl)methyl)morpholine. In certain embodiments, the compound of formula (II) is not N,N-dimethyl-5-((2-(6-methyl-4-(4-methylbenzyl)pyridin-2-yl)morpholino)methyl)pyrimidin-2-amine. In certain embodiments, the compound of formula (II) is not N,N-dimethyl-5-((2-(6-methyl-4-(4-methylbenzyl)pyridin-2-yl)morpholino)methyl)pyrimidin-2-amine. In certain embodiments, the compound of formula (II) is not (1,2-dimethyl-1H-benzo[d]imidazol-5-yl)(2-(6-methyl-4-(pyridin-2-ylamino)pyridin-2-yl)morpholino)methanone. In certain embodiments, the compound of formula (II) is not 3-(3,5-dimethyl-1H-1,2,4-triazol-1-yl)-1-(2-(6-(4-fluorobenzyl)pyridin-2-yl)morpholino)propan-1-one. In certain embodiments, the compound of formula (II) is not 2-(4-(4-fluorobenzyl)-6-methylpyridin-2-yl)-4-((1-methyl-1H-pyrazol-4-yl)methyl)morpholine. In certain embodiments, the compound of formula (II) is not 1-(2-(6-(2,6-difluorobenzyl)pyridin-2-yl)morpholino)-2-morpholinoethan-1-one. In certain embodiments, the compound of formula (II) is not (2-(6-(2,6-difluorobenzyl)pyridin-2-yl)morpholino)(2-(methylamino)pyridin-4-yl)methanone. In certain embodiments, the compound of formula (II) is not 1-(2-(5-(3-chlorobenzyl)pyridin-2-yl)morpholino)-3-(3,5-dimethyl-1H-1,2,4-triazol-1-yl)propan-1-one. In certain embodiments, the compound of formula (II) is not 3-((2-(6-(4-methoxyphenyl)pyridin-2-yl)morpholino)methyl)-N,N-dimethylpyridin-2-amine. In certain embodiments, the compound of formula (II) is not 1-(2-(6-benzylpyridin-2-yl)morpholino)-3-phenylpropan-1-one. In certain embodiments, the compound of formula (II) is not (2-(dimethylamino)-4-methylpyrimidin-5-yl)(2-(6-methyl-4-(4-methylbenzyl)pyridin-2-yl)morpholino)methanone. In certain embodiments, the compound of formula (II) is not (2-(6-methyl-4-(3-(trifluoromethyl)benzyl)pyridin-2-yl)morpholino)(pyridin-3-yl)methanone. In certain embodiments, the compound of formula (II) is not (4-(ethoxymethyl)phenyl)(2-(6-methyl-4-((3-methylpyridin-2-yl)amino)pyridin-2-yl)morpholino)methanone. In certain embodiments, the compound of formula (II) is not 5-((2-(6-(2-chlorobenzyl)pyridin-2-yl)morpholino)methyl)-N,N-dimethylpyrimidin-2-amine. In certain embodiments, the compound of formula (II) is not 2-(1-ethylpiperidin-4-yl)-1-(2-(3′-methoxy-[1,1′-biphenyl]-4-yl)morpholino)ethan-1-one. In certain embodiments, the compound of formula (II) is not 2-(1-ethylpiperidin-4-yl)-1-(3-(5-(3-methoxyphenyl)pyridin-2-yl)piperidin-1-yl)ethan-1-one. In certain embodiments, the compound of formula (II) is not 2-(1-ethylpiperidin-4-yl)-1-(1-(5-(3-methoxyphenyl)pyridin-2-yl)piperidin-3-yl)ethan-1-one. In certain embodiments, the compound of formula (II) is not 1-ethylpiperidin-4-yl 2-(5-(3-methoxyphenyl)pyridin-2-yl)morpholine-4-carboxylate. In certain embodiments, the compound of formula (II) is not 4-(2-(1-ethylpiperidin-4-yl)ethyl)-2-(5-(3-methoxyphenyl)pyridin-2-yl)morpholine. In certain embodiments, the compound of formula (II) is not 2-(1-ethylpiperidin-4-yl)-1-(2-(5-(4-fluorobenzyl)pyridin-2-yl)morpholino)ethan-1-one. In certain embodiments, the compound of formula (II) is not 1-(2-(5-(4-fluorobenzyl)pyrazin-2-yl)morpholino)-3-(1H-indazol-1-yl)propan-1-one. In certain embodiments, the compound of formula (II) is not 1-(2-(5-(4-fluorobenzyl)pyrimidin-2-yl)morpholino)-3-(1H-indazol-1-yl)propan-1-one. In certain embodiments, the compound of formula (II) is not 3-(1H-indazol-1-yl)-1-(2-(5-(3-methoxyphenyl)pyridin-2-yl)morpholino)propan-1-one. In certain embodiments, the compound of formula (II) is not 1-(2-(5-(4-fluorophenoxy)pyridin-2-yl)morpholino)-3-(1H-indazol-1-yl)propan-1-one. In certain embodiments, the compound of formula (II) is not 2-(1-ethylpiperidin-4-yl)-1-(2-(6-(3-methoxyphenyl)pyridin-3-yl)morpholino)ethan-1-one. In certain embodiments, the compound of formula (II) is not 2-(3-ethyl-3-azabicyclo[3.1.0]hexan-6-yl)-1-(2-(5-(3-methoxyphenyl)pyridin-2-yl)morpholino)ethan-1-one. In certain embodiments, the compound of formula (II) is not 1-(2-(4-(4-fluorobenzyl)phenyl)morpholino)-3-(1H-pyrazol-1-yl)propan-1-one. In certain embodiments, the compound of formula (II) is not 4-(3-(1H-indazol-1-yl)propyl)-2-(4-(4-fluorobenzyl)phenyl)morpholine. In certain embodiments, the compound of formula (II) is not 2-(1-ethylpiperidin-4-yl)-1-(2-(5-phenylpyridin-2-yl)morpholino)ethan-1-one. In certain embodiments, the compound of formula (II) is not 2-(4-ethylpiperazin-1-yl)-1-(2-(5-(3-methoxyphenyl)pyridin-2-yl)morpholino)ethan-1-one. In certain embodiments, the compound of formula (II) is not 2-(5-(4-fluorobenzyl)pyridin-2-yl)-4-((1-methyl-1H-pyrazol-5-yl)methyl)morpholine. In certain embodiments, the compound of formula (II) is not (2-(6-(2-fluorobenzyl)pyridin-2-yl)morpholino)(pyrazin-2-yl)methanone. In certain embodiments, the compound of formula (II) is not 3-(1H-indol-3-yl)-1-(2-(5-(4-methylbenzyl)pyridin-2-yl)morpholino)propan-1-one. In certain embodiments, the compound of formula (II) is not (6-aminopyridin-3-yl)(2-(5-(4-methylbenzyl)pyridin-2-yl)morpholino)methanone. In certain embodiments, the compound of formula (II) is not (2-methylbenzo[d]thiazol-6-yl)(2-(5-(4-methylbenzyl)pyridin-2-yl)morpholino)methanone. In certain embodiments, the compound of formula (II) is not 3-((2-(6-benzylpyridin-2-yl)morpholino)methyl)-N,N-dimethylpyridin-2-amine. In certain embodiments, the compound of formula (II) is not N-(6-(4-((2-(dimethylamino)pyridin-3-yl)methyl)morpholin-2-yl)pyridin-2-yl)-4,5-dimethylthiazol-2-amine.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S.

In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C F, ¹⁵O, and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000.2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from allyl (CH₂CH═CH₂), benzyl (i.e., Bn and/or CH₂Ph), Cbz (C(═O)OCH₂Ph), Alloc (C(═O)OCH₂CH═CH₂), methyl, ethyl, t-butyl, TBDMS (Si(CH₃)₂(C(CH₃)₃)), Teoc (C(═O)OCH₂CH₂Si(CH₃)₃), Boc (C(═O)OC(CH₃)₃), PMB (4-methoxybenzyl), trityl (triphenylmethyl), acetyl, and FMOC (fluorenylmethoxycarbonyl).

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Compositions

In one aspect, the present disclosure provides a pharmaceutical composition comprising the compound of the present disclosure (i.e., the compound of formula (I) and/or (II)) and a pharmaceutically acceptable carrier.

The compositions containing the compound(s) described herein include a pharmaceutical composition comprising at least one compound as described herein and at least one pharmaceutically acceptable carrier. In some embodiments, the pharmaceutical composition comprises Kolliphor EL, and aqueous buffer, or a combination thereof. In certain embodiments, the aqueous buffer comprises phosphate buffered saline (PBS). In some embodiments, the aqueous buffer comprises 1×PBS. In certain embodiments, the pharmaceutical composition comprises about 5%, about 10%, about 15%, about 20%, about 25%, about 30%, about 35%, or about 40% Kolliphor EL. In some embodiments, the pharmaceutical composition comprises about 20% Kolliphor EL in 1×PBS.

In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration. In some embodiments, the composition is formulated as a pill, tablet, gelcap, or capsule for oral administration.

### Administration/Dosage/Formulations

The regimen of administration may affect what constitutes an effective amount. The therapeutic formulations may be administered to the subject either prior to or after the onset of the disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat the disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat the disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In some embodiments, the compound(s) described herein are administered to a patient, alone or in combination with another pharmaceutical agent, using a sustained release formulation. In some embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of the disease or disorder in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents are considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXAMPLES

Various embodiments of the present application can be better understood by reference to the following Examples which are offered by way of illustration. The scope of the present application is not limited to the Examples given herein.

### Example 1: Compound 1 is a Selective Inhibitor of Kinases Implicated in Gα₁₂ Signaling Pathways

Using the Eurofins™ platform, a kinase activity assay was performed to identify human kinases inhibited by compound 1. To determine the effects of the compound on kinase activity, varying concentrations of compound 1 (1, 10 and 100 μM) were used with one technical replicate in a standard in vitro kinase activation assay.

This platform was used to determine the effects of compound 1 on the activity of 113 unique human kinases. Thresholds for kinase activity of interest was set at less than or equal to 60% inhibition (i.e., for antagonists) or enhanced activity at or above 140% (i.e., for agonists). Further, the validity of the results were confirmed by the occurrence of a dose-dependent response.

Results indicated that 7 kinases exhibited dose dependent inhibition with administration of compound 1, with agonism observed in only 4 kinases, none of which are relevant in human airway smooth muscle. MAPKAP-K2 and PRAK/MAPKAPK5 were among the kinases inhibited in a dose-dependent manner with administration of compound 1 (Table 1). Further, MAPKAP-K2 and PRAK/MAPKAPK5 are expressed in human airway smooth muscle and are generally considered meaningful in Gα₁₂ Signaling pathways. Additionally, the data demonstrated the specificity of compound 1, as little to no effect was observed for the overwhelming majority of the 113 human kinases evaluated.

TABLE 1
Exemplary human kinase inhibition data for compound
1 at 1 μM, 10 μM, and 100 μM

| Kinase | 1 μM | 10 μM | 100 μM |
|---|---|---|---|
| Fms(h) | 95 | 94 | 64 |
| IGF-1R(h) | 103 | 91 | 61 |
| MAPKAP-K2(h) | 90 | 77 | 65 |
| mTOR(h) | 103 | 104 | 70 |
| PRAK(h)/MAPKAPK5 | 115 | 92 | 40 |
| TrkA(h) | 106 | 123 | 41 |
| ATR/ATRIP(h) | 101 | 91 | 65 |

TABLE 2
Exemplary PI3Kδ inhibition data for compound
1 at 1 μM, 10 μM, and 100 μM

| Compound 1 | | Raw Counts | Aggregate Mean | Kinase Activity (%) | | |
|---|---|---|---|---|---|---|
| 1 | μM | 15209.80926 | 16175 | 95 | 91 | 6 |
| 1 | μM | 17139.70182 | | 86 | | |
| 10 | μM | 15948.27586 | 15960 | 92 | 92 | 0 |
| 10 | μM | 15971.06288 | | 91 | | |
| 100 | μM | 17495.26814 | 17900 | 85 | 83 | 2 |
| 100 | μM | 18305.46265 | | 81 | | |

Thus, without wishing to be bound by theory, the results described herein demonstrate the potential of compound 1, or analogues thereof, as a selective bronchodilator, and indicate a mechanism of action involving calcium sensitization pathways.

### Example 2: Identification of Gα₂ Inhibitors Using a Gα₁₂ Luciferase Reporter Assay

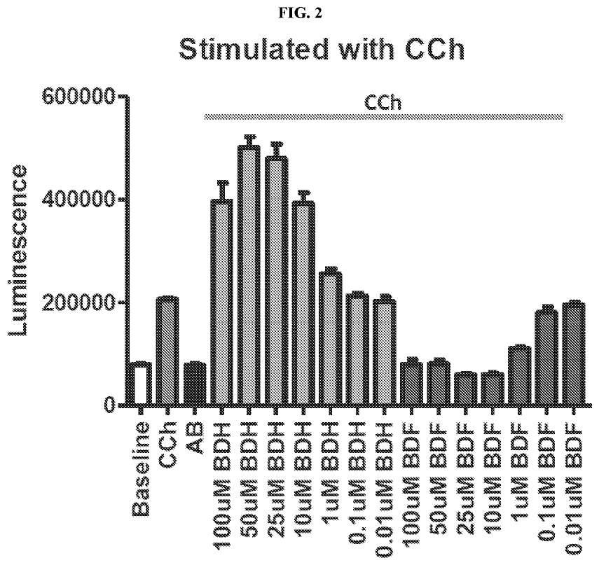

A Gα₁₂ luciferase reporter assay was used to evaluate the potential of analogues of compound 1 as bronchodilators (FIGS. 2A-2C), wherein the assay utilized hTERT-SRE telomerase reverse transcriptase immortalized airway smooth muscle cells.

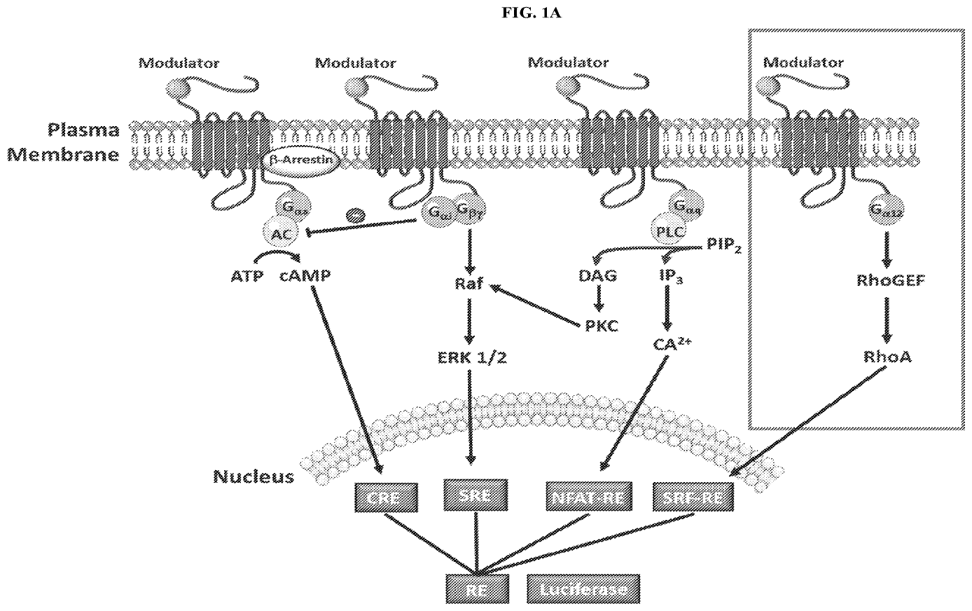

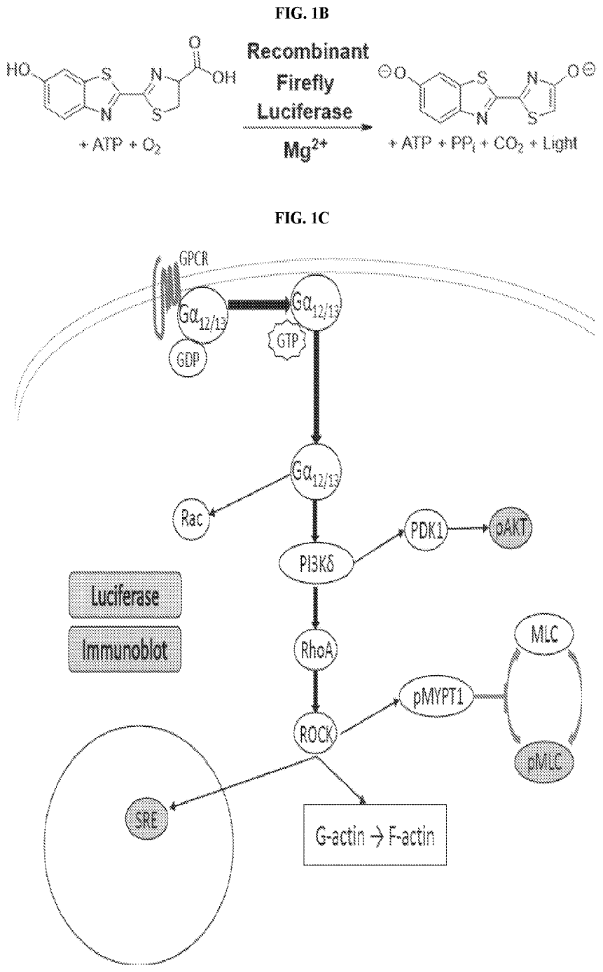

In certain embodiments, the Gα₁₂ pathway is activated with carbachol (CCh), resulting in downstream transcription of a recombinant response element (SE)/luciferase fusion protein (FIG. 1A), and consequently, a fluorescence signal (FIG. 1B). In certain embodiments, detection of a decrease in observed fluorescent signal indicates inhibition of Gα₁₂. Further, activation of the Gα₁₂ pathway results in elevated levels of phosphorylated AKT (pAKT) and MLC (pMLC) (FIG. 1C). In certain embodiments, detection of a decrease in pAKT and/or pMLC indicates inhibition of Gα₁₂.

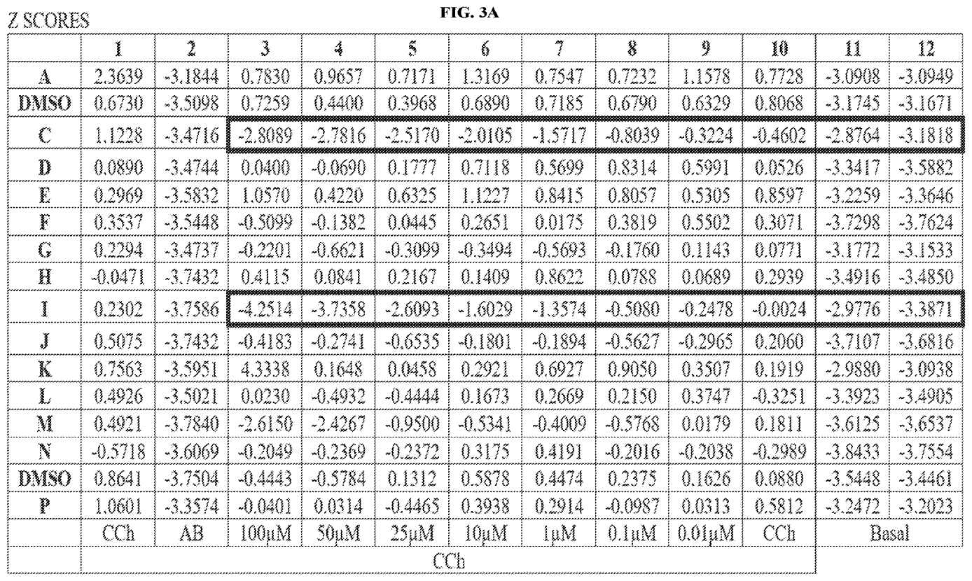

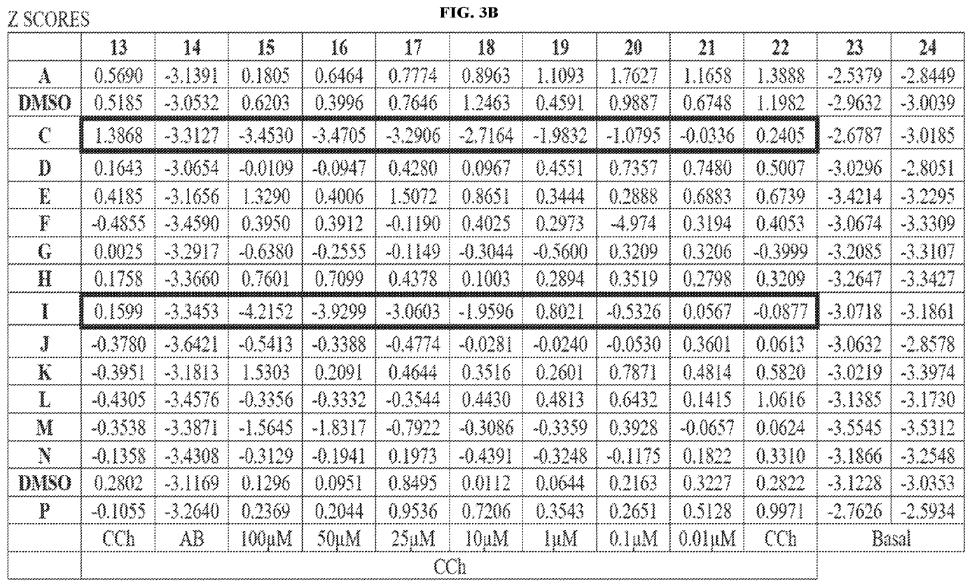

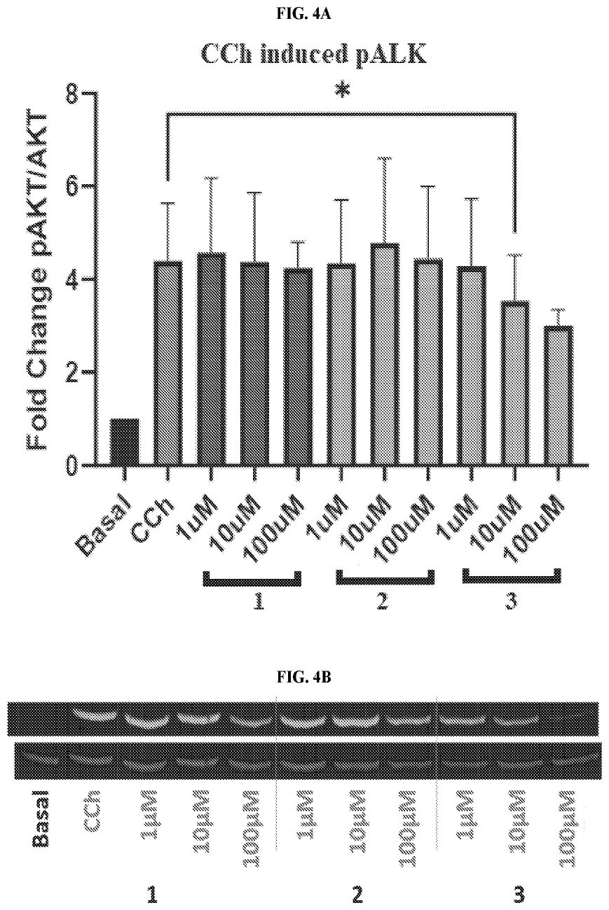

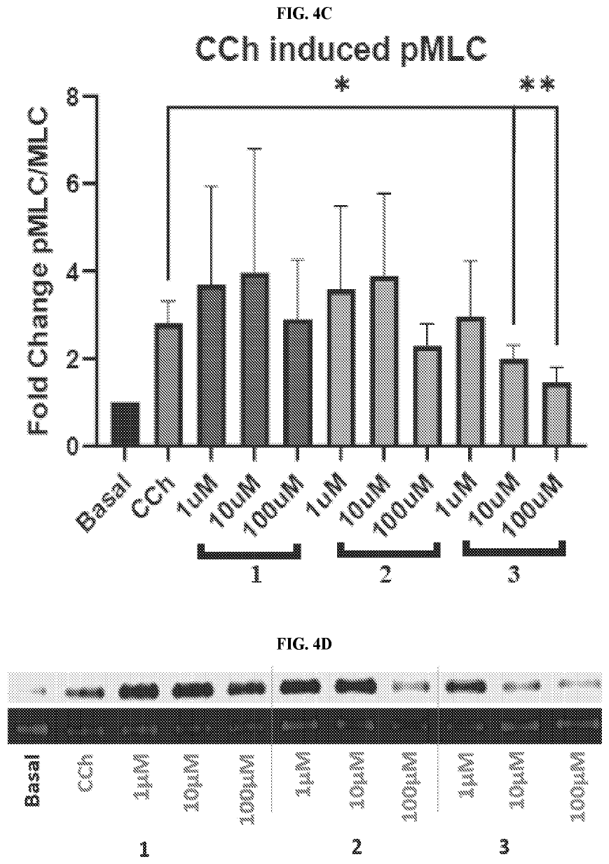

The assay was validated with compound BDH (i.e., Gα₁₂ agonist) and compound 1 (i.e., Gα₁₂ antagonist), in which dose-dependent activation and inhibition, respectively, were observed (FIG. 2). Using the assay described herein, compounds 2 and 3, inter alia, were identified as analogues of compound 1 which act as Gα₁₂ antagonists (FIGS. 3A-3B). Compounds evaluated as described herein are provided in Table 3.

In certain embodiments, compound 1 inhibits Gα₁₂ with an IC₅₀ of about 1.0 μM to about 10 μM. In embodiments, compound 2 inhibits Gα₁₂ with an IC₅₀ of about 1.0 μM to about 10 μM. In certain embodiments, compound 3 inhibits Gα₁₂ with an IC₅₀ of about 0.1 μM to about 1.0 μM. In certain embodiments, compound 4 inhibits Gα₁₂ with an IC₅₀ of about 25 μM to about 50 μM. In certain embodiments, compound 5 inhibits Gα₁₂ with an IC₅₀ of about 25 μM to about 50 μM.

TABLE 3

| Cmpd | Structure | Nomenclature |
|---|---|---|
| 1 | 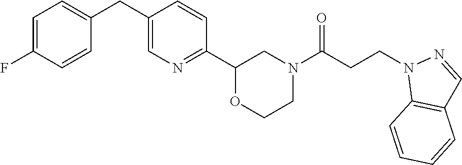 [SMILES: O=C(CCn1ncc2ccccc21)N1CCOC(c2ccc(Cc3ccc(F)cc3)cn2)C1] | 1-(2-(5-(4-fluorobenzyl)pyridin- 2-yl)morpholino)-3-(1H-indazol- 1-yl)propan-1-one |
| 2 | 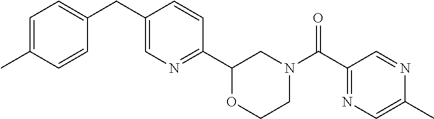 | (2-(5-(4-methylbenzyl)pyridin-2- yl)morpholino)(5-methylpyrazin- 2-yl)methanone |
| 3 | 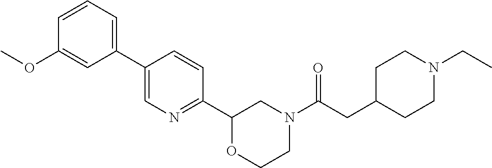 [SMILES: CCN1CCC(CC(=O)N2CCOC(c3ccc(-c4cccc(OC)c4)cn3)C2)CC1] | 2-(1-ethylpiperidin-4-yl)-1-(2-(5- (3-methoxyphenyl)pyridin-2- yl)morpholino)ethan-1-one |
| 4 | 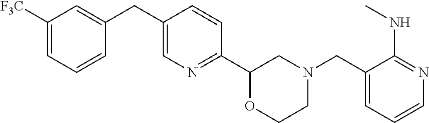 [SMILES: CNc1ncccc1CN1CCOC(c2ccc(Cc3cccc(C(F)(F)F)c3)cn2)C1] | N-methyl-3-((2-(5-(3- (trifluoromethyl)benzyl)pyridin- 2-yl)morpholino)methyl)pyridin- 2-amine |
| 5 | 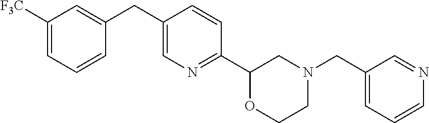 [SMILES: FC(F)(F)c1cccc(Cc2ccc(C3CN(Cc4cccnc4)CCO3)nc2)c1] | 4-(pyridin-3-ylmethyl)-2-(5-(3- (trifluoromethyl)benzyl)pyridin- 2-yl)morpholine |
| 6 | 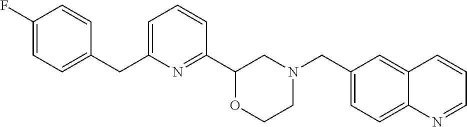 [SMILES: Fc1ccc(Cc2cccc(C3CN(Cc4ccc5ncccc5c4)CCO3)n2)cc1] | 2-(6-(4-fluorobenzyl)pyridin-2- yl)-4-(quinolin-6- ylmethyl)morpholine |
| 7 | 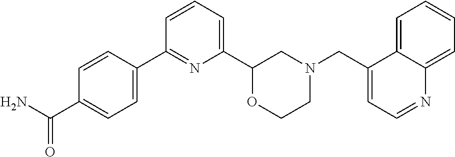 [SMILES: NC(=O)c1ccc(-c2cccc(C3CN(Cc4ccnc5ccccc45)CCO3)n2)cc1] | 4-(6-(4-(quinolin-4- ylmethyl)morpholin-2-yl)pyridin- 2-yl)benzamide |
| 8 | 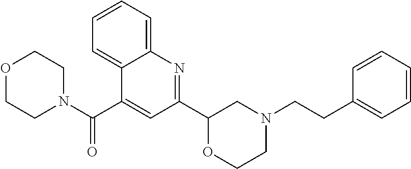 [SMILES: O=C(c1cc(C2CN(CCc3ccccc3)CCO2)nc2ccccc12)N1CCOCC1] | morpholino(2-(4- phenethylmorpholin-2- yl)quinolin-4-yl)methanone |
| 9 | 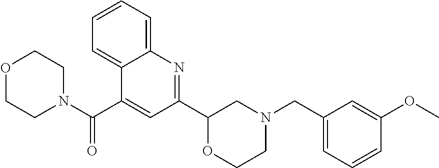 [SMILES: COc1cccc(CN2CCOC(c3cc(C(=O)N4CCOCC4)c4ccccc4n3)C2)c1] | (2-(4-(3- methoxybenzyl)morpholin-2- yl)quinolin-4- yl)(morpholino)methanone |
| 10 | 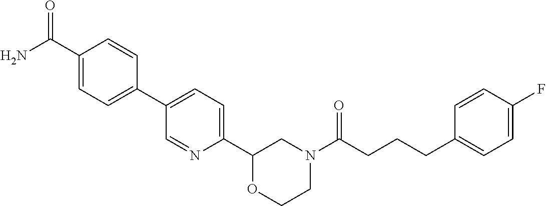 [SMILES: NC(=O)c1ccc(-c2ccc(C3CN(C(=O)CCCc4ccc(F)cc4)CCO3)nc2)cc1] | 4-(6-(4-(4-(4- fluorophenyl)butanoyl)morpholin- 2-yl)pyridin-3-yl)benzamide |
| 11 | 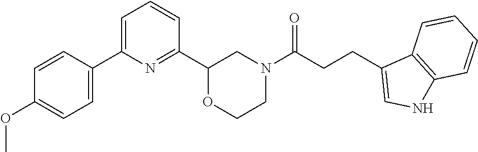 [SMILES: COc1ccc(-c2cccc(C3CN(C(=O)CCc4c[nH]c5ccccc45)CCO3)n2)cc1] | 3-(1H-indol-3-y1)-1-(2-(6-(4- methoxyphenyl)pyridin-2- yl)morpholino)propan-1-one |
| 12 | 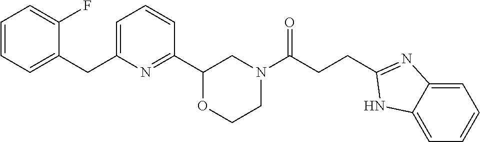 [SMILES: O=C(CCc1nc2ccccc2[nH]1)N1CCOC(c2cccc(Cc3ccccc3F)n2)C1] | 3-(1H-benzo[d]imidazol-2-yl)-1- (2-(6-(2-fluorobenzyl)pyridin-2- yl)morpholino)propan-1-one |
| 13 | 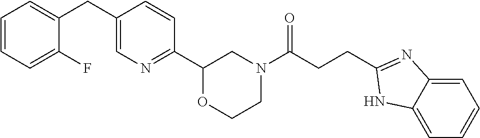 [SMILES: O=C(CCc1nc2ccccc2[nH]1)N1CCOC(c2ccc(Cc3ccccc3F)cn2)C1] | 3-(1H-benzo[d]imidazol-2-yl)-1- (2-(5-(2-fluorobenzyl)pyridin-2- yl)morpholino)propan-1-one |
| 14 | 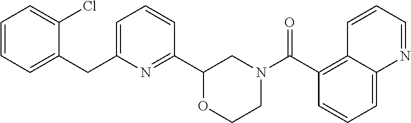 [SMILES: O=C(c1cccc2ncccc12)N1CCOC(c2cccc(Cc3ccccc3Cl)n2)C1] | (2-(6-(2-chlorobenzyl)pyridin-2- yl)morpholino)(quinolin-5- yl)methanone |
| 15 | 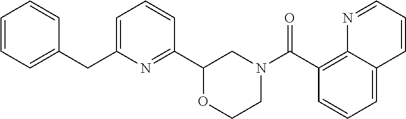 [SMILES: O=C(c1cccc2cccnc12)N1CCOC(c2cccc(Cc3ccccc3)n2)C1] | (2-(6-benzylpyridin-2- yl)morpholino)(quinolin-8- yl)methanone |
| 16 | 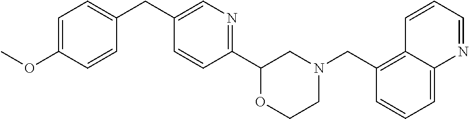 [SMILES: COc1ccc(Cc2ccc(C3CN(Cc4cccc5ncccc45)CCO3)nc2)cc1] | 2-(5-(4-methoxybenzyl)pyridin-2- yl)-4-(quinolin-5- ylmethyl)morpholine |
| 17 | 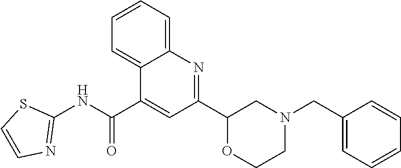 [SMILES: O=C(Nc1nccs1)c1cc(C2CN(Cc3ccccc3)CCO2)nc2ccccc12] | 2-(4-benzylmorpholin-2-yl)-N- (thiazol-2-yl)quinoline-4- carboxamide |
| 18 | 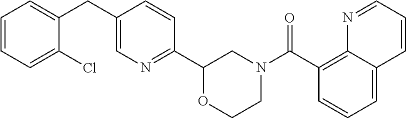 [SMILES: O=C(c1cccc2cccnc12)N1CCOC(c2ccc(Cc3ccccc3Cl)cn2)C1] | (2-(5-(2-chlorobenzyl)pyridin-2- yl)morpholino)(quinolin-8- yl)methanone |
| 19 | 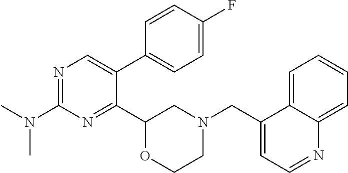 [SMILES: CN(C)c1ncc(-c2ccc(F)cc2)c(C2CN(Cc3ccnc4ccccc34)CCO2)n1] | 5-(4-fluorophenyl)-N,N- dimethyl-4-(4-(quinolin-4- ylmethyl)morpholin-2- yl)pyrimidin-2-amine |
| 20 | 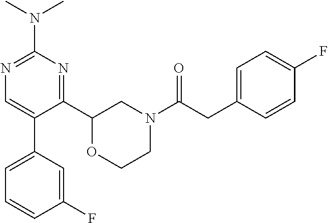 [SMILES: CN(C)c1ncc(-c2cccc(F)c2)c(C2CN(C(=O)Cc3ccc(F)cc3)CCO2)n1] | 1-(2-(2-(dimethylamino)-5-(3- fluorophenyl)pyrimidin-4- yl)morpholino)-2-(4- fluorophenyl)ethan-1-one |
| 21 | 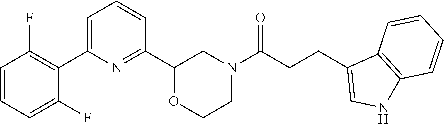 [SMILES: O=C(CCc1c[nH]c2ccccc12)N1CCOC(c2cccc(-c3c(F)cccc3F)n2)C1] | 1-(2-(6-(2,6- difluorophenyl)pyridin-2- yl)morpholino)-3-(1H-indol-3- yl)propan-1-one |
| 22 | 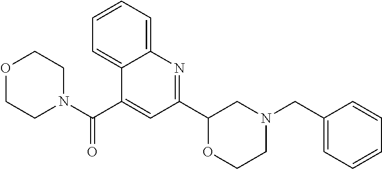 [SMILES: O=C(c1cc(C2CN(Cc3ccccc3)CCO2)nc2ccccc12)N1CCOCC1] | (2-(4-benzylmorpholin-2- yl)quinolin-4- yl)(morpholino)methanone |
| 23 | 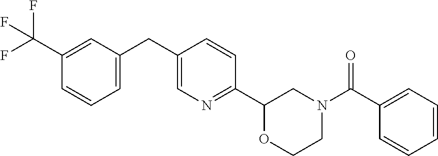 [SMILES: O=C(c1ccccc1)N1CCOC(c2ccc(Cc3cccc(C(F)(F)F)c3)cn2)C1] | phenyl(2-(5-(3- (trifluoromethyl)benzyl)pyridin- 2-yl)morpholino)methanone |
| 24 | 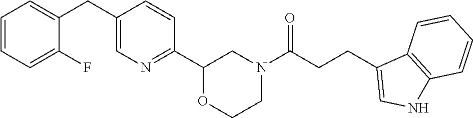 [SMILES: O=C(CCc1c[nH]c2ccccc12)N1CCOC(c2ccc(Cc3ccccc3F)cn2)C1] | 1-(2-(5-(2-fluorobenzyl)pyridin- 2-yl)morpholino)-3-(1H-indol-3- yl)propan-1-one |
| 25 | 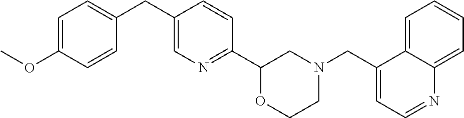 [SMILES: COc1ccc(Cc2ccc(C3CN(Cc4ccnc5ccccc45)CCO3)nc2)cc1] | 2-(5-(4-methoxybenzyl)pyridin-2- yl)-4-(quinolin-4- ylmethyl)morpholine |
| 26 | 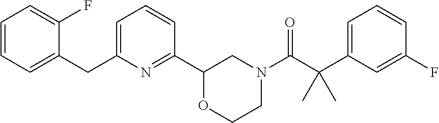 [SMILES: CC(C)(C(=O)N1CCOC(c2cccc(Cc3ccccc3F)n2)C1)c1cccc(F)c1] | 1-(2-(6-(2-fluorobenzyl)pyridin- 2-yl)morpholino)-2-(3- fluorophenyl)-2-methylpropan-1- one |
| 27 | 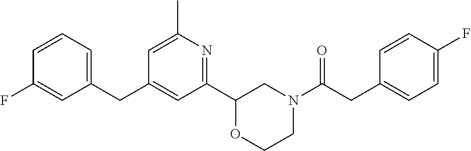 [SMILES: Cc1cc(Cc2cccc(F)c2)cc(C2CN(C(=O)Cc3ccc(F)cc3)CCO2)n1] | 1-(2-(4-(3-fluorobenzyl)-6- methylpyridin-2-yl)morpholino)- 2-(4-fluorophenyl)ethan-1-one |
| 28 | 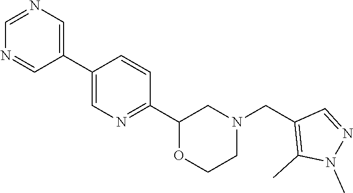 [SMILES: Cc1c(CN2CCOC(c3ccc(-c4cncnc4)cn3)C2)cnn1C] | 4-((1,5-dimethy1-1H-pyrazol-4- yl)methyl)-2-(5-(pyrimidin-5- yl)pyridin-2-yl)morpholine |
| 29 | 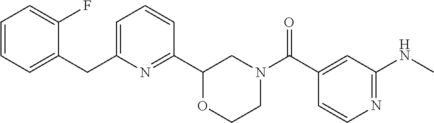 [SMILES: CNc1cc(C(=O)N2CCOC(c3cccc(Cc4ccccc4F)n3)C2)ccn1] | (2-(6-(2-fluorobenzyl)pyridin-2- yl)morpholino)(2- (methylamino)pyridin-4- yl)methanone |
| 30 | 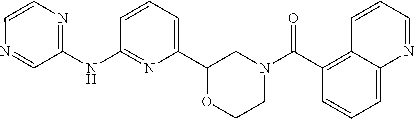 [SMILES: O=C(c1cccc2ncccc12)N1CCOC(c2cccc(Nc3cnccn3)n2)C1] | N-(6-(4-(quinolin-5- ylmethyl)morpholin-2-yl)pyridin- 2-yl)pyrazin-2-amine |
| 31 | 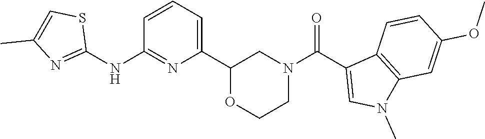 | N-(6-(4-((6-methoxy-1-methyl- 1H-indol-3-yl)methyl)morpholin- 2-yl)pyridin-2-y1)-4- methylthiazol-2-amine |
| 32 | 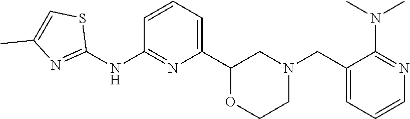 | N-(6-(4-((2- (dimethylamino)pyridin-3- yl)methyl)morpholin-2- yl)pyridin-2-yl)-4-methylthiazol- 2-amine |
| 33 | 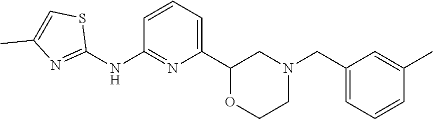 | 4-methyl-N-(6-(4-(3- methylbenzyl)morpholin-2- yl)pyridin-2-yl)thiazol-2-amine |
| 34 | 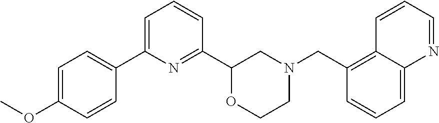 [SMILES: COc1ccc(-c2cccc(C3CN(Cc4cccc5ncccc45)CCO3)n2)cc1] | 2-(6-(4-methoxyphenyl)pyridin- 2-yl)-4-(quinolin-5- ylmethyl)morpholine |
| 35 | 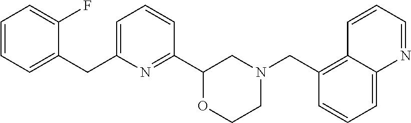 [SMILES: Fc1ccccc1Cc1cccc(C2CN(Cc3cccc4ncccc34)CCO2)n1] | 2-(6-(2-fluorobenzyl)pyridin-2- yl)-4-(quinolin-5- ylmethyl)morpholine |
| 36 | 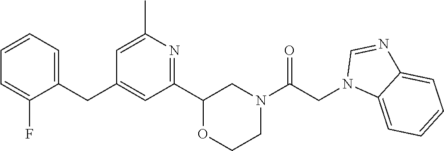 [SMILES: Cc1cc(Cc2ccccc2F)cc(C2CN(C(=O)Cn3cnc4ccccc43)CCO2)n1] | 2-(1H-benzo[d]imidazol-1-y1)-1- (2-(4-(2-fluorobenzyl)-6- methylpyridin-2- yl)morpholino)ethan-1-one |
| 37 | 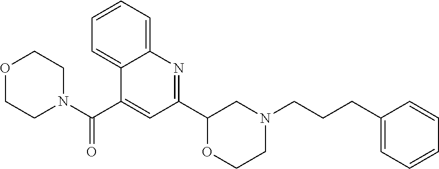 [SMILES: O=C(c1cc(C2CN(CCCc3ccccc3)CCO2)nc2ccccc12)N1CCOCC1] | morpholino(2-(4-(3- phenylpropyl)morpholin-2- yl)quinolin-4-yl)methanone |
| 38 | 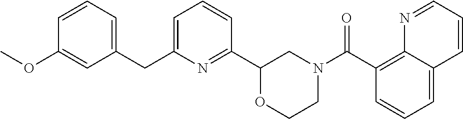 [SMILES: COc1cccc(Cc2cccc(C3CN(C(=O)c4cccc5cccnc45)CCO3)n2)c1] | (2-(6-(3-methoxybenzyl)pyridin- 2-yl)morpholino)(quinolin-8- yl)methanone |
| 39 | 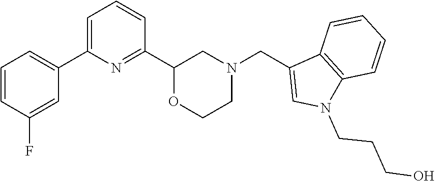 [SMILES: OCCCn1cc(CN2CCOC(c3cccc(-c4cccc(F)c4)n3)C2)c2ccccc21] | 3-(3-((2-(6-(3- fluorophenyl)pyridin-2- yl)morpholino)methyl)-1H-indol- 1-yl)propan-1-ol |
| 40 | 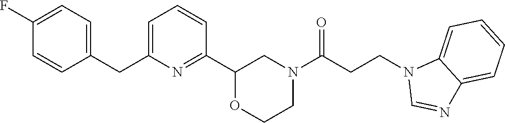 [SMILES: O=C(CCn1cnc2ccccc21)N1CCOC(c2cccc(Cc3ccc(F)cc3)n2)C1] | 3-(1H-benzo[d]imidazol-1-y1)-1- (2-(6-(4-fluorobenzyl)pyridin-2- yl)morpholino)propan-1-one |
| 41 | 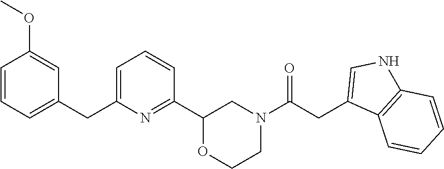 [SMILES: COc1cccc(Cc2cccc(C3CN(C(=O)Cc4c[nH]c5ccccc45)CCO3)n2)c1] | 2-(1H-indol-3-yl)-1-(2-(6-(3- methoxybenzyl)pyridin-2- yl)morpholino)ethan-1-one |
| 42 | 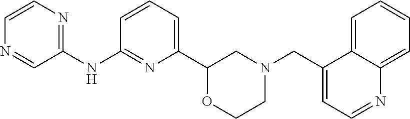 [SMILES: c1cc(Nc2cnccn2)nc(C2CN(Cc3ccnc4ccccc34)CCO2)c1] | N-(6-(4-(quinolin-4- ylmethyl)morpholin-2-yl)pyridin- 2-yl)pyrazin-2-amine |
| 43 | 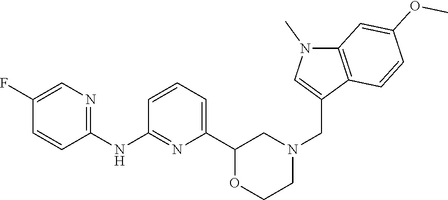 [SMILES: COc1ccc2c(CN3CCOC(c4cccc(Nc5ccc(F)cn5)n4)C3)cn(C)c2c1] | 5-fluoro-N-(6-(4-((6-methoxy-1- methyl-1H-indol-3- yl)methyl)morpholin-2- yl)pyridin-2-yl)pyridin-2-amine |
| 44 | 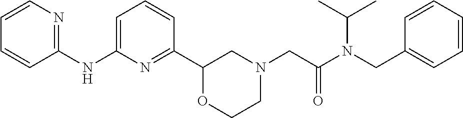 [SMILES: CC(C)N(Cc1ccccc1)C(=O)CN1CCOC(c2cccc(Nc3ccccn3)n2)C1] | N-benzyl-N-isopropyl-2-(2-(6- (pyridin-2-ylamino)pyridin-2- yl)morpholino)acetamide |
| 45 | 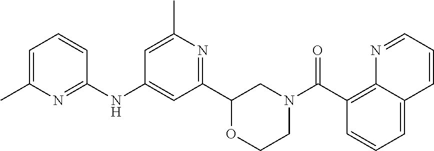 [SMILES: Cc1cccc(Nc2cc(C)nc(C3CN(C(=O)c4cccc5cccnc45)CCO3)c2)n1] | (2-(6-methyl-4-((6- methylpyridin-2- yl)amino)pyridin-2- yl)morpholino)(quinolin-8- yl)methanone |
| 46 | 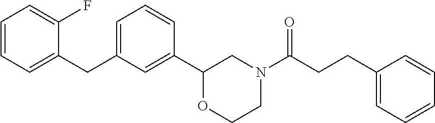 [SMILES: O=C(CCc1ccccc1)N1CCOC(c2cccc(Cc3ccccc3F)c2)C1] | 1-(2-(6-(2-fluorobenzyl)pyridin- 2-yl)morpholino)-3- phenylpropan-1-one |
| 47 | 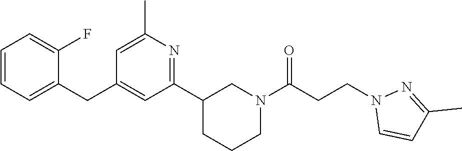 [SMILES: Cc1cc(Cc2ccccc2F)cc(C2CCCN(C(=O)CCn3ccc(C)n3)C2)n1] | 1-(3-(4-(2-fluorobenzy1)-6- methylpyridin-2-yl)piperidin-1- yl)-3-(3-methyl-1H-pyrazol-1- yl)propan-1-one |
| 48 | 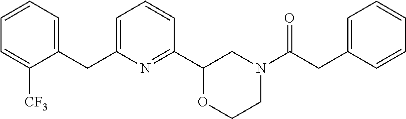 [SMILES: O=C(Cc1ccccc1)N1CCOC(c2cccc(Cc3ccccc3C(F)(F)F)n2)C1] | 2-phenyl-1-(2-(6-(2- (trifluoromethyl)benzyl)pyridin- 2-yl)morpholino)ethan-1-one |
| 49 | 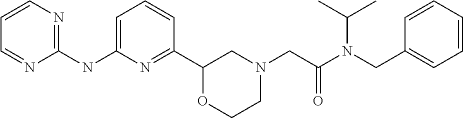 [SMILES: CC(C)N(Cc1ccccc1)C(=O)CN1CCOC(c2cccc(Nc3ncccn3)n2)C1] | N-benzyl-N-isopropyl-2-(2-(6- (pyrimidin-2-ylamino)pyridin-2- yl)morpholino)acetamide |
| 50 | 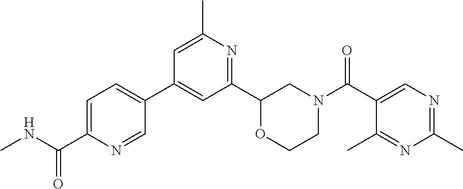 | 2′-(4-(2,4-dimethylpyrimidine-5- carbonyl)morpholin-2-yl)-N,6′- dimethy1-[3,4′-bipyridine]-6- carboxamide |
| 51 | 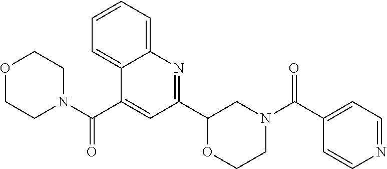 [SMILES: O=C(c1ccncc1)N1CCOC(c2cc(C(=O)N3CCOCC3)c3ccccc3n2)C1] | (2-(4-isonicotinoylmorpholin-2- yl)quinolin-4- yl)(morpholino)methanone |
| 52 | 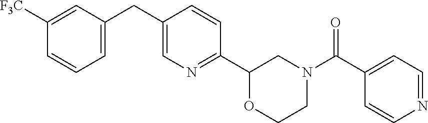 [SMILES: O=C(c1ccncc1)N1CCOC(c2ccc(Cc3cccc(C(F)(F)F)c3)cn2)C1] | pyridin-4-y1(2-(5-(3- (trifluoromethyl)benzyl)pyridin- 2-yl)morpholino)methanone |
| 53 | 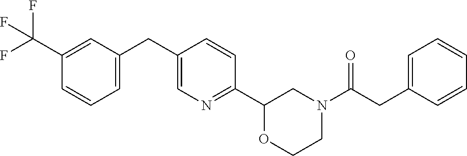 [SMILES: O=C(Cc1ccccc1)N1CCOC(c2ccc(Cc3cccc(C(F)(F)F)c3)cn2)C1] | 2-phenyl-1-(2-(5-(3- (trifluoromethyl)benzyl)pyridin- 2-yl)morpholino)ethan-1-one |
| 54 | 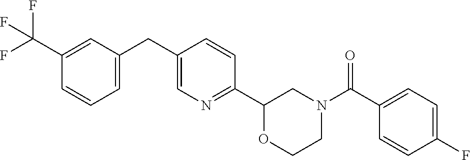 [SMILES: O=C(c1ccc(F)cc1)N1CCOC(c2ccc(Cc3cccc(C(F)(F)F)c3)cn2)C1] | (4-fluorophenyl)(2-(5-(3- (trifluoromethyl)benzyl)pyridin- 2-yl)morpholino)methanone |
| 55 | 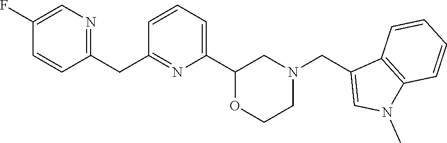 [SMILES: Cn1cc(CN2CCOC(c3cccc(Cc4ccc(F)cn4)n3)C2)c2ccccc21] | 5-fluoro-N-(6-(4-((1-methyl-1H- indol-3-yl)methyl)morpholin-2- yl)pyridin-2-yl)pyridin-2-amine |
| 56 | 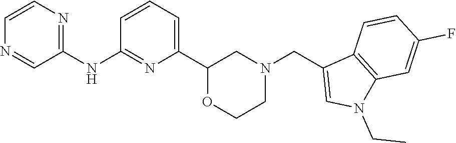 [SMILES: CCn1cc(CN2CCOC(c3cccc(Nc4cnccn4)n3)C2)c2ccc(F)cc21] | N-(6-(4-((1-ethyl-6-fluoro-1H- indol-3-yl)methyl)morpholin-2- yl)pyridin-2-yl)pyrazin-2-amine |
| 57 | 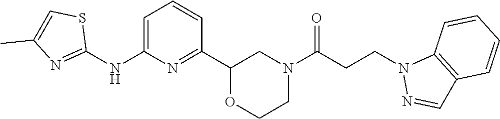 [SMILES: Cc1csc(Nc2cccc(C3CN(C(=O)CCn4ncc5ccccc54)CCO3)n2)n1] | 3-(1H-indazol-1-yl)-1-(2-(6-((4- methylthiazol-2- yl)amino)pyridin-2- yl)morpholino)propan-1-one |
| 58 | 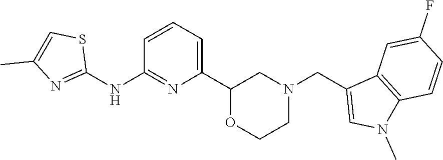 [SMILES: Cc1csc(Nc2cccc(C3CN(Cc4cn(C)c5ccc(F)cc45)CCO3)n2)n1] | N-(6-(4-((5-fluoro-1-methyl-1H- indol-3-yl)methyl)morpholin-2- yl)pyridin-2-yl)-4-methylthiazol- 2-amine |
| 59 | 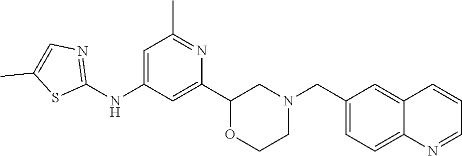 [SMILES: Cc1cc(Nc2ncc(C)s2)cc(C2CN(Cc3ccc4ncccc4c3)CCO2)n1] | 5-methyl-N-(2-methyl-6-(4- (quinolin-6-ylmethyl)morpholin- 2-yl)pyridin-4-yl)thiazol-2-amine |
| 60 | 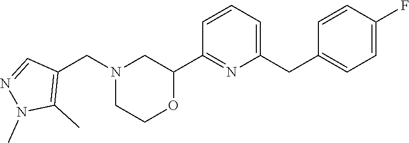 [SMILES: Cc1c(CN2CCOC(c3cccc(Cc4ccc(F)cc4)n3)C2)cnn1C] | 4-((1,5-dimethyl-1H-pyrazol-4- yl)methyl)-2-(6-(4- fluorobenzyl)pyridin-2- yl)morpholine |
| 61 | 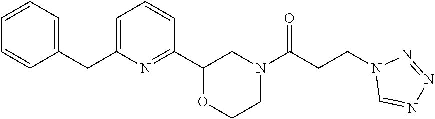 [SMILES: O=C(CCn1cnnn1)N1CCOC(c2cccc(Cc3ccccc3)n2)C1] | 1-(2-(6-benzylpyridin-2- yl)morpholino)-3-(1H-tetrazol-1- yl)propan-1-one |
| 62 | 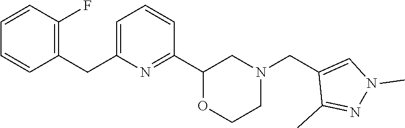 [SMILES: Cc1nn(C)cc1CN1CCOC(c2cccc(Cc3ccccc3F)n2)C1] | 4-((1,3-dimethyl-1H-pyrazol-4- yl)methyl)-2-(6-(2- fluorobenzyl)pyridin-2- yl)morpholine |
| 63 | 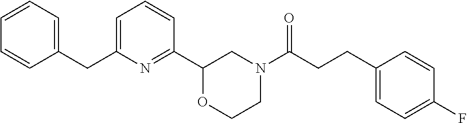 [SMILES: O=C(CCc1ccc(F)cc1)N1CCOC(c2cccc(Cc3ccccc3)n2)C1] | 1-(2-(6-benzylpyridin-2- yl)morpholino)-3-(4- fluorophenyl)propan-1-one |
| 64 | 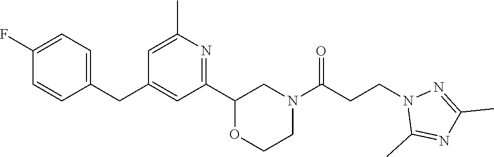 [SMILES: Cc1cc(Cc2ccc(F)cc2)cc(C2CN(C(=O)CCn3nc(C)nc3C)CCO2)n1] | 3-(3,5-dimethy1-1H-1,2,4-triazol- 1-yl)-1-(2-(4-(4-fluorobenzyl)-6- methylpyridin-2- yl)morpholino)propan-1-one |
| 65 | 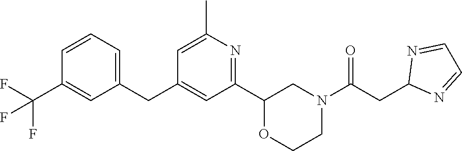 [SMILES: Cc1cc(Cc2cccc(C(F)(F)F)c2)cc(C2CN(C(=O)CC3N=CC=N3)CCO2)n1] | 1-(2-(6-methyl-4-(3- (trifluoromethyl)benzyl)pyridin- 2-yl)morpholino)-2-(1H-pyrazol- 1-yl)ethan-1-one |
| 66 | 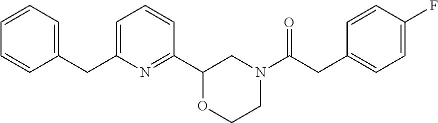 [SMILES: O=C(Cc1ccc(F)cc1)N1CCOC(c2cccc(Cc3ccccc3)n2)C1] | 1-(2-(6-benzylpyridin-2- yl)morpholino)-2-(4- fluorophenyl)ethan-1-one |
| 67 | 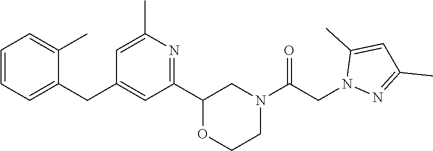 [SMILES: Cc1cc(Cc2ccccc2C)cc(C2CN(C(=O)Cn3nc(C)cc3C)CCO2)n1] | 2-(3,5-dimethyl-1H-pyrazol-1- yl)-1-(2-(6-methyl-4-(2- methylbenzyl)pyridin-2- yl)morpholino)ethan-1-one |
| 68 | 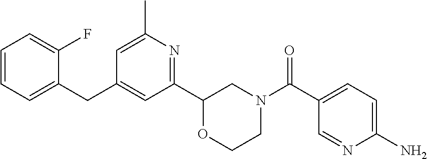 [SMILES: Cc1cc(Cc2ccccc2F)cc(C2CN(C(=O)c3ccc(N)nc3)CCO2)n1] | (6-aminopyridin-3-yl)(2-(4-(2- fluorobenzyl)-6-methylpyridin-2- yl)morpholino)methanone |
| 69 | 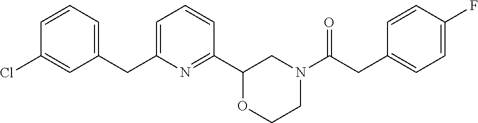 [SMILES: O=C(Cc1ccc(F)cc1)N1CCOC(c2cccc(Cc3cccc(Cl)c3)n2)C1] | 1-(2-(6-(3-chlorobenzyl)pyridin- 2-yl)morpholino)-2-(4- fluorophenyl)ethan-1-one |
| 70 | 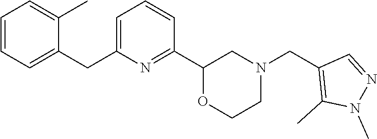 [SMILES: Cc1ccccc1Cc1cccc(C2CN(Cc3cnn(C)c3C)CCO2)n1] | 4-((1,5-dimethyl-1H-pyrazol-4- yl)methyl)-2-(6-(2- methylbenzyl)pyridin-2- yl)morpholine |
| 71 | 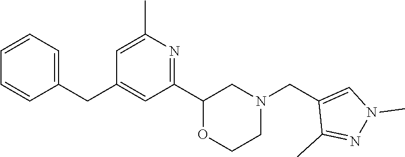 [SMILES: Cc1cc(Cc2ccccc2)cc(C2CN(Cc3cn(C)nc3C)CCO2)n1] | 2-(4-benzy1-6-methylpyridin-2- yl)-4-((1,3-dimethyl-1H-pyrazol- 4-yl)methyl)morpholine |
| 72 | 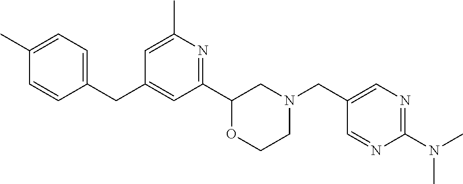 | N,N-dimethy1-5-((2-(6-methyl-4- (4-methylbenzyl)pyridin-2- yl)morpholino)methyl)pyrimidin- 2-amine |
| 73 | 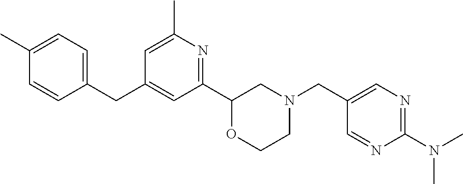 | N,N-dimethyl-5-((2-(6-methyl-4- (4-methylbenzyl)pyridin-2- yl)morpholino)methyl)pyrimidin- 2-amine |
| 74 | 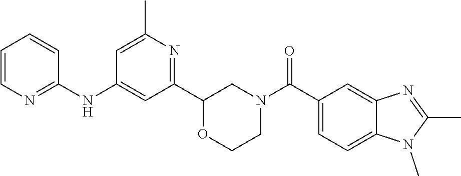 [SMILES: Cc1cc(Nc2ccccn2)cc(C2CN(C(=O)c3ccc4c(c3)nc(C)n4C)CCO2)n1] | (1,2-dimethyl-1H- benzo[d]imidazol-5-yl)(2-(6- methyl-4-(pyridin-2- ylamino)pyridin-2- yl)morpholino)methanone |
| 75 | 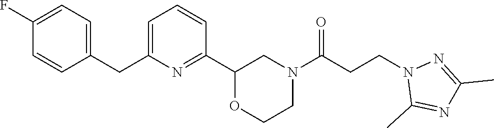 [SMILES: Cc1nc(C)n(CCC(=O)N2CCOC(c3cccc(Cc4ccc(F)cc4)n3)C2)n1] | 3-(3,5-dimethy1-1H-1,2,4-triazol- 1-yl)-1-(2-(6-(4- fluorobenzyl)pyridin-2- yl)morpholino)propan-1-one |
| 76 | 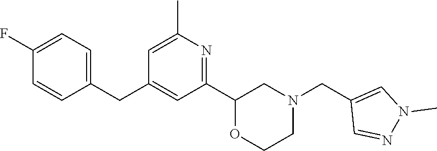 [SMILES: Cc1cc(Cc2ccc(F)cc2)cc(C2CN(Cc3cnn(C)c3)CCO2)n1] | 2-(4-(4-fluorobenzyl)-6- methylpyridin-2-yl)-4-((1-methyl- 1H-pyrazol-4- yl)methyl)morpholine |
| 77 | 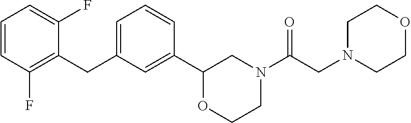 [SMILES: O=C(CN1CCOCC1)N1CCOC(c2cccc(Cc3c(F)cccc3F)c2)C1] | 1-(2-(6-(2,6- difluorobenzyl)pyridin-2- yl)morpholino)-2- morpholinoethan-1-one |
| 78 | 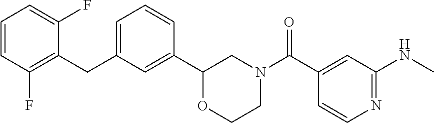 [SMILES: CNc1cc(C(=O)N2CCOC(c3cccc(Cc4c(F)cccc4F)c3)C2)ccn1] | (2-(6-(2,6-difluorobenzyl)pyridin- 2-yl)morpholino)(2- (methylamino)pyridin-4- yl)methanone |
| 79 | 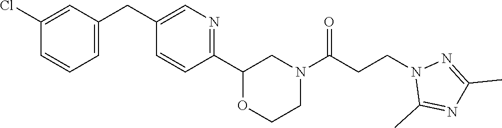 [SMILES: Cc1nc(C)n(CCC(=O)N2CCOC(c3ccc(Cc4cccc(Cl)c4)cn3)C2)n1] | 1-(2-(5-(3-chlorobenzyl)pyridin- 2-yl)morpholino)-3-(3,5- dimethy1-1H-1,2,4-triazol-1- yl)propan-1-one |
| 80 | 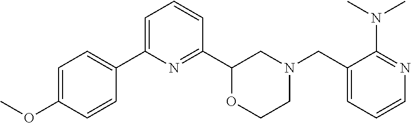 | 3-((2-(6-(4- methoxyphenyl)pyridin-2- yl)morpholino)methyl)-N,N- dimethylpyridin-2-amine |
| 81 | 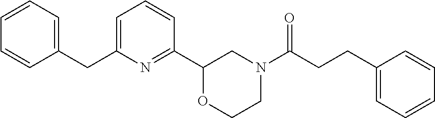 [SMILES: O=C(CCc1ccccc1)N1CCOC(c2cccc(Cc3ccccc3)n2)C1] | 1-(2-(6-benzylpyridin-2- yl)morpholino)-3-phenylpropan- 1-one |
| 82 | 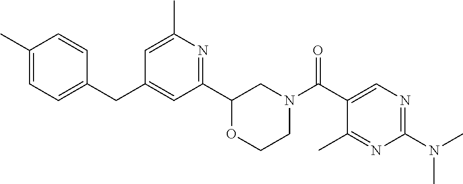 | (2-(dimethylamino)-4- methylpyrimidin-5-yl)(2-(6- methyl-4-(4- methylbenzyl)pyridin-2- yl)morpholino)methanone |
| 83 | 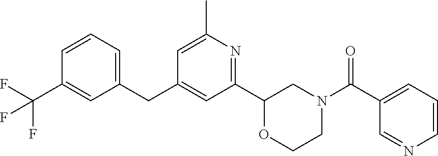 [SMILES: Cc1cc(Cc2cccc(C(F)(F)F)c2)cc(C2CN(C(=O)c3cccnc3)CCO2)n1] | (2-(6-methyl-4-(3- (trifluoromethyl)benzyl)pyridin- 2-yl)morpholino)(pyridin-3- yl)methanone |
| 84 | 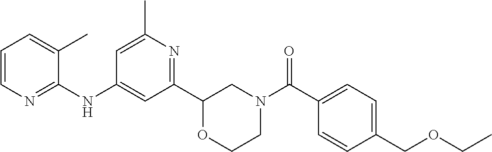 [SMILES: CCOCc1ccc(C(=O)N2CCOC(c3cc(Nc4ncccc4C)cc(C)n3)C2)cc1] | (4-(ethoxymethyl)phenyl)(2-(6- methyl-4-((3-methylpyridin-2- yl)amino)pyridin-2- yl)morpholino)methanone |
| 85 | 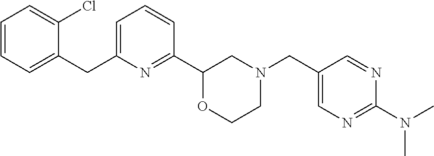 [SMILES: CN(C)c1ncc(CN2CCOC(c3cccc(Cc4ccccc4Cl)n3)C2)cn1] | 5-((2-(6-(2-chlorobenzyl)pyridin- 2-yl)morpholino)methyl)-N,N- dimethylpyrimidin-2-amine |
| 86 | 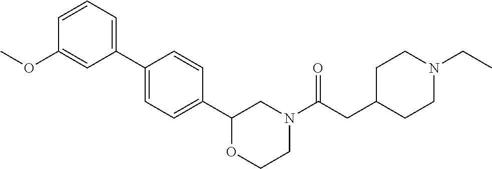 [SMILES: CCN1CCC(CC(=O)N2CCOC(c3ccc(-c4cccc(OC)c4)cc3)C2)CC1] | 2-(1-ethylpiperidin-4-yl)-1-(2-(3′- methoxy-[1,1′-biphenyl]-4- yl)morpholino)ethan-1-one |
| 87 | 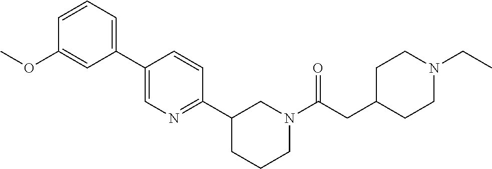 [SMILES: CCN1CCC(CC(=O)N2CCCC(c3ccc(-c4cccc(OC)c4)cn3)C2)CC1] | 2-(1-ethylpiperidin-4-y1)-1-(3-(5- (3-methoxyphenyl)pyridin-2- yl)piperidin-1-yl)ethan-1-one |
| 88 | 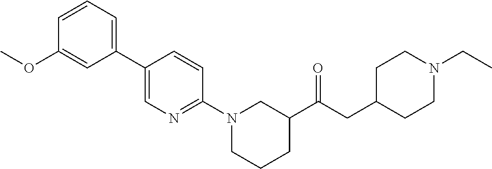 [SMILES: CCN1CCC(CC(=O)C2CCCN(c3ccc(-c4cccc(OC)c4)cn3)C2)CC1] | 2-(1-ethylpiperidin-4-y1)-1-(1-(5- (3-methoxyphenyl)pyridin-2- yl)piperidin-3-yl)ethan-1-one |
| 89 | 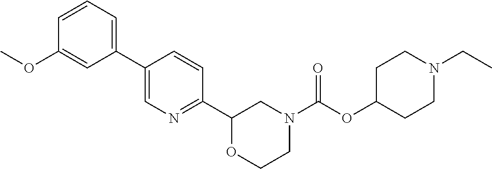 [SMILES: CCN1CCC(OC(=O)N2CCOC(c3ccc(-c4cccc(OC)c4)cn3)C2)CC1] | 1-ethylpiperidin-4-y1 2-(5-(3- methoxyphenyl)pyridin-2- yl)morpholine-4-carboxylate |
| 90 | 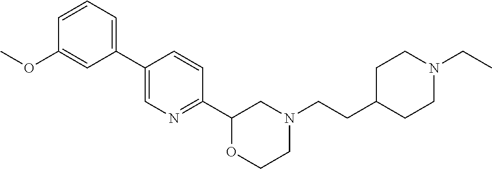 [SMILES: CCN1CCC(CCN2CCOC(c3ccc(-c4cccc(OC)c4)cn3)C2)CC1] | 4-(2-(1-ethylpiperidin-4-yl)ethyl)- 2-(5-(3-methoxyphenyl)pyridin- 2-yl)morpholine |
| 91 | 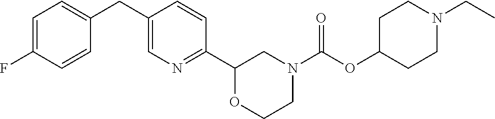 [SMILES: CCN1CCC(OC(=O)N2CCOC(c3ccc(Cc4ccc(F)cc4)cn3)C2)CC1] | 2-(1-ethylpiperidin-4-yl)-1-(2-(5- (4-fluorobenzyl)pyridin-2- yl)morpholino)ethan-1-one |
| 92 | 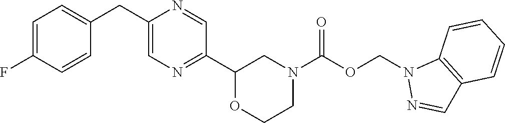 [SMILES: O=C(OCn1ncc2ccccc21)N1CCOC(c2cnc(Cc3ccc(F)cc3)cn2)C1] | 1-(2-(5-(4-fluorobenzyl)pyrazin- 2-yl)morpholino)-3-(1H-indazol- 1-yl)propan-1-one |
| 93 | 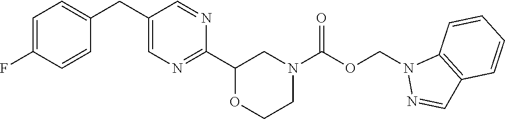 [SMILES: O=C(OCn1ncc2ccccc21)N1CCOC(c2ncc(Cc3ccc(F)cc3)cn2)C1] | 1-(2-(5-(4- fluorobenzyl)pyrimidin-2- yl)morpholino)-3-(1H-indazol-1- yl)propan-1-one |
| 94 | 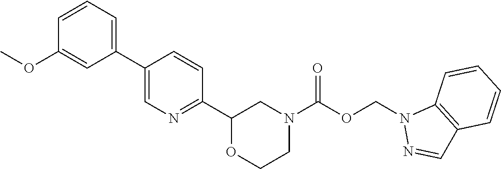 [SMILES: COc1cccc(-c2ccc(C3CN(C(=O)OCn4ncc5ccccc54)CCO3)nc2)c1] | 3-(1H-indazol-1-yl)-1-(2-(5-(3- methoxyphenyl)pyridin-2- yl)morpholino)propan-1-one |
| 95 | 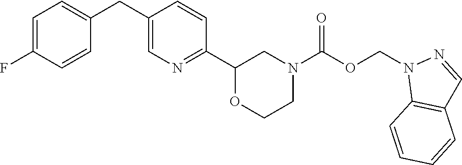 [SMILES: O=C(OCn1ncc2ccccc21)N1CCOC(c2ccc(Cc3ccc(F)cc3)cn2)C1] | 1-(2-(5-(4- fluorophenoxy)pyridin-2- yl)morpholino)-3-(1H-indazol-1- yl)propan-1-one |
| 96 | 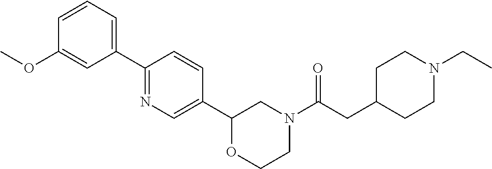 [SMILES: CCN1CCC(CC(=O)N2CCOC(c3ccc(-c4cccc(OC)c4)nc3)C2)CC1] | 2-(1-ethylpiperidin-4-yl)-1-(2-(6- (3-methoxyphenyl)pyridin-3- yl)morpholino)ethan-1-one |
| 97 | 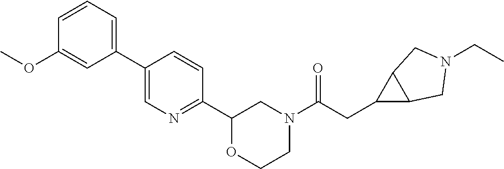 [SMILES: CCN1CC2C(CC(=O)N3CCOC(c4ccc(-c5cccc(OC)c5)cn4)C3)C2C1] | 2-(3-ethyl-3- azabicyclo[3.1.0]hexan-6-yl)-1- (2-(5-(3-methoxyphenyl)pyridin- 2-yl)morpholino)ethan-1-one |
| 98 | 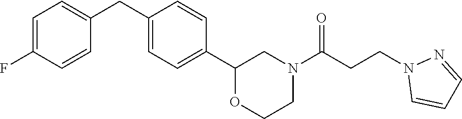 [SMILES: O=C(CCn1cccn1)N1CCOC(c2ccc(Cc3ccc(F)cc3)cc2)C1] | 1-(2-(4-(4- fluorobenzyl)phenyl)morpholino)- 3-(1H-pyrazol-1-yl)propan-1-one |
| 99 | 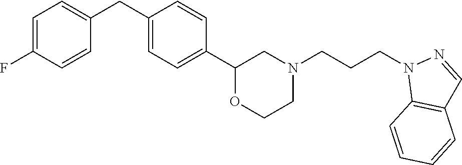 [SMILES: Fc1ccc(Cc2ccc(C3CN(CCCn4ncc5ccccc54)CCO3)cc2)cc1] | 4-(3-(1H-indazol-1-yl)propyl)-2- (4-(4- fluorobenzyl)phenyl)morpholine |
| 100 | 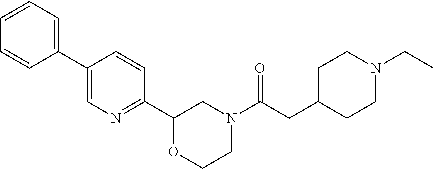 [SMILES: CCN1CCC(CC(=O)N2CCOC(c3ccc(-c4ccccc4)cn3)C2)CC1] | 2-(1-ethylpiperidin-4-yl)-1-(2-(5- phenylpyridin-2- yl)morpholino)ethan-1-one |
| 101 | 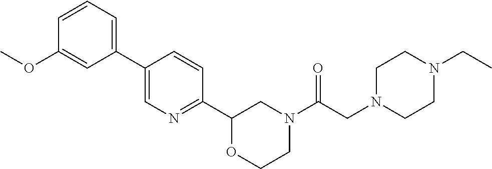 [SMILES: CCN1CCN(CC(=O)N2CCOC(c3ccc(-c4cccc(OC)c4)cn3)C2)CC1] | 2-(4-ethylpiperazin-1-yl)-1-(2-(5- (3-methoxyphenyl)pyridin-2- yl)morpholino)ethan-1-one |
| 102 | 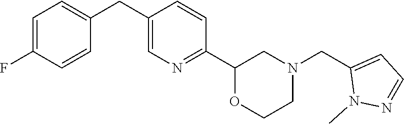 [SMILES: Cn1nccc1CN1CCOC(c2ccc(Cc3ccc(F)cc3)cn2)C1] | 2-(5-(4-fluorobenzyl)pyridin-2- yl)-4-((1-methyl-1H-pyrazol-5- yl)methyl)morpholine |
| 103 | 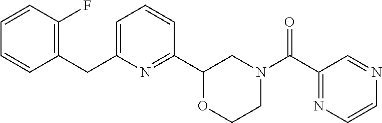 [SMILES: O=C(c1cnccn1)N1CCOC(c2cccc(Cc3ccccc3F)n2)C1] | (2-(6-(2-fluorobenzyl)pyridin-2- yl)morpholino)(pyrazin-2- yl)methanone |
| 104 | 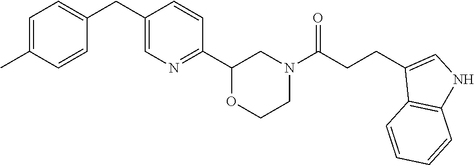 [SMILES: Cc1ccc(Cc2ccc(C3CN(C(=O)CCc4c[nH]c5ccccc45)CCO3)nc2)cc1] | 3-(1H-indol-3-yl)-1-(2-(5-(4- methylbenzyl)pyridin-2- yl)morpholino)propan-1-one |
| 105 | 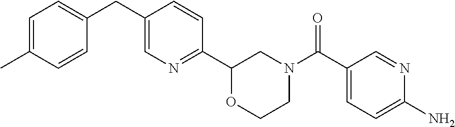 [SMILES: Cc1ccc(Cc2ccc(C3CN(C(=O)c4ccc(N)nc4)CCO3)nc2)cc1] | (6-aminopyridin-3-yl)(2-(5-(4- methylbenzyl)pyridin-2- yl)morpholino)methanone |
| 106 | 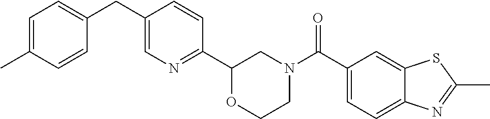 | (2-methylbenzo[d]thiazol-6-yl)(2- (5-(4-methylbenzyl)pyridin-2- yl)morpholino)methanone |
| 107 | 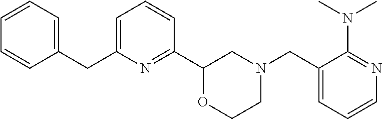 [SMILES: CN(C)c1ncccc1CN1CCOC(c2cccc(Cc3ccccc3)n2)C1] | 3-((2-(6-benzylpyridin-2- yl)morpholino)methyl)-N,N- dimethylpyridin-2-amine |
| 108 | 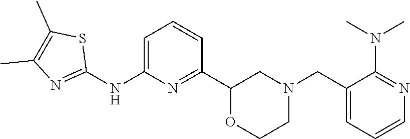 | N-(6-(4-((2- (dimethylamino)pyridin-3- yl)methyl)morpholin-2- yl)pyridin-2-yl)-4,5- dimethylthiazol-2-amine |
| 109 | 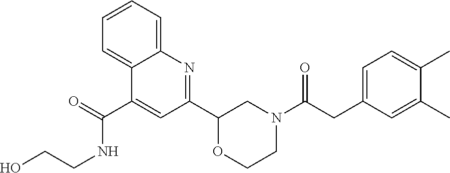 [SMILES: Cc1ccc(CC(=O)N2CCOC(c3cc(C(=O)NCCO)c4ccccc4n3)C2)cc1C] | 2-(4-(2-(3,4- dimethylphenyl)acetyl)morpholin- 2-yl)-N-(2- hydroxyethyl)quinoline-4- carboxamide |
| 110 | 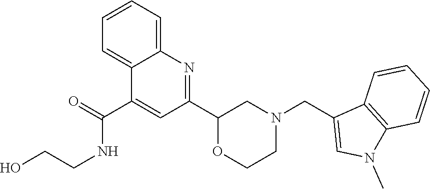 [SMILES: Cn1cc(CN2CCOC(c3cc(C(=O)NCCO)c4ccccc4n3)C2)c2ccccc21] | N-(2-hydroxyethyl)-2-(4-((1- methyl-1H-indol-3- yl)methyl)morpholin-2- yl)quinoline-4-carboxamide |
| 111 | 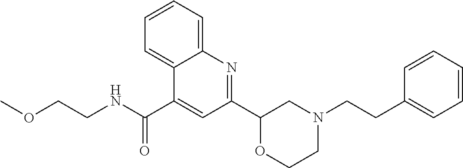 [SMILES: COCCNC(=O)c1cc(C2CN(CCc3ccccc3)CCO2)nc2ccccc12] | N-(2-methoxyethyl)-2-(4- phenethylmorpholin-2- yl)quinoline-4-carboxamide |
| 112 | 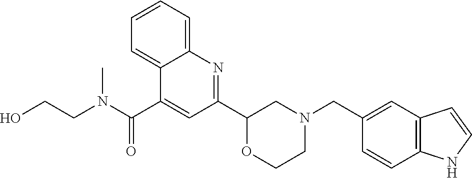 [SMILES: CN(CCO)C(=O)c1cc(C2CN(Cc3ccc4[nH]ccc4c3)CCO2)nc2ccccc12] | 2-(4-((1H-indol-5- yl)methyl)morpholin-2-yl)-N-(2- hydroxyethyl)-N- methylquinoline-4-carboxamide |
| 113 | 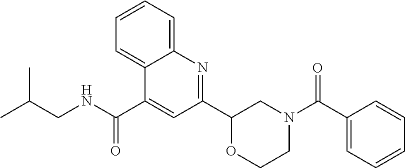 [SMILES: CC(C)CNC(=O)c1cc(C2CN(C(=O)c3ccccc3)CCO2)nc2ccccc12] | 2-(4-benzoylmorpholin-2-yl)-N- isobutylquinoline-4-carboxamide |
| 114 | 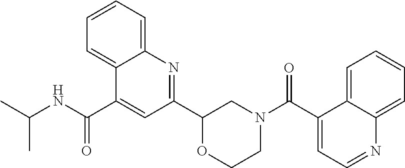 [SMILES: CC(C)NC(=O)c1cc(C2CN(C(=O)c3ccnc4ccccc34)CCO2)nc2ccccc12] | N-isopropyl-2-(4-(quinolin-4- ylmethyl)morpholin-2- yl)quinoline-4-carboxamide |
| 115 | 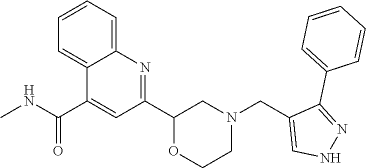 [SMILES: CNC(=O)c1cc(C2CN(Cc3c[nH]nc3-c3ccccc3)CCO2)nc2ccccc12] | N-methyl-2-(4-((3-phenyl-1H- pyrazol-4-yl)methyl)morpholin-2- yl)quinoline-4-carboxamide |
| 116 | 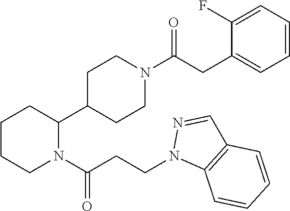 [SMILES: O=C(Cc1ccccc1F)N1CCC(C2CCCCN2C(=O)CCn2ncc3ccccc32)CC1] | 1-(1′-(2-(2-fluorophenyl)acetyl)- [2,4′-bipiperidin]-1-yl)-3-(1H- indazol-1-yl)propan-1-one |
| 117 | 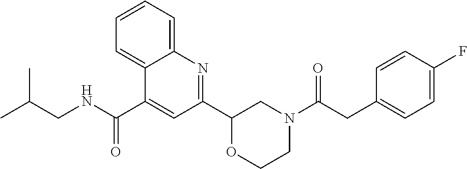 [SMILES: CC(C)CNC(=O)c1cc(C2CN(C(=O)Cc3ccc(F)cc3)CCO2)nc2ccccc12] | 2-(4-(2-(4- fluorophenyl)acetyl)morpholin-2- yl)-N-isobutylquinoline-4- carboxamide |
| 118 | 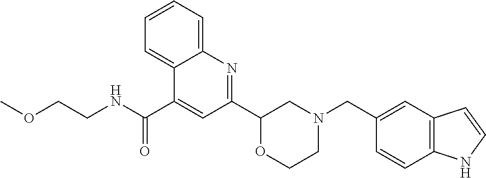 [SMILES: COCCNC(=O)c1cc(C2CN(Cc3ccc4[nH]ccc4c3)CCO2)nc2ccccc12] | 2-(4-((1H-indol-5- yl)methyl)morpholin-2-yl)-N-(2- methoxyethyl)quinoline-4- carboxamide |
| 119 | 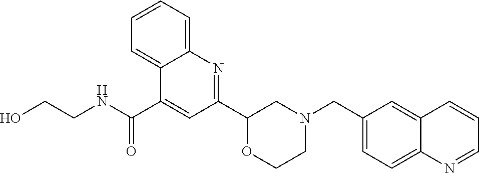 [SMILES: O=C(NCCO)c1cc(C2CN(Cc3ccc4ncccc4c3)CCO2)nc2ccccc12] | N-(2-hydroxyethyl)-2-(4- (quinolin-6-ylmethyl)morpholin- 2-yl)quinoline-4-carboxamide |
| 120 | 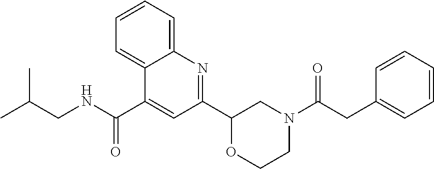 [SMILES: CC(C)CNC(=O)c1cc(C2CN(C(=O)Cc3ccccc3)CCO2)nc2ccccc12] | N-isobutyl-2-(4-(2- phenylacetyl)morpholin-2- yl)quinoline-4-carboxamide |
| 121 | 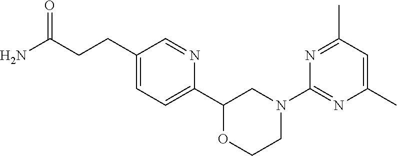 [SMILES: Cc1cc(C)nc(N2CCOC(c3ccc(CCC(N)=O)cn3)C2)n1] | 3-(6-(4-(4,6-dimethylpyrimidin- 2-yl)morpholin-2-yl)pyridin-3- yl)propanamide |
| 122 | 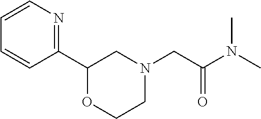 [SMILES: CN(C)C(=O)CN1CCOC(c2ccccn2)C1] | N,N-dimethyl-2-(2-(pyridin-2- yl)morpholino)acetamide |
| 123 | 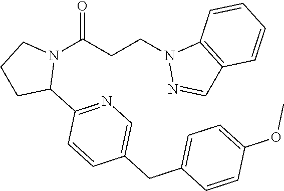 [SMILES: COc1ccc(Cc2ccc(C3CCCN3C(=O)CCn3ncc4ccccc43)nc2)cc1] | 3-(1H-indazol-1-yl)-1-(2-(5-(4- methoxybenzyl)pyridin-2- yl)pyrrolidin-1-yl)propan-1-one |
| 124 | 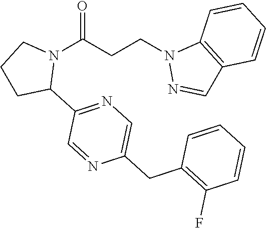 [SMILES: O=C(CCn1ncc2ccccc21)N1CCCC1c1cnc(Cc2ccccc2F)cn1] | 1-(2-(6-(3-fluorobenzyl)pyrazin- 2-yl)pyrrolidin-1-yl)-3-(1H- indazol-1-yl)propan-1-one |
| 125 | 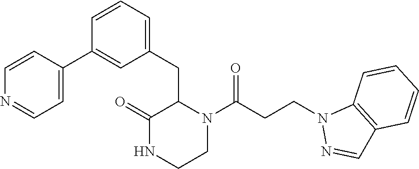 [SMILES: O=C1NCCN(C(=O)CCn2ncc3ccccc32)C1Cc1cccc(-c2ccncc2)c1] | 4-(3-(1H-indazol-1- yl)propanoyl)-3-(3-(pyridin-4- yl)benzyl)piperazin-2-one |
| 126 | 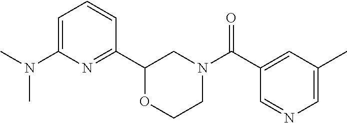 | (2-(6-(dimethylamino)pyridin-2- yl)morpholino)(5-methylpyridin- 3-y1)methanone |
| 127 | 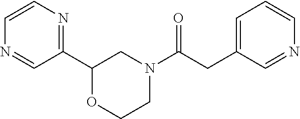 [SMILES: O=C(Cc1cccnc1)N1CCOC(c2cnccn2)C1] | 1-(2-(pyrazin-2-yl)morpholino)- 2-(pyridin-3-yl)ethan-1-one |
| 128 | 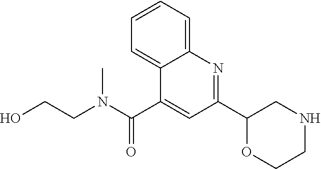 [SMILES: CN(CCO)C(=O)c1cc(C2CNCCO2)nc2ccccc12] | N-(2-hydroxyethyl)-N-methyl-2- (morpholin-2-yl)quinoline-4- carboxamide |

### Example 3: Inhibition of AKT and/or MLC Phosphorylation

Compounds 1-3 were evaluated for inhibition of AKT and MLC phosphorylation. As described elsewhere herein, phosphorylation of AKT and/or MLC can occur with activation of the Gα₁₂ pathway. Accordingly, in certain embodiments, detection of a decrease in pAKT and/or pMLC indicates inhibition of Gα₁₂.

The results indicate that pAKT and pMLC are decreased with administration of compound 1 at 100 μM. Further, compound 3 significantly decreased pAKT and pMLC at 10 μM and 100 μM.

### Example 4: Evidence Showing Target Engagement and Efficacy

Analogues of the BDF parent molecule comprising modified and/or alternate substituents of the core moiety were tested and compared with the parent molecule for inhibition of Gα₁₂ and agonist-induced airway smooth muscle (ASM) contraction using the platforms described elsewhere herein (i.e., hTERT reporter assay and immunoblot analysis).

The immunoblot analysis of phosphorylation of myosin light chain (pMLC) was used as a surrogate for ASM contraction. In parallel, phosphorylation of AKT was analogously assessed. AKT is a downstream substrate for phosphoinositide 3-kinase (PI3K) activity, can modulate the activation of the Gα₁₂ pathway in primary human airway smooth muscle cells. These cells are the pivotal cells regulating bronchomotor tone in asthma and chronic obstructive pulmonary disease (COPD) which is emphysema or chronic bronchitis.

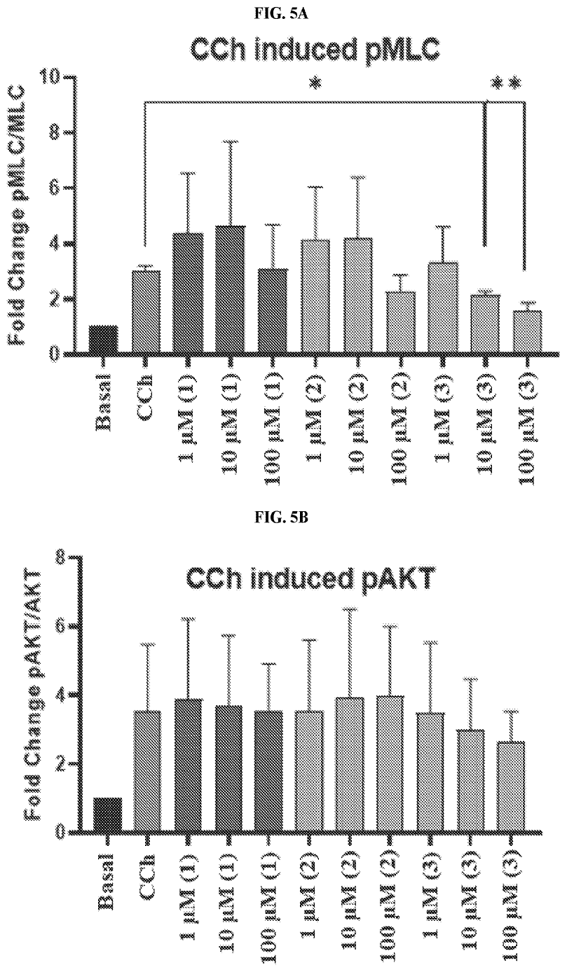

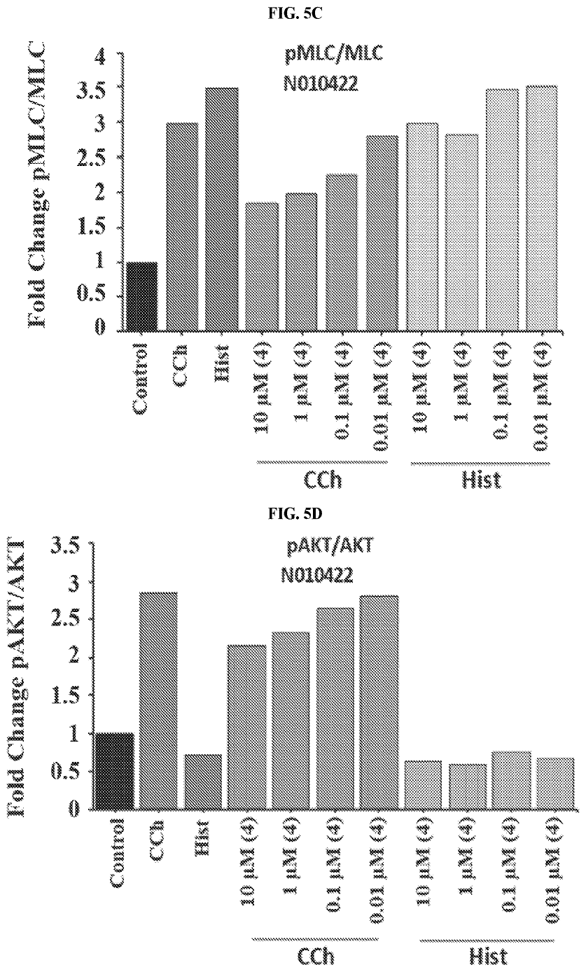

The analogues described herein were further assessed for potency and efficacy in inhibiting AKT and pMLC phosphorylation induced by contractile agonists, carbachol, or histamine. These contractile agonists are pivotal in causing airway smooth muscle contraction and airway obstruction in asthma and COPD. As depicted in FIGS. 5A-5D, compounds 2-4 effectively decreased carbachol-induced pMLC as compared with a diluent control or the parent compound (1).

Each of compounds 2-5, however, are ineffective in inhibiting AKT phosphorylation by agonists. Without wishing to be limited by any theory, these data suggest that carbachol rather than histamine activates Gα₁₂ and that the inhibition of Gα₁₂-mediated ASM contraction may or may not require activation of AKT. Collectively, these data inform that certain modifications of the parent BDF molecule (i.e., compound 1), for example, as represented in compounds 2 and 3, afford compounds with improved potency and efficacy than the parent compound on carbachol-induced pMLC activation and these effects are specific for carbachol rather than histamine.

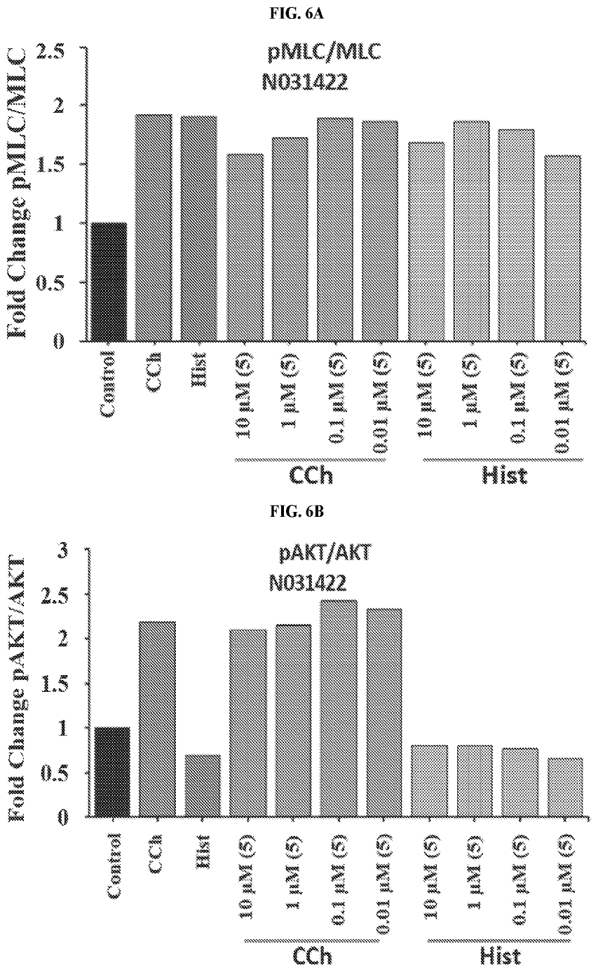

In contrast, a side chain modification generating compound 5 (H2) was inactive for agonist-induced pAKT and pMLC activation as shown in (FIGS. 6A-6B).

Thus, in one aspect, the present disclosure describes certain compounds which are potent and efficacious inhibitors of specific agonist-induced contractile responses in human ASM cells.

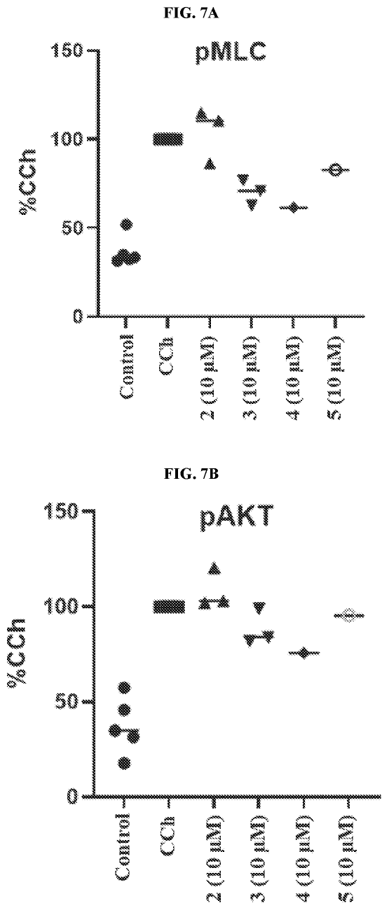

FIGS. 7A-7B shows a summary of such data using multiple ASM cell lines with a minimum of three technical replicates. These studies provide evidence that inhibiting Gα₁₂ can serve as an approach for use of bronchodilators in asthma and COPD.

### Enumerated Embodiments

The following exemplary embodiments are provided, the numbering of which is not to be construed as designating levels of importance:

Embodiment 1 provides a method of treating, preventing, and/or ameliorating an airway disease in a subject, the method comprising administering to the subject a compound of formula (I), or a salt, solvate, prodrug, stereoisomer, isotopologue, or tautomer thereof, or any mixtures thereof:

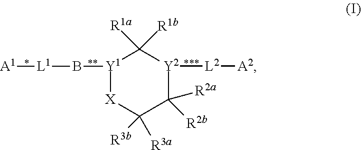

wherein:
A¹ is selected from the group consisting of optionally substituted phenyl and optionally substituted C₁-C₁₀ heterocyclyl;
A² is selected from the group consisting of optionally substituted phenyl, optionally substituted C₁-C₁₀ heterocyclyl, and NH (optionally substituted C₂-C₈ heterocyclyl);
B is

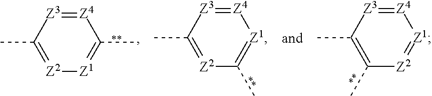

L¹ is selected from the group consisting of a bond. —NR⁴—, —O—, and optionally substituted C₁-C₃ alkylenyl;
L² is selected from the group consisting of a bond, —C(═O)—, —C(═O)O—, —C(═O)(optionally substituted C₁-C₃ alkylenyl)-, and optionally substituted C₁-C₃ alkylenyl;
X is selected from the group consisting of —O— and —C(R^{5a})(R^{5b})—,
Y¹ and Y² are each independently selected from the group consisting of N and CR⁶,
   wherein Y¹ and Y² cannot be both N, and
   wherein if X is O, then Y¹ is CR⁶;
Z¹ is selected from the group consisting of CR⁷a and N, Z² is selected from the group consisting of CR^{7b} and N, Z³ is selected from the group consisting of CR^{7c} and N, and Z⁴ is selected from the group consisting of CR^{7d} and N,
   wherein 0-2 selected from the group consisting of Z¹, Z², Z³, and Z⁴ are N;
R^{1a}, R^{1b}, R^{2a}, R^{2b}, R^{3a}, and R^{3b} are each independently selected from the group consisting of H, optionally substituted C₁-C₆ alkyl, optionally substituted C₃-C₈ cycloalkyl, optionally substituted C₁-C₆ alkoxy, and halogen;
R⁴ is selected from the group consisting of H and optionally substituted C₁-C₆ alkyl;
R^{5a} and R^{5b} are each independently selected from the group consisting of H, optionally substituted C₁-C₆ alkyl, optionally substituted C₃-C₈ cycloalkyl, optionally substituted C₁-C₆ alkoxy, and halogen;
R⁶ is selected from the group consisting of H, optionally substituted C₁-C₆ alkyl, optionally substituted C₃-C₈ cycloalkyl, optionally substituted C₁-C₆ alkoxy, and halogen;
R^{7a}, R^{7b}, R^{7c}, and R^{7d} are each independently selected from the group consisting of H, optionally substituted C₁-C₆ alkyl, optionally substituted C₃-C₈ cycloalkyl, optionally substituted C₁-C₆ alkoxy, optionally substituted phenyl, optionally substituted C₂-C₈ heterocyclyl, halogen, CN, NO₂, OR^{a}, N(R^{a})(R^{b}), C(═O)R^{a}, C(═O)OR^{a}, OC(═O)R^{a}, C(═O)N(R^{a})(R^{b}), S(═O)₂N(R^{a})(R^{b}), NR^{a}C(═O)R^{b}, and NR^{a}S(═O)₂R^{b}, wherein two vicinal substituents selected from the group consisting of R^{7a}, R^{7b}, R^{7c}, and R^{7d} can combine with the atoms to which they are bound to form an optionally substituted phenyl or optionally substituted C₂-C₈ heterocyclyl;
R^{a} and R^{b} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl;
* indicates a bond between A¹ and L¹;
** indicates a bond between B and Y¹; and
*** indicates a bond between Y² and L².

Embodiment 2 provides the method of Embodiment 1, wherein contraction of an airway smooth muscle cell is inhibited.

Embodiment 3 provides the method of Embodiment 1 or 2, wherein dilation of an airway smooth muscle cell is promoted.

Embodiment 4 provides the method of any one of Embodiments 1-3, wherein the airway disease is at least one selected from the group consisting of asthma, chronic obstructive pulmonary disease (COPD), chronic bronchitis, bronchiectasis, and cystic fibrosis.

Embodiment 5 provides a method of inhibiting bronchoconstriction and/or promoting bronchodilation in a subject, the method comprising administering to the subject a compound of formula (I), or a salt, solvate, prodrug, stereoisomer, isotopologue, or tautomer thereof, or any mixtures thereof:

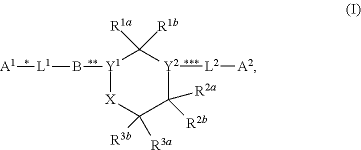

wherein:
A¹ is selected from the group consisting of optionally substituted phenyl and optionally substituted C₁-C₁₀ heterocyclyl;
A² is selected from the group consisting of optionally substituted phenyl, optionally substituted C₁-C₁₀ heterocyclyl, and NH (optionally substituted C₂-C₈ heterocylyl);
B is

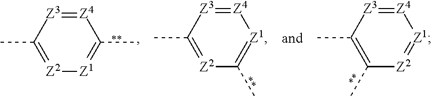

L¹ is selected from the group consisting of a bond, —NR⁴—, —O—, and optionally substituted C₁-C₃ alkylenyl;
L² is selected from the group consisting of a bond, —C(═O)—, —C(═O)O—, —C(═O)(optionally substituted C₁-C₃ alkylenyl)-, and optionally substituted C₁-C₃ alkylenyl;
X is selected from the group consisting of —O— and —C(R^{5a})(R^{5b})—,
Y¹ and Y² are each independently selected from the group consisting of N and CR⁶,
   wherein no more than one of Y¹ and Y² is N, and
   wherein if X is O, then Y¹ is CR⁶;
Z¹ is selected from the group consisting of CR^{7a} and N, Z² is selected from the group consisting of CR^{7b} and N, Z³ is selected from the group consisting of CR^{7c} and N, and Z⁴ is selected from the group consisting of CR^{7d} and N,
   wherein 0-2 selected from the group consisting of Z¹, Z², Z³, and Z⁴ are N;
R^{1a}, R^{1b}, R^{2a}, R^{2b}, R^{3a}, and R^{3b} are each independently selected from the group consisting of H, optionally substituted C₁-C₆ alkyl, optionally substituted C₃-C₈ cycloalkyl, optionally substituted C₁-C₆ alkoxy, and halogen;
R⁴ is selected from the group consisting of H and optionally substituted C₁-C₆ alkyl;
R^{5a} and R^{5b} are each independently selected from the group consisting of H, optionally substituted C₁-C₆ alkyl, optionally substituted C₃-C₈ cycloalkyl, optionally substituted C₁-C₆ alkoxy, and halogen;
R⁶ is selected from the group consisting of H, optionally substituted C₁-C₆ alkyl, optionally substituted C₃-C₈ cycloalkyl, optionally substituted C₁-C₆ alkoxy, and halogen;
R^{7a}, R^{7b}, R^{7c}, and R^{7d} are each independently selected from the group consisting of H, optionally substituted C₁-C₆ alkyl, optionally substituted C₃-C₈ cycloalkyl, optionally substituted C₁-C₆ alkoxy, optionally substituted phenyl, optionally substituted C₂-C₈ heterocyclyl, halogen, CN, NO₂, OR^{a}, N(R^{a})(R^{b}), C(═O)R^{a}, C(═O)OR^{a}, OC(═O)R^{a}, C(═O)N(R^{a})(R^{b}), S(═O)₂N(R^{a})(R^{b}), NR₂C(═O)R^{b}, and NR^{a}S(═O)₂R^{b};
   wherein two vicinal substituents selected from the group consisting of R^{7a}, R^{1b}, R^{7c}, and R^{7d} can combine with the atoms to which they are bound to form an optionally substituted phenyl or optionally substituted C₂-C₈ heterocyclyl;
R^{a} and R^{b} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl;
*indicates a bond between A¹ and L¹;
** indicates a bond between B and Y¹; and
*** indicates a bond between Y² and L².

Embodiment 6 provides the method of any one of Embodiments 1-5, wherein Gα₁₂ is inhibited.

Embodiment 7 provides the method of any one of Embodiments 1-6, further comprising administering to the subject a therapeutically effective amount of one or more additional agents.

Embodiment 8 provides the method of Embodiment 7, wherein the one or more additional agents are selected from the group consisting of a phosphoinositide 3-kinase (PI3K) inhibitor and a rho kinase (ROCK) inhibitor.

Embodiment 9 provides the method of Embodiment 8, wherein the PI3K inhibitor is selected from the group consisting of idelalisib, copanlisib, duvelisib, alpelisib, umbralisib, buparlisib, dactolisib, leniolisib, parsaclisib, paxalisib, taselisib, zandelisib, inavolisib, apitolisib, bimarlisib, eganelisib, fimepinostat, gedatolisib, linperlisib, nemiralisib, pictilisib, pilaralisib, samotolisib, seletalisib, serabelisib, sonolisib, tenalisib, voxtalisib, AMG 319, AZD8186, GSK2636771, SF1126, acalisib, omipalisib, AZD8835, CAL263, GSK1059615, MEN1611, PWT33597, TG100-115, and ZSTK474.

Embodiment 10 provides the method of Embodiment 8, wherein the ROCK inhibitor is selected from the group consisting of rhosin, AT-13148, BA-210, D-elemene, chroman 1, DJ4, fasudil, GSK-576371, GSK429286A, H-1152, hydroxyfasudil, ibuprofen, LX-7101, netarsudil, RKI-1447, ripasudil, TCS-7001, thiazovivin, verosudil, Y-27632, Y-30141, Y-33075, and Y-39983.

Embodiment 11 provides the method of any one of Embodiments 1-10, wherein the compound of formula (I) is selected from the group consisting of:

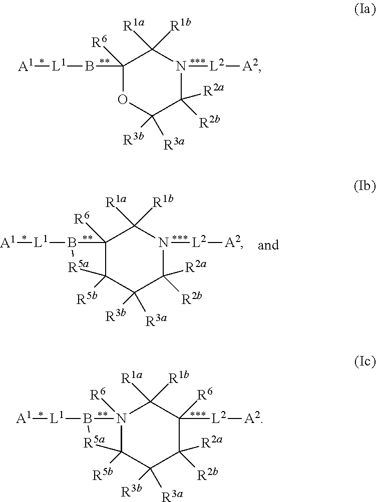

Embodiment 12 provides the method of Embodiment 11, wherein the compound of formula (I) is selected from the group consisting of

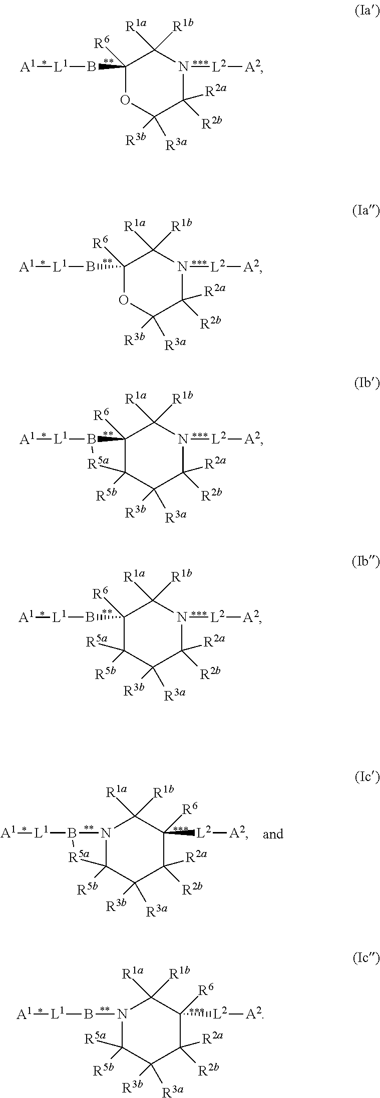

Embodiment 13 provides the method of any one of Embodiments 1-12, wherein one of the following applies:
(a) R^{1a}, R^{1b}, R^{2a}, R^{2b}, R^{3a}, and R^{3b} are each independently H;
(b) R^{1a}, R^{1b}, R^{2a}, R^{2b}, R^{3a}, R^{3b}, R^{5a}, and R^{5b} are each independently H;
(c) R^{1a}, R^{1b}, R^{2a}, R^{2b}, R^{3a}, R^{3b}, and R⁶ are each independently H; or
(d) R^{1a}, R^{1b}, R^{2a}, R^{2b}, R^{3a}, R^{3b}, R^{5a}, R^{5b}, and R⁶ are each independently H.

Embodiment 14 provides the method of any one of Embodiments 1-13, wherein B is selected from the group consisting of

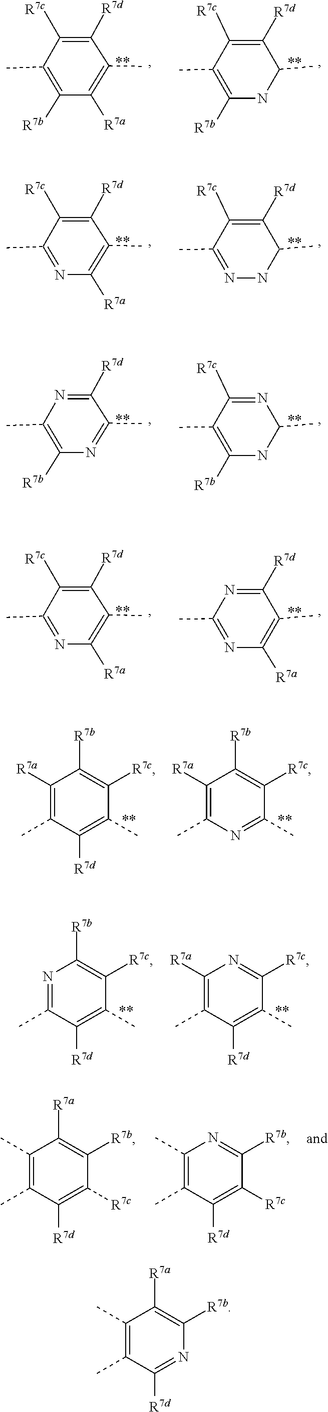

Embodiment 15 provides the method of any one of Embodiments 1-14, wherein at least one of the following applies:
(a) at least one selected from the group consisting of R^{7a}, R^{7b}, R^{7c}, and R^{7d} is H;
(b) at least two selected from the group consisting of R^{7a}, R^{7b}, R^{7c}, and R^{7d} are H;
(c) at least three selected from the group consisting of R^{7a}, R^{7b}, R^{7c}, and R^{7d} are H;
(d) each of R^{7a}, R^{7b}, R^{7c}, and R^{7d} are H; and
(e) two vicinal substituents selected from the group consisting of R^{7a}, R^{7b}, R^{7c}, and R^{7d} combine to form phenyl.

Embodiment 16 provides the method of any one of Embodiments 1-15, wherein B is selected from the group consisting of

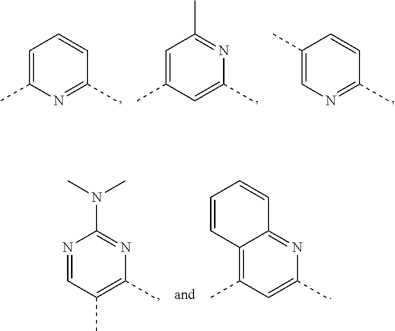

Embodiment 17 provides the method of any one of Embodiments 1-16, wherein the phenyl or heterocyclyl in A¹ is optionally substituted with at least one substituent selected from the group consisting of methyl, trifluoromethyl, fluorine, chlorine, methoxy, and C(═O)NH₂.

Embodiment 18 provides the method of any one of Embodiments 1-17, wherein A¹ is selected from the group consisting of phenyl,

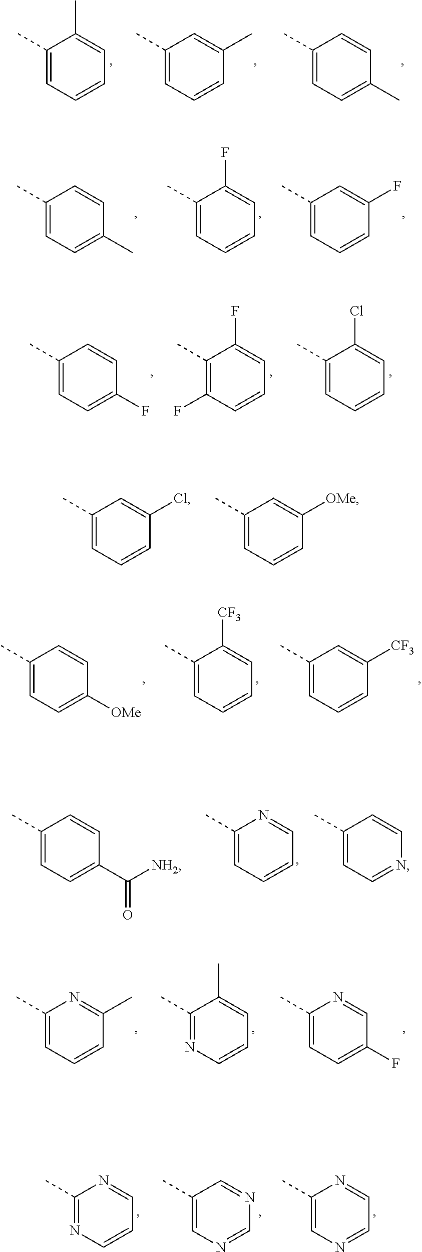

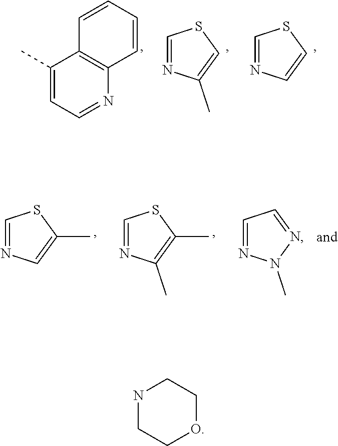

Embodiment 19 provides the method of any one of Embodiments 1-18, wherein A² is selected from the group consisting of:

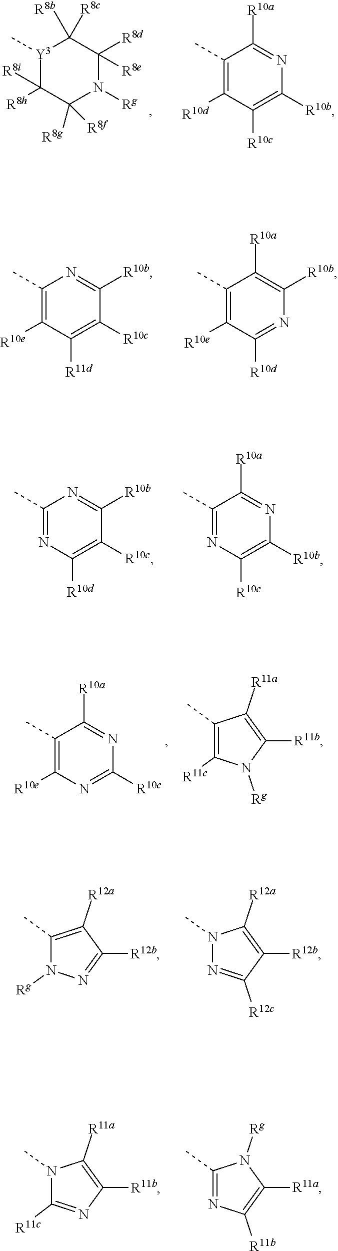

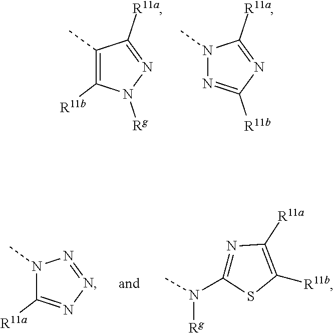

wherein:
R^{8a}, R^{8b}, R^{8c}, R^{8d}, R^{8e}, R^{8f}, R^{8g}, R^{8h}, and R⁸ⁱ are each independently selected from the group consisting of H, optionally substituted C₁-C₆ alkyl, optionally substituted C₃-C₈ cycloalkyl, and halogen,
   wherein any two substituents selected from the group consisting of R^{8a}, R^{8b}, R^{8c}, R^{8d}, R^{8e}, R^{8f}, R^{8g}, R^{8h}, and R⁸ⁱ may combine with the atoms to which they are bound to form an optionally substituted C₃-C₈ cycloalkyl or optionally substituted C₂-C₈ heterocyclyl;
R⁹ is selected from the group consisting of H, optionally substituted C₁-C₆ alkyl, optionally substituted C₁-C₆ cycloalkyl, and optionally substituted benzyl;
R^{10a}, R^{10b}, R^{10c}, R^{10d}, and R^{10e}, if present, are each independently selected from the group consisting of H, optionally substituted C₁-C₆ alkyl, optionally substituted C₃-C₈ cycloalkyl, halogen, N(R^{a})(R^{b}), CN, and NO₂,
   wherein no more than one of R^{10a}, R^{10b}, R^{10c}, R^{10d}, and R^{10e} is N(R^{a})(R^{b}), and two vicinal substituents selected from the group consisting of R^{10a}, R^{10b}, R^{10c}, R^{10d}, and R^{10e} can combine with the atoms to which they are bound to form an optionally substituted phenyl or optionally substituted C₂-C₈ heterocyclyl;
R^{11a}, R^{11b}, and R^{11c}, if present, are each independently selected from the group consisting of H, optionally substituted C₁-C₆ alkyl, optionally substituted C₃-C₈ cycloalkyl, halogen, CN, and NO₂,
   wherein two vicinal substituents selected from the group consisting of R^{11a}, R^{11b}, and R^{11c} can combine with the atoms to which they are bound to form an optionally substituted phenyl or optionally substituted C₂-C₈ heterocyclyl; and
Y³ is selected from the group consisting of R^{8a} and N.

Embodiment 20 provides the method of any one of Embodiments 1-19, wherein A² is selected from the group consisting of:

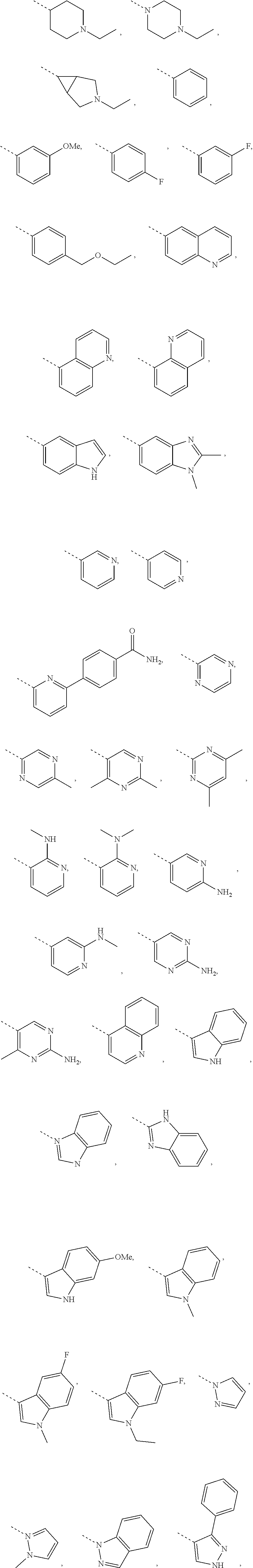

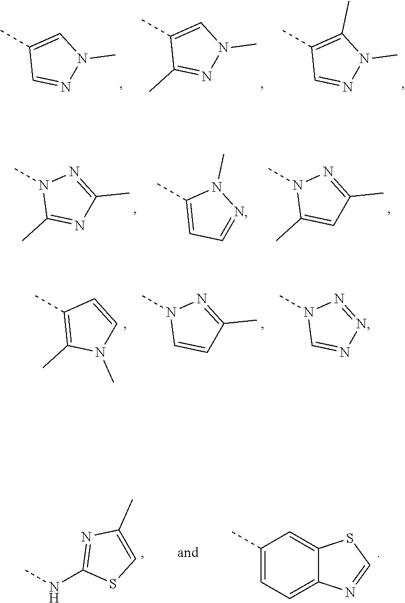

Embodiment 21 provides the method of any one of Embodiments 1-20, wherein L¹ is selected from the group consisting of a bond, —NH—, —O—, and —CH₂—.

Embodiment 22 provides the method of any one of Embodiments 1-21, wherein L² is selected from the group consisting of a bond, —CH₂—, —CH₂CH₂—, —CH₂CH₂CH₂—, —C(═O)—,

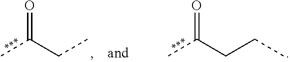

Embodiment 23 provides the method of any one of Embodiments 1-22, wherein each occurrence of optionally substituted alkyl, optionally substituted cycloalkyl, optionally substituted heterocyclyl, optionally substituted alkylenyl, optionally substituted alkoxy, optionally substituted benzyl, and optionally substituted phenyl, if present, is optionally substituted with at least one substituent selected from the group consisting of C₁-C₆ alkyl, C₁-C₆ heteroalkyl, C₃-C₈ cycloalkyl, C₁-C₆ haloalkyl, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, wherein each occurrence of R′ and R″ is independently selected from the group consisting of H, C₁-C₆ alkyl, C₃-C₈ cycloalkyl, C₁-C₆ haloalkyl, benzyl, and phenyl.

Embodiment 24 provides the method of any one of Embodiments 1-23, wherein the compound of formula (I) is selected from the group consisting of:
1-(2-(5-(4-fluorobenzyl)pyridin-2-yl)morpholino)-3-(1H-indazol-1-yl)propan-1-one;
2-(1-ethylpiperidin-4-yl)-1-(2-(5-(3-methoxyphenyl)pyridin-2-yl)morpholino)ethan-1-one;
(2-(5-(4-methylbenzyl)pyridin-2-yl)morpholino)(5-methylpyrazin-2-yl)methanone;
N-methyl-3-((2-(5-(3-(trifluoromethyl)benzyl)pyridin-2-yl)morpholino)methyl)pyridin-2-amine;
4-(pyridin-3-ylmethyl)-2-(5-(3-(trifluoromethyl)benzyl)pyridin-2-yl)morpholine;
2-(6-(4-fluorobenzyl)pyridin-2-yl)-4-(quinolin-6-ylmethyl)morpholine;
4-(6-(4-(quinolin-4-ylmethyl)morpholin-2-yl)pyridin-2-yl)benzamide;
morpholino(2-(4-phenethylmorpholin-2-yl)quinolin-4-yl)methanone;
(2-(4-(3-methoxybenzyl)morpholin-2-yl)quinolin-4-yl)(morpholino)methanone;
2-(4-(2-(3,4-dimethylphenyl)acetyl)morpholin-2-yl)-N-(2-hydroxyethyl)quinoline-4-carboxamide;
4-(6-(4-(4-(4-fluorophenyl)butanoyl)morpholin-2-yl)pyridin-3-yl)benzamide;
3-(1H-indol-3-yl)-1-(2-(6-(4-methoxyphenyl)pyridin-2-yl)morpholino)propan-1-one;
N-(2-hydroxyethyl)-2-(4-((1-methyl-1H-indol-3-yl)methyl)morpholin-2-yl)quinoline-4-carboxamide;
N-(2-methoxyethyl)-2-(4-phenethylmorpholin-2-yl)quinoline-4-carboxamide;
3-(1H-benzo[d]imidazol-2-yl)-1-(2-(6-(2-fluorobenzyl)pyridin-2-yl)morpholino)propan-1-one;
3-(1H-benzo[d]imidazol-2-yl)-1-(2-(5-(2-fluorobenzyl)pyridin-2-yl)morpholino)propan-1-one;
(2-(6-(2-chlorobenzyl)pyridin-2-yl)morpholino)(quinolin-5-yl)methanone;
(2-(6-benzylpyridin-2-yl)morpholino)(quinolin-8-yl)methanone;
2-(5-(4-methoxybenzyl)pyridin-2-yl)-4-(quinolin-5-ylmethyl)morpholine;
2-(4-benzylmorpholin-2-yl)-N-(thiazol-2-yl)quinoline-4-carboxamide;
(2-(5-(2-chlorobenzyl)pyridin-2-yl)morpholino)(quinolin-8-yl)methanone;
2-(4-((1H-indol-5-yl)methyl)morpholin-2-yl)-N-(2-hydroxyethyl)-N-methylquinoline-4-carboxamide;
2-(4-benzoylmorpholin-2-yl)-N-isobutylquinoline-4-carboxamide;
5-(4-fluorophenyl)-N,N-dimethyl-4-(4-(quinolin-4-ylmethyl)morpholin-2-yl)pyrimidin-2-amine;
1-(2-(2-(dimethylamino)-5-(3-fluorophenyl)pyrimidin-4-yl)morpholino)-2-(4-fluorophenyl)ethan-1-one;
N-isopropyl-2-(4-(quinolin-4-ylmethyl)morpholin-2-yl)quinoline-4-carboxamide;
N-methyl-2-(4-((3-phenyl-1H-pyrazol-4-yl)methyl)morpholin-2-yl)quinoline-4-carboxamide;
1-(2-(6-(2,6-difluorophenyl)pyridin-2-yl)morpholino)-3-(1H-indol-3-yl)propan-1-one;
(2-(4-benzylmorpholin-2-yl)quinolin-4-yl)(morpholino)methanone;
2-(4-(2-(4-fluorophenyl)acetyl)morpholin-2-yl)-N-isobutylquinoline-4-carboxamide;
2-(4-((1H-indol-5-yl)methyl)morpholin-2-yl)-N-(2-methoxyethyl)quinoline-4-carboxamide;
N-(2-hydroxyethyl)-2-(4-(quinolin-6-ylmethyl)morpholin-2-yl)quinoline-4-carboxamide;
phenyl(2-(5-(3-(trifluoromethyl)benzyl)pyridin-2-yl)morpholino)methanone;
1-(2-(5-(2-fluorobenzyl)pyridin-2-yl)morpholino)-3-(1H-indol-3-yl)propan-1-one;
2-(5-(4-methoxybenzyl)pyridin-2-yl)-4-(quinolin-4-ylmethyl)morpholine;
1-(2-(6-(2-fluorobenzyl)pyridin-2-yl)morpholino)-2-(3-fluorophenyl)-2-methylpropan-1-one;
N-isobutyl-2-(4-(2-phenylacetyl)morpholin-2-yl)quinoline-4-carboxamide;
1-(2-(4-(3-fluorobenzyl)-6-methylpyridin-2-yl)morpholino)-2-(4-fluorophenyl)ethan-1-one;
3-(6-(4-(4,6-dimethylpyrimidin-2-yl)morpholin-2-yl)pyridin-3-yl)propenamide;
4-((1,5-dimethyl-1H-pyrazol-4-yl)methyl)-2-(5-(pyrimidin-5-yl)pyridin-2-yl)morpholine;
(2-(6-(2-fluorobenzyl)pyridin-2-yl)morpholino)(2-(methylamino)pyridin-4-yl)methanone;
N-(6-(4-(quinolin-5-ylmethyl)morpholin-2-yl)pyridin-2-yl)pyrazin-2-amine;
N-(6-(4-((6-methoxy-1-methyl-1H-indol-3-yl)methyl)morpholin-2-yl)pyridin-2-yl)-4-methylthiazol-2-amine;
N-(6-(4-((2-(dimethylamino)pyridin-3-yl)methyl)morpholin-2-yl)pyridin-2-yl)-4-methylthiazol-2-amine;
4-methyl-N-(6-(4-(3-methylbenzyl)morpholin-2-yl)pyridin-2-yl)thiazol-2-amine;
2-(6-(4-methoxyphenyl)pyridin-2-yl)-4-(quinolin-5-ylmethyl)morpholine;
2-(6-(2-fluorobenzyl)pyridin-2-yl)-4-(quinolin-5-ylmethyl)morpholine;
2-(1H-benzo[d]imidazol-1-yl)-1-(2-(4-(2-fluorobenzyl)-6-methylpyridin-2-yl)morpholino)ethan-1-one;
morpholino(2-(4-(3-phenylpropyl)morpholin-2-yl)quinolin-4-yl)methanone;
(2-(6-(3-methoxybenzyl)pyridin-2-yl)morpholino)(quinolin-8-yl)methanone;
3-(3-((2-(6-(3-fluorophenyl)pyridin-2-yl)morpholino)methyl)-1H-indol-1-yl)propan-1-ol;
3-(1H-benzo[d]imidazol-1-yl)-1-(2-(6-(4-fluorobenzyl)pyridin-2-yl)morpholino)propan-1-one;
2-(1H-indol-3-yl)-1-(2-(6-(3-methoxybenzyl)pyridin-2-yl)morpholino)ethan-1-one;
N-(6-(4-(quinolin-4-ylmethyl)morpholin-2-yl)pyridin-2-yl)pyrazin-2-amine;
5-fluoro-N-(6-(4-((6-methoxy-1-methyl-1H-indol-3-yl)methyl)morpholin-2-yl)pyridin-2-yl)pyridin-2-amine;
N-benzyl-N-isopropyl-2-(2-(6-(pyridin-2-ylamino)pyridin-2-yl)morpholino)acetamide;
(2-(6-methyl-4-((6-methylpyridin-2-yl)amino)pyridin-2-yl)morpholino)(quinolin-8-yl)methanone;
1-(2-(6-(2-fluorobenzyl)pyridin-2-yl)morpholino)-3-phenylpropan-1-one;
1-(3-(4-(2-fluorobenzyl)-6-methylpyridin-2-yl)piperidin-1-yl)-3-(3-methyl-1H-pyrazol-1-yl)propan-1-one;
2-phenyl-1-(2-(6-(2-(trifluoromethyl)benzyl)pyridin-2-yl)morpholino)ethan-1-one;
N-benzyl-N-isopropyl-2-(2-(6-(pyrimidin-2-ylamino)pyridin-2-yl)morpholino)acetamide;
2′-(4-(2,4-dimethylpyrimidine-5-carbonyl)morpholin-2-yl)-N,6′-dimethyl-[3,4′-bipyridine]-6-carboxamide;
(2-(4-isonicotinoylmorpholin-2-yl)quinolin-4-yl)(morpholino)methanone;
pyridin-4-yl(2-(5-(3-(trifluoromethyl)benzyl)pyridin-2-yl)morpholino)methanone;
2-phenyl-1-(2-(5-(3-(trifluoromethyl)benzyl)pyridin-2-yl)morpholino)ethan-1-one;
(4-fluorophenyl)(2-(5-(3-(trifluoromethyl)benzyl)pyridin-2-yl)morpholino)methanone;
5-fluoro-N-(6-(4-((1-methyl-1H-indol-3-yl)methyl)morpholin-2-yl)pyridin-2-yl)pyridin-2-amine;
N-(6-(4-((1-ethyl-6-fluoro-1H-indol-3-yl)methyl)morpholin-2-yl)pyridin-2-yl)pyrazin-2-amine;
3-(1H-indazol-1-yl)-1-(2-(6-((4-methylthiazol-2-yl)amino)pyridin-2-yl)morpholino)propan-1-one;
N-(6-(4-((5-fluoro-1-methyl-1H-indol-3-yl)methyl)morpholin-2-yl)pyridin-2-yl)-4-methylthiazol-2-amine;
5-methyl-N-(2-methyl-6-(4-(quinolin-6-ylmethyl)morpholin-2-yl)pyridin-4-yl)thiazol-2-amine;
4-((1,5-dimethyl-1H-pyrazol-4-yl)methyl)-2-(6-(4-fluorobenzyl)pyridin-2-yl)morpholine;
1-(2-(6-benzylpyridin-2-yl)morpholino)-3-(1H-tetrazol-1-yl)propan-1-one;
4-((1,3-dimethyl-1H-pyrazol-4-yl)methyl)-2-(6-(2-fluorobenzyl)pyridin-2-yl)morpholine;
1-(2-(6-benzylpyridin-2-yl)morpholino)-3-(4-fluorophenyl)propan-1-one;
3-(3,5-dimethyl-1H-1,2,4-triazol-1-yl)-1-(2-(4-(4-fluorobenzyl)-6-methylpyridin-2-yl)morpholino)propan-1-one;
1-(2-(6-methyl-4-(3-(trifluoromethyl)benzyl)pyridin-2-yl)morpholino)-2-(1H-pyrazol-1-yl)ethan-1-one;
1-(2-(6-benzylpyridin-2-yl)morpholino)-2-(4-fluorophenyl)ethan-1-one;
2-(3,5-dimethyl-1H-pyrazol-1-yl)-1-(2-(6-methyl-4-(2-methylbenzyl)pyridin-2-yl)morpholino)ethan-1-one;
(6-aminopyridin-3-yl)(2-(4-(2-fluorobenzyl)-6-methylpyridin-2-yl)morpholino)methanone:
1-(2-(6-(3-chlorobenzyl)pyridin-2-yl)morpholino)-2-(4-fluorophenyl)ethan-1-one;
4-((1,5-dimethyl-1H-pyrazol-4-yl)methyl)-2-(6-(2-methylbenzyl)pyridin-2-yl)morpholine;
2-(4-benzyl-6-methylpyridin-2-yl)-4-((1,3-dimethyl-1H-pyrazol-4-yl)methyl)morpholine;
N,N-dimethyl-5-((2-(6-methyl-4-(4-methylbenzyl)pyridin-2-yl)morpholino)methyl)pyrimidin-2-amine;
N,N-dimethyl-5-((2-(6-methyl-4-(4-methylbenzyl)pyridin-2-yl)morpholino)methyl)pyrimidin-2-amine;
(1,2-dimethyl-1H-benzo[d]imidazol-5-yl)(2-(6-methyl-4-(pyridin-2-ylamino)pyridin-2-yl)morpholino)methanone;
3-(3,5-dimethyl-1H-1,2,4-triazol-1-yl)-1-(2-(6-(4-fluorobenzyl)pyridin-2-yl)morpholino)propan-1-one;
2-(4-(4-fluorobenzyl)-6-methylpyridin-2-yl)-4-((1-methyl-1H-pyrazol-4-yl)methyl)morpholine;
1-(2-(6-(2,6-difluorobenzyl)pyridin-2-yl)morpholino)-2-morpholinoethan-1-one;
(2-(6-(2,6-difluorobenzyl)pyridin-2-yl)morpholino)(2-(methylamino)pyridin-4-yl)methanone;
1-(2-(5-(3-chlorobenzyl)pyridin-2-yl)morpholino)-3-(3,5-dimethyl-TH-1,2,4-triazol-1-yl)propan-1-one;
3-((2-(6-(4-methoxyphenyl)pyridin-2-yl)morpholino)methyl)-N,N-dimethylpyridin-2-amine;
1-(2-(6-benzylpyridin-2-yl)morpholino)-3-phenylpropan-1-one:
(2-(dimethylamino)-4-methylpyrimidin-5-yl)(2-(6-methyl-4-(4-methylbenzyl)pyridin-2-yl)morpholino)methanone;
(2-(6-methyl-4-(3-(trifluoromethyl)benzyl)pyridin-2-yl)morpholino)(pyridin-3-yl)methanone;
(4-(ethoxymethyl)phenyl)(2-(6-methyl-4-((3-methylpyridin-2-yl)amino)pyridin-2-yl)morpholino)methanone;
5-((2-(6-(2-chlorobenzyl)pyridin-2-yl)morpholino)methyl)-N,N-dimethylpyrimidin-2-amine;
2-(1-ethylpiperidin-4-yl)-1-(2-(3′-methoxy-[1,1′-biphenyl]-4-yl)morpholino)ethan-1-one;
2-(1-ethylpiperidin-4-yl)-1-(3-(5-(3-methoxyphenyl)pyridin-2-yl)piperidin-1-yl)ethan-1-one;
2-(1-ethylpiperidin-4-yl)-1-(1-(5-(3-methoxyphenyl)pyridin-2-yl)piperidin-3-yl)ethan-1-one;
1-ethylpiperidin-4-yl 2-(5-(3-methoxyphenyl)pyridin-2-yl)morpholine-4-carboxylate;
4-(2-(1-ethylpiperidin-4-yl)ethyl)-2-(5-(3-methoxyphenyl)pyridin-2-yl)morpholine;
2-(1-ethylpiperidin-4-yl)-1-(2-(5-(4-fluorobenzyl)pyridin-2-yl)morpholino)ethan-1-one;
1-(2-(5-(4-fluorobenzyl)pyrazin-2-yl)morpholino)-3-(1H-indazol-1-yl)propan-1-one;
1-(2-(5-(4-fluorobenzyl)pyrimidin-2-yl)morpholino)-3-(1H-indazol-1-yl)propan-1-one;
3-(1H-indazol-1-yl)-1-(2-(5-(3-methoxyphenyl)pyridin-2-yl)morpholino)propan-1-one;
1-(2-(5-(4-fluorophenoxy)pyridin-2-yl)morpholino)-3-(1H-indazol-1-yl)propan-1-one;
2-(1-ethylpiperidin-4-yl)-1-(2-(6-(3-methoxyphenyl)pyridin-3-yl)morpholino)ethan-1-one;
2-(3-ethyl-3-azabicyclo[3.1.0]hexan-6-yl)-1-(2-(5-(3-methoxyphenyl)pyridin-2-yl)morpholino)ethan-1-one;
1-(2-(4-(4-fluorobenzyl)phenyl)morpholino)-3-(1H-pyrazol-1-yl)propan-1-one;
4-(3-(1H-indazol-1-yl)propyl)-2-(4-(4-fluorobenzyl)phenyl)morpholine;
2-(1-ethylpiperidin-4-yl)-1-(2-(5-phenylpyridin-2-yl)morpholino)ethan-1-one; and
2-(4-ethylpiperazin-1-yl)-1-(2-(5-(3-methoxyphenyl)pyridin-2-yl)morpholino)ethan-1-one.

Embodiment 25 provides a method of treating, preventing, and/or ameliorating an airway disease in a subject, the method comprising administering to the subject a compound selected from the group consisting of
2-(4-(2-(3,4-dimethylphenyl)acetyl)morpholin-2-yl)-N-(2-hydroxyethyl)quinoline-4-carboxamide;
N-(2-hydroxyethyl)-2-(4-((1-methyl-1H-indol-3-yl)methyl)morpholin-2-yl)quinoline-4-carboxamide;
N-(2-methoxyethyl)-2-(4-phenethylmorpholin-2-yl)quinoline-4-carboxamide;
2-(4-((1H-indol-5-yl)methyl)morpholin-2-yl)-N-(2-hydroxyethyl)-N-methylquinoline-4-carboxamide;
2-(4-benzoylmorpholin-2-yl)-N-isobutylquinoline-4-carboxamide;
N-isopropyl-2-(4-(quinolin-4-ylmethyl)morpholin-2-yl)quinoline-4-carboxamide;
N-methyl-2-(4-((3-phenyl-1H-pyrazol-4-yl)methyl)morpholin-2-yl)quinoline-4-carboxamide;
1-(1′-(2-(2-fluorophenyl)acetyl)-[2,4′-bipiperidin]-1-yl)-3-(1H-indazol-1-yl)propan-1-one;
2-(4-(2-(4-fluorophenyl)acetyl)morpholin-2-yl)-N-isobutylquinoline-4-carboxamide;
2-(4-((1H-indol-5-yl)methyl)morpholin-2-yl)-N-(2-methoxyethyl)quinoline-4-carboxamide;
N-(2-hydroxyethyl)-2-(4-(quinolin-6-ylmethyl)morpholin-2-yl)quinoline-4-carboxamide
N-isobutyl-2-(4-(2-phenylacetyl)morpholin-2-yl)quinoline-4-carboxamide;
3-(6-(4-(4,6-dimethylpyrimidin-2-yl)morpholin-2-yl)pyridin-3-yl)propenamide;
N,N-dimethyl-2-(2-(pyridin-2-yl)morpholino)acetamide;
3-(1H-indazol-1-yl)-1-(2-(5-(4-methoxybenzyl)pyridin-2-yl)pyrrolidin-1-yl)propan-1-one;
1-(2-(6-(3-fluorobenzyl)pyrazin-2-yl)pyrrolidin-1-yl)-3-(1H-indazol-1-yl)propan-1-one;
4-(3-(1H-indazol-1-yl)propanoyl)-3-(3-(pyridin-4-yl)benzyl)piperazin-2-one;
(2-(6-(dimethylamino)pyridin-2-yl)morpholino)(5-methylpyridin-3-yl)methanone;
1-(2-(pyrazin-2-yl)morpholino)-2-(pyridin-3-yl)ethan-1-one; and
N-(2-hydroxyethyl)-N-methyl-2-(morpholin-2-yl)quinoline-4-carboxamide;
   or a salt, solvate, prodrug, stereoisomer, isotopologue, or tautomer thereof, or any mixtures thereof

Embodiment 26 provides the method of Embodiment 25, wherein contraction of an airway smooth muscle cell is inhibited.

Embodiment 27 provides the method of Embodiment 25 or 26, wherein dilation of an airway smooth muscle cell is promoted.

Embodiment 28 provides the method of any one of Embodiments 25-27, wherein the airway disease is at least one selected from the group consisting of asthma, chronic obstructive pulmonary disease (COPD), chronic bronchitis, bronchiectasis, and cystic fibrosis.

Embodiment 29 provides a method of inhibiting bronchoconstriction and/or promoting bronchodilation in a subject, the method comprising administering to the subject a compound selected from the group consisting of.
2-(4-(2-(3,4-dimethylphenyl)acetyl)morpholin-2-yl)-N-(2-hydroxyethyl)quinoline-4-carboxamide;
N-(2-hydroxyethyl)-2-(4-((1-methyl-1H-indol-3-yl)methyl)morpholin-2-yl)quinoline-4-carboxamide;
N-(2-methoxyethyl)-2-(4-phenethylmorpholin-2-yl)quinoline-4-carboxamide;
2-(4-((1H-indol-5-yl)methyl)morpholin-2-yl)-N-(2-hydroxyethyl)-N-methylquinoline-4-carboxamide;
2-(4-benzoylmorpholin-2-yl)-N-isobutylquinoline-4-carboxamide;
N-isopropyl-2-(4-(quinolin-4-ylmethyl)morpholin-2-yl)quinoline-4-carboxamide;
N-methyl-2-(4-((3-phenyl-1H-pyrazol-4-yl)methyl)morpholin-2-yl)quinoline-4-carboxamide;
1-(1′-(2-(2-fluorophenyl)acetyl)-[2,4′-bipiperidin]-1-yl)-3-(1H-indazol-1-yl)propan-1-one;
2-(4-(2-(4-fluorophenyl)acetyl)morpholin-2-yl)-N-isobutylquinoline-4-carboxamide;
2-(4-((1H-indol-5-yl)methyl)morpholin-2-yl)-N-(2-methoxyethyl)quinoline-4-carboxamide;
N-(2-hydroxyethyl)-2-(4-(quinolin-6-ylmethyl)morpholin-2-yl)quinoline-4-carboxamide
N-isobutyl-2-(4-(2-phenylacetyl)morpholin-2-yl)quinoline-4-carboxamide;
3-(6-(4-(4,6-dimethylpyrimidin-2-yl)morpholin-2-yl)pyridin-3-yl)propenamide;
N,N-dimethyl-2-(2-(pyridin-2-yl)morpholino)acetamide;
3-(1H-indazol-1-yl)-1-(2-(5-(4-methoxybenzyl)pyridin-2-yl)pyrrolidin-1-yl)propan-1-one;
1-(2-(6-(3-fluorobenzyl)pyrazin-2-yl)pyrrolidin-1-yl)-3-(1H-indazol-1-yl)propan-1-one;
4-(3-(1H-indazol-1-yl)propanoyl)-3-(3-(pyridin-4-yl)benzyl)piperazin-2-one;
(2-(6-(dimethylamino)pyridin-2-yl)morpholino)(5-methylpyridin-3-yl)methanone;
1-(2-(pyrazin-2-yl)morpholino)-2-(pyridin-3-yl)ethan-1-one; and
N-(2-hydroxyethyl)-N-methyl-2-(morpholin-2-yl)quinoline-4-carboxamide;
   or a salt, solvate, prodrug, stereoisomer, isotopologue, or tautomer thereof, or any mixtures thereof.

Embodiment 30 provides the method of any one of Embodiments 25-29, wherein Gα₁₂ is inhibited.

Embodiment 31 provides the method of any one of Embodiments 25-30, further comprising administering to the subject a therapeutically effective amount of one or more additional agents.

Embodiment 32 provides the method of Embodiment 31, wherein the one or more additional agents are selected from the group consisting of a phosphoinositide 3-kinase (PI3K) inhibitor and a rho kinase (ROCK) inhibitor.

Embodiment 33 provides the method of Embodiment 32, wherein the PI3K inhibitor is selected from the group consisting of idelalisib, copanlisib, duvelisib, alpelisib, umbralisib, buparlisib, dactolisib, leniolisib, parsaclisib, paxalisib, taselisib, zandelisib, inavolisib, apitolisib, bimarlisib, eganelisib, fimepinostat, gedatolisib, linperlisib, nemiralisib, pictilisib, pilaralisib, samotolisib, seletalisib, serabelisib, sonolisib, tenalisib, voxtalisib, AMG 319, AZD8186, GSK2636771, SF1126, acalisib, omipalisib, AZD8835, CAL263, GSK1059615, MEN1611, PWT33597, TG100-115, and ZSTK474.

Embodiment 34 provides the method of Embodiment 32, wherein the ROCK inhibitor is selected from the group consisting of rhosin, AT-13148, BA-210, β-elemene, chroman 1, DJ4, fasudil, GSK-576371, GSK429286A, H-1152, hydroxyfasudil, ibuprofen, LX-7101, netarsudil, RKI-1447, ripasudil, TCS-7001, thiazovivin, verosudil, Y-27632, Y-30141, Y-33075, and Y-39983.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A method of treating, preventing, and/or ameliorating an airway disease in a subject, the method comprising administering to the subject at least one selected from the group consisting of:
(a) a compound of formula (I), or a salt, solvate, prodrug, stereoisomer, isotopologue, or tautomer thereof, or any mixtures thereof: 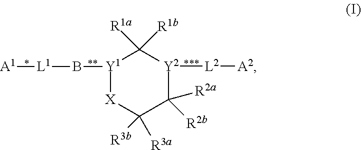
wherein:
A¹ is selected from the group consisting of optionally substituted phenyl and optionally substituted C₁-C₁₀ heterocyclyl;
A² is selected from the group consisting of optionally substituted phenyl, optionally substituted C₁-C₁₀ heterocyclyl, and NH(optionally substituted C₂-C₈ heterocyclyl);
B is selected from the group consisting of 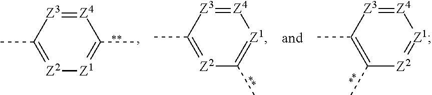
L¹ is selected from the group consisting of a bond, —NR⁴—, —O—, and optionally substituted C₁-C₃ alkylenyl;
L² is selected from the group consisting of a bond, —C(═O)—, —C(═O)O—, —C(═O)(optionally substituted C₁-C₃ alkylenyl)-, and optionally substituted C₁-C₃ alkylenyl;
X is selected from the group consisting of —O— and —C(R^{5a})(R^{5b})—,
Y¹ and Y² are each independently selected from the group consisting of N and CR⁶,
wherein Y¹ and Y² cannot be both N, and
wherein if X is O, then Y¹ is CR⁶;
Z¹ is selected from the group consisting of CR^{7a} and N, Z² is selected from the group consisting of CR^{7b} and N, Z³ is selected from the group consisting of CR^{7c} and N, and Z⁴ is selected from the group consisting of CR^{7d} and N,
wherein 0-2 selected from the group consisting of Z¹, Z², Z³, and Z⁴ are N;
R^{1a}, R^{1b}, R^{2a}, R^{2b}, R^{3a}, and R^{3b} are each independently selected from the group consisting of H, optionally substituted C₁-C₆ alkyl, optionally substituted C₃-C₈ cycloalkyl, optionally substituted C₁-C₆ alkoxy, and halogen;
R⁴ is selected from the group consisting of H and optionally substituted C₁-C₆ alkyl;
R^{5a} and R^{5b} are each independently selected from the group consisting of H, optionally substituted C₁-C₆ alkyl, optionally substituted C₃-C₈ cycloalkyl, optionally substituted C₁-C₆ alkoxy, and halogen;
R⁶ is selected from the group consisting of H, optionally substituted C₁-C₆ alkyl, optionally substituted C₃-C₈ cycloalkyl, optionally substituted C₁-C₆ alkoxy, and halogen;
R^{7a}, R^{7b}, R^{7c}, and R^{7d} are each independently selected from the group consisting of H, optionally substituted C₁-C₆ alkyl, optionally substituted C₃-C₈ cycloalkyl, optionally substituted C₁-C₆ alkoxy, optionally substituted phenyl, optionally substituted C₂-C₈ heterocyclyl, halogen, CN, NO₂, OR^{a}, N(R^{a})(R^{b}), C(═O)R^{a}, C(═O)OR^{a}, OC(═O)R^{a}, C(═O)N(R^{a})(R^{b}), S(═O)₂N(R^{a})(R^{b}), NR^{a}C(═O)R^{b}, and NR^{a}S(═O)₂R^{b},
wherein two vicinal substituents selected from the group consisting of R^{7a}, R^{7b}, R^{7c}, and R^{7d} can combine with the atoms to which they are bound to form an optionally substituted phenyl or optionally substituted C₂-C₈ heterocyclyl;
R^{a} and R^{b} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl;
* indicates a bond between A¹ and L¹;
** indicates a bond between B and Y¹; and
*** indicates a bond between Y² and L²;
(b) a compound selected from the group consisting of:
2-(4-(2-(3,4-dimethylphenyl)acetyl)morpholin-2-yl)-N-(2-hydroxyethyl)quinoline-4-carboxamide;
N-(2-hydroxyethyl)-2-(4-((1-methyl-1H-indol-3-yl)methyl)morpholin-2-yl)quinoline-4-carboxamide;
N-(2-methoxyethyl)-2-(4-phenethylmorpholin-2-yl)quinoline-4-carboxamide;
2-(4-((1H-indol-5-yl)methyl)morpholin-2-yl)-N-(2-hydroxyethyl)-N-methylquinoline-4-carboxamide;
2-(4-benzoylmorpholin-2-yl)-N-isobutylquinoline-4-carboxamide;
N-isopropyl-2-(4-(quinolin-4-ylmethyl)morpholin-2-yl)quinoline-4-carboxamide;
N-methyl-2-(4-((3-phenyl-1H-pyrazol-4-yl)methyl)morpholin-2-yl)quinoline-4-carboxamide;
1-(1′-(2-(2-fluorophenyl)acetyl)-[2,4′-bipiperidin]-1-yl)-3-(1H-indazol-1-yl)propan-1-one;
2-(4-(2-(4-fluorophenyl)acetyl)morpholin-2-yl)-N-isobutylquinoline-4-carboxamide;
2-(4-((1H-indol-5-yl)methyl)morpholin-2-yl)-N-(2-methoxyethyl)quinoline-4-carboxamide;
N-(2-hydroxyethyl)-2-(4-(quinolin-6-ylmethyl)morpholin-2-yl)quinoline-4-carboxamide
N-isobutyl-2-(4-(2-phenylacetyl)morpholin-2-yl)quinoline-4-carboxamide;
3-(6-(4-(4,6-dimethylpyrimidin-2-yl)morpholin-2-yl)pyridin-3-yl)propenamide;
N,N-dimethyl-2-(2-(pyridin-2-yl)morpholino)acetamide;
3-(1H-indazol-1-yl)-1-(2-(5-(4-methoxybenzyl)pyridin-2-yl)pyrrolidin-1-yl)propan-1-one;
1-(2-(6-(3-fluorobenzyl)pyrazin-2-yl)pyrrolidin-1-yl)-3-(1H-indazol-1-yl)propan-1-one;
4-(3-(1H-indazol-1-yl)propanoyl)-3-(3-(pyridin-4-yl)benzyl)piperazin-2-one;
(2-(6-(dimethylamino)pyridin-2-yl)morpholino)(5-methylpyridin-3-yl)methanone;
1-(2-(pyrazin-2-yl)morpholino)-2-(pyridin-3-yl)ethan-1-one; and
N-(2-hydroxyethyl)-N-methyl-2-(morpholin-2-yl)quinoline-4-carboxamide;
or a salt, solvate, prodrug, stereoisomer, isotopologue, or tautomer thereof, or any mixtures thereof.

2. The method of claim 1, wherein at least one of the following applies:
(a) contraction of an airway smooth muscle cell is inhibited; and
(b) dilation of an airway smooth muscle cell is promoted.

3. (canceled)

4. The method of claim 1, wherein the airway disease is at least one selected from the group consisting of asthma, chronic obstructive pulmonary disease (COPD), chronic bronchitis, bronchiectasis, and cystic fibrosis.

5. A method of inhibiting bronchoconstriction or promoting bronchodilation in a subject, the method comprising administering to the subject at least one of the following:
(a) a compound of formula (I), or a salt, solvate, prodrug, stereoisomer, isotopologue, or tautomer thereof, or any mixtures thereof: 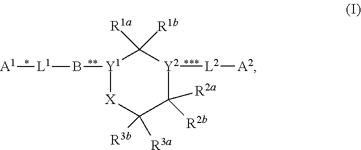
wherein:
A¹ is selected from the group consisting of optionally substituted phenyl and optionally substituted C₁-C₁₀ heterocyclyl;
A² is selected from the group consisting of optionally substituted phenyl, optionally substituted C₁-C₁₀ heterocyclyl, and NH(optionally substituted C₂-C₈ heterocylyl);
B is selected from the group consisting of 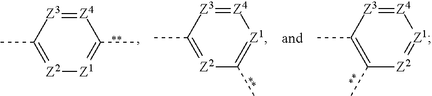
L¹ is selected from the group consisting of a bond, —NR⁴—, —O—, and optionally substituted C₁-C₃ alkylenyl;
L² is selected from the group consisting of a bond, —C(═O)—, —C(═O)O—, —C(═O)(optionally substituted C₁-C₃ alkylenyl)-, and optionally substituted C₁-C₃ alkylenyl;
X is selected from the group consisting of —O— and —C(R^{5a})(R^{5b})—,
Y¹ and Y² are each independently selected from the group consisting of N and CR⁶,
wherein no more than one of Y¹ and Y² is N, and
wherein if X is O, then Y¹ is CR⁶;
Z¹ is selected from the group consisting of CR^{7a} and N, Z² is selected from the group consisting of CR^{7b} and N, Z³ is selected from the group consisting of CR^{7c} and N, and Z⁴ is selected from the group consisting of CR^{7d} and N,
wherein 0-2 selected from the group consisting of Z¹, Z², Z³, and Z⁴ are N;
R^{1a}, R^{1b}, R^{2a}, R^{2b}, R^{3a}, and R^{3b} are each independently selected from the group consisting of H, optionally substituted C₁-C₆ alkyl, optionally substituted C₃-C₈ cycloalkyl, optionally substituted C₁-C₆ alkoxy, and halogen;
R⁴ is selected from the group consisting of H and optionally substituted C₁-C₆ alkyl;
R^{5a} and R^{5b} are each independently selected from the group consisting of H, optionally substituted C₁-C₆ alkyl, optionally substituted C₃-C₈ cycloalkyl, optionally substituted C₁-C₆ alkoxy, and halogen;
R⁶ is selected from the group consisting of H, optionally substituted C₁-C₆ alkyl, optionally substituted C₃-C₈ cycloalkyl, optionally substituted C₁-C₆ alkoxy, and halogen;
R^{7a}, R^{7b}, R^{7c}, and R^{7d} are each independently selected from the group consisting of H, optionally substituted C₁-C₆ alkyl, optionally substituted C₃-C₈ cycloalkyl, optionally substituted C₁-C₆ alkoxy, optionally substituted phenyl, optionally substituted C₂-C₈ heterocyclyl, halogen, CN, NO₂, OR^{a}, N(R^{a})(R^{b}), C(═O)R^{a}, C(═O)OR^{a}, OC(═O)R^{a}, C(═O)N(R^{a})(R^{b}), S(═O)₂N(R^{a})(R), NR^{a}C(═O)R^{b}, and NR^{a}S(═O)₂R^{b};
wherein two vicinal substituents selected from the group consisting of R^{7a}, R^{7b}, R^{7c}, and R^{7d} can combine with the atoms to which they are bound to form an optionally substituted phenyl or optionally substituted C₂-C₈ heterocyclyl;
R^{a} and R^{b} are each independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl;
* indicates a bond between A¹ and L¹;
** indicates a bond between B and Y¹; and
*** indicates a bond between Y² and L²; and
(b) a compound selected from the group consisting of:
2-(4-(2-(3,4-dimethylphenyl)acetyl)morpholin-2-yl)-N-(2-hydroxyethyl)quinoline-4-carboxamide;
N-(2-hydroxyethyl)-2-(4-((1-methyl-1H-indol-3-yl)methyl)morpholin-2-yl)quinoline-4-carboxamide;
N-(2-methoxyethyl)-2-(4-phenethylmorpholin-2-yl)quinoline-4-carboxamide;
2-(4-((1H-indol-5-yl)methyl)morpholin-2-yl)-N-(2-hydroxyethyl)-N-methylquinoline-4-carboxamide;
2-(4-benzoylmorpholin-2-yl)-N-isobutylquinoline-4-carboxamide;
N-isopropyl-2-(4-(quinolin-4-ylmethyl)morpholin-2-yl)quinoline-4-carboxamide;
N-methyl-2-(4-((3-phenyl-1H-pyrazol-4-yl)methyl)morpholin-2-yl)quinoline-4-carboxamide;
1-(1′-(2-(2-fluorophenyl)acetyl)-[2,4′-bipiperidin]-1-yl)-3-(1H-indazol-1-yl)propan-1-one;
2-(4-(2-(4-fluorophenyl)acetyl)morpholin-2-yl)-N-isobutylquinoline-4-carboxamide;
2-(4-((1H-indol-5-yl)methyl)morpholin-2-yl)-N-(2-methoxyethyl)quinoline-4-carboxamide;
N-(2-hydroxyethyl)-2-(4-(quinolin-6-ylmethyl)morpholin-2-yl)quinoline-4-carboxamide
N-isobutyl-2-(4-(2-phenylacetyl)morpholin-2-yl)quinoline-4-carboxamide;
3-(6-(4-(4,6-dimethylpyrimidin-2-yl)morpholin-2-yl)pyridin-3-yl)propenamide;
N,N-dimethyl-2-(2-(pyridin-2-yl)morpholino)acetamide;
3-(1H-indazol-1-yl)-1-(2-(5-(4-methoxybenzyl)pyridin-2-yl)pyrrolidin-1-yl)propan-1-one;
1-(2-(6-(3-fluorobenzyl)pyrazin-2-yl)pyrrolidin-1-yl)-3-(1H-indazol-1-yl)propan-1-one;
4-(3-(1H-indazol-1-yl)propanoyl)-3-(3-(pyridin-4-yl)benzyl)piperazin-2-one;
(2-(6-(dimethylamino)pyridin-2-yl)morpholino)(5-methylpyridin-3-yl)methanone;
1-(2-(pyrazin-2-yl)morpholino)-2-(pyridin-3-yl)ethan-1-one; and
N-(2-hydroxyethyl)-N-methyl-2-(morpholin-2-yl)quinoline-4-carboxamide;
or a salt, solvate, prodrug, stereoisomer, isotopologue, or tautomer thereof, or any mixtures thereof.

6. The method of claim 1, wherein Gα₁₂ is inhibited.

7. The method of claim 1, further comprising administering to the subject a therapeutically effective amount of one or more additional agents, optionally wherein the one or more additional agents are selected from the group consisting of a phosphoinositide 3-kinase (PI3K) inhibitor and a rho kinase (ROCK) inhibitor.

8. (canceled)

9. The method of claim 7, wherein at least one of the following applies:
(a) the PI3K inhibitor is at least one selected from the group consisting of idelalisib, copanlisib, duvelisib, alpelisib, umbralisib, buparlisib, dactolisib, leniolisib, parsaclisib, paxalisib, taselisib, zandelisib, inavolisib, apitolisib, bimarlisib, eganelisib, fimepinostat, gedatolisib, linperlisib, nemiralisib, pictilisib, pilaralisib, samotolisib, seletalisib, serabelisib, sonolisib, tenalisib, voxtalisib, AMG 319, AZD8186, GSK2636771, SF1126, acalisib, omipalisib, AZD8835, CAL263, GSK1059615, MEN1611, PWT33597, TG100-115, and ZSTK474; and
(b) the ROCK inhibitor is at least one selected from the group consisting of rhosin, AT-13148, BA-210, β-elemene, chroman 1, DJ4, fasudil, GSK-576371, GSK429286A, H-1152, hydroxyfasudil, ibuprofen, LX-7101, netarsudil, RKI-1447, ripasudil, TCS-7001, thiazovivin, verosudil, Y-27632, Y-30141, Y-33075, and Y-39983.

10. (canceled)

11. The method of claim 1, wherein the compound of formula (I) is selected from the group consisting of:

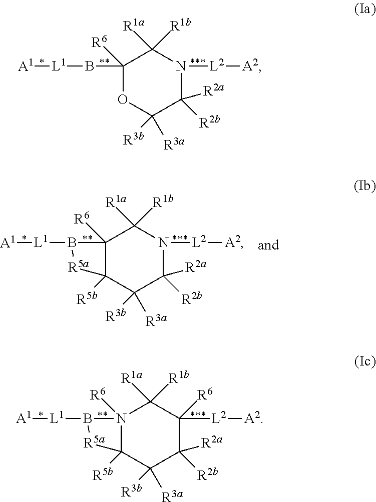

12. The method of claim 11, wherein the compound of formula (I) is selected from the group consisting of:

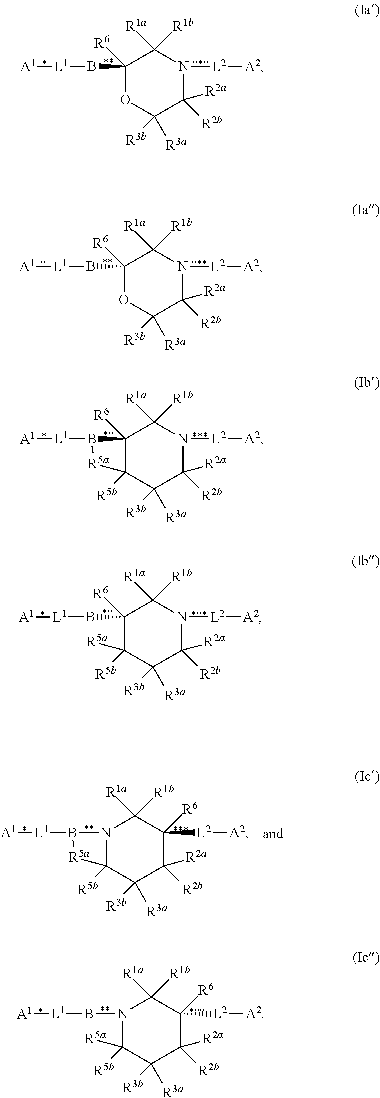

13. The method of claim 1, wherein one of the following applies:
(a) R^{1a}, R^{1b}, R^{2a}, R^{2b}, R^{3a}, and R^{3b} are each independently H;
(b) R^{1a}, R^{1b}, R^{2a}, R^{2b}, R^{3a}, R^{3b}, R^{5a}, and R^{5b} are each independently H;
(c) R^{1a}, R^{1b}, R^{2a}, R^{2b}, R^{3a}, R^{3b}, and R⁶ are each independently H; or
(d) R^{1a}, R^{1b}, R^{2a}, R^{2b}, R^{3a}, R^{3b}, R^{5a}, R^{5b}, and R⁶ are each independently H.

14. The method of claim 1, wherein B is selected from the group consisting of:

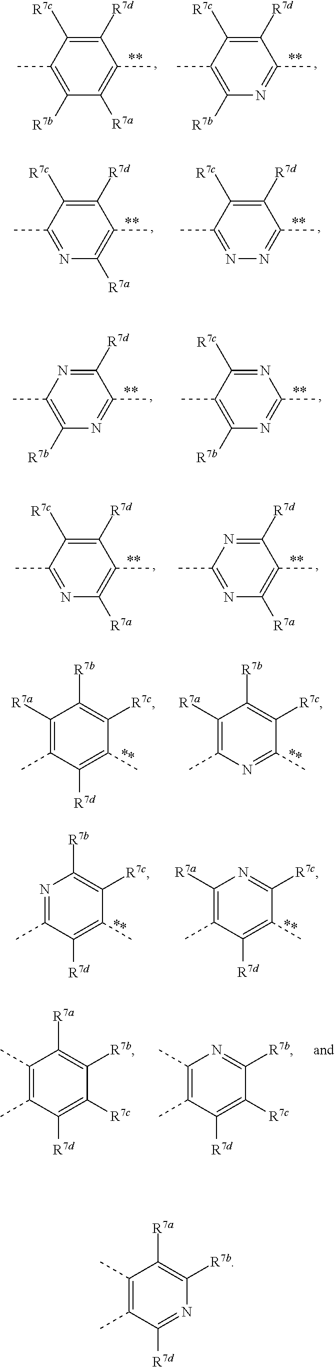

15. The method of claim 1, wherein at least one of the following applies:
(a) at least one selected from the group consisting of R^{7a}, R^{7b}, R^{7c}, and R^{7d} is H;
(b) at least two selected from the group consisting of R^{7a}, R^{7b}, R^{7c}, and R^{7d} are H;
(c) at least three selected from the group consisting of R^{7a}, R^{7b}, R^{7c}, and R^{7d} are H;
(d) each of R^{7a}, R^{7b}, R^{7c}, and R^{7d} are H; and
(e) two vicinal substituents selected from the group consisting of R^{7a}, R^{7b}, R^{7c}, and R^{7d} combine to form phenyl.

16. The method of claim 1, wherein B is selected from the group consisting of

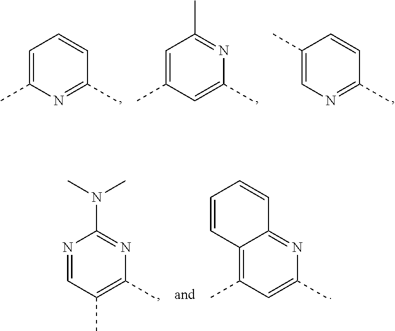

17. The method of claim 1, wherein at least one of the following applies:
(a) the phenyl or heterocyclyl in A¹ is optionally substituted with at least one substituent selected from the group consisting of methyl, trifluoromethyl, fluorine, chlorine, methoxy, and C(═O)NH₂; and
(b) A¹ is selected from the group consisting of phenyl,

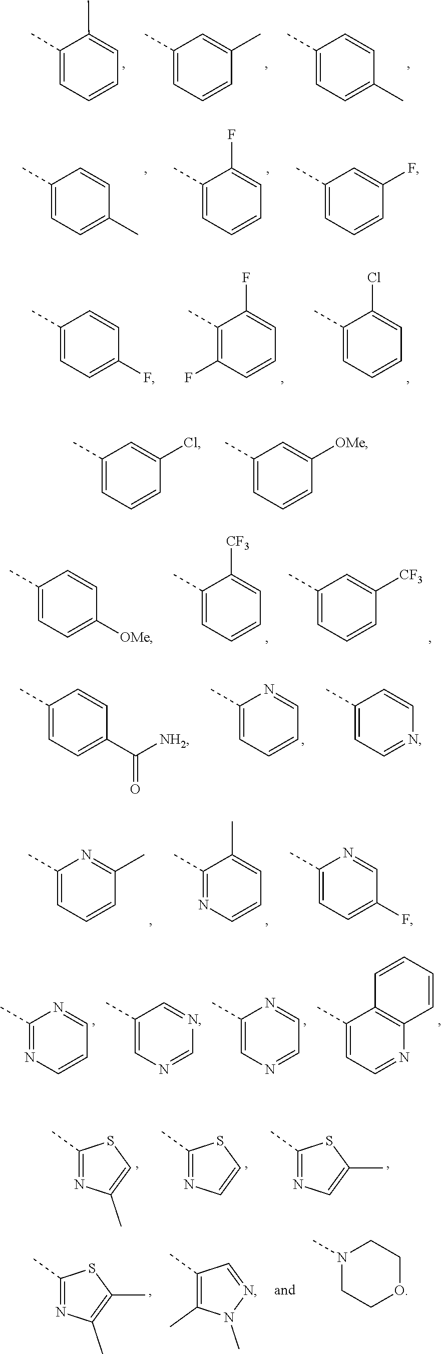

18. (canceled)

19. The method of claim 1, wherein A² is selected from the group consisting of: 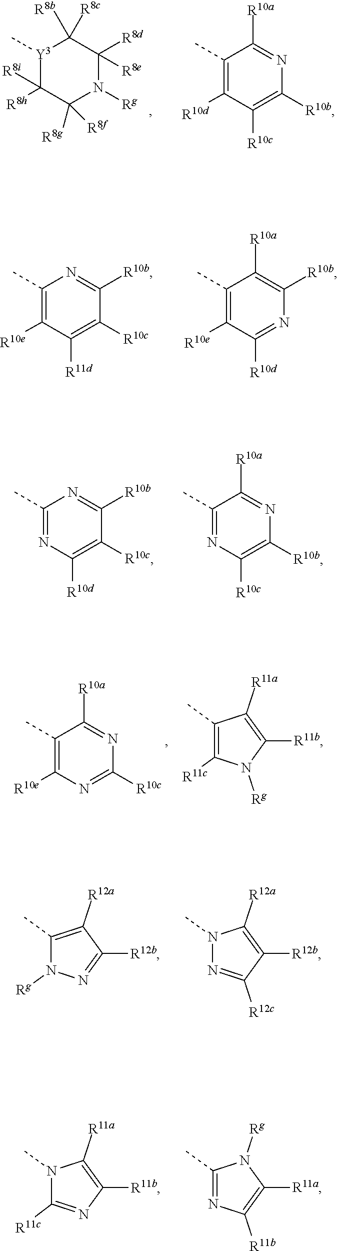 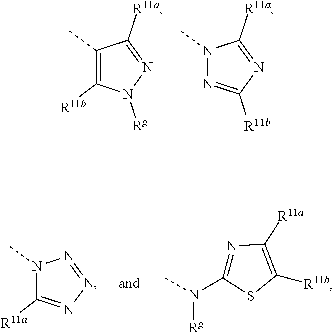
wherein:
R^{8a}, R^{8b}, R^{8c}, R^{8d}, R^{8e}, R^{8f}, R^{8g}, R^{8h}, and R⁸ⁱ are each independently selected from the group consisting of H, optionally substituted C₁-C₆ alkyl, optionally substituted C₃-C₈ cycloalkyl, and halogen,
wherein any two substituents selected from the group consisting of R^{8a}, R^{8b}, R^{8c}, R^{8d}, R^{8e}, R^{8f}, R^{8g}, R^{8h}, and R⁸ⁱ may combine with the atoms to which they are bound to form an optionally substituted C₃-C₈ cycloalkyl or optionally substituted C₂-C₈ heterocyclyl;
R⁹ is selected from the group consisting of H, optionally substituted C₁-C₆ alkyl, optionally substituted C₁-C₆ cycloalkyl, and optionally substituted benzyl;
R^{10a}, R^{10b}, R^{10c}, R^{10d}, and R^{10e}, if present, are each independently selected from the group consisting of H, optionally substituted C₁-C₆ alkyl, optionally substituted C₃-C₈ cycloalkyl, halogen, N(R^{a})(R^{b}), CN, and NO₂,
wherein no more than one of R^{10a}, R^{10b}, R^{10c}, R^{10d}, and R^{10e} is N(R^{a})(R^{b}), and two vicinal substituents selected from the group consisting of R^{10a}, R^{10b}, R^{10c}, R^{10d}, and R^{10e} can combine with the atoms to which they are bound to form an optionally substituted phenyl or optionally substituted C₂-C₈ heterocyclyl;
R^{11a}, R^{11b}, and R^{11c}, if present, are each independently selected from the group consisting of H, optionally substituted C₁-C₆ alkyl, optionally substituted C₃-C₈ cycloalkyl, halogen, CN, and NO₂,
wherein two vicinal substituents selected from the group consisting of R^{11a}, R^{11b}, and R^{11c} can combine with the atoms to which they are bound to form an optionally substituted phenyl or optionally substituted C₂-C₈ heterocyclyl; and
Y³ is selected from the group consisting of R^{8a} and N.

20. The method of claim 1, wherein A² is selected from the group consisting of:

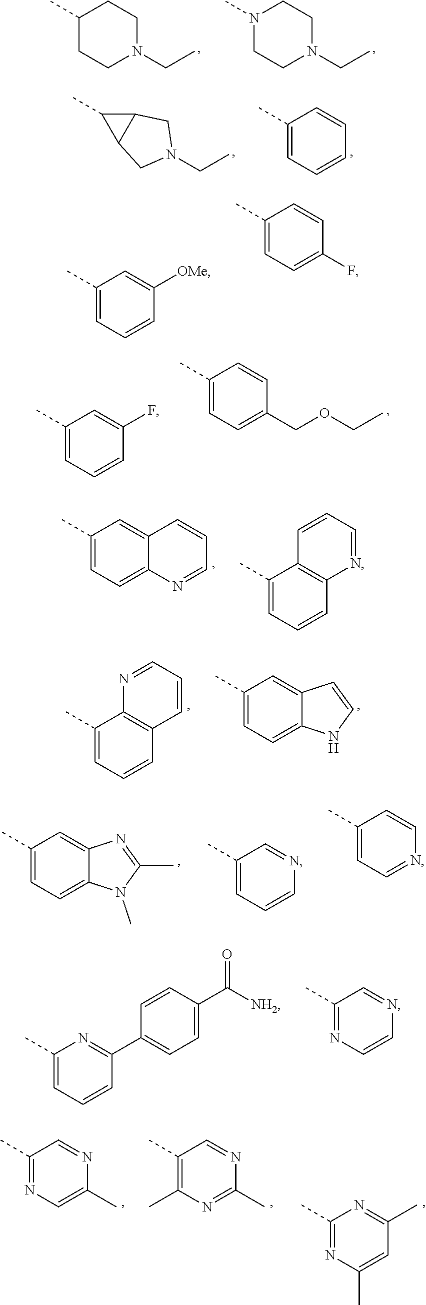

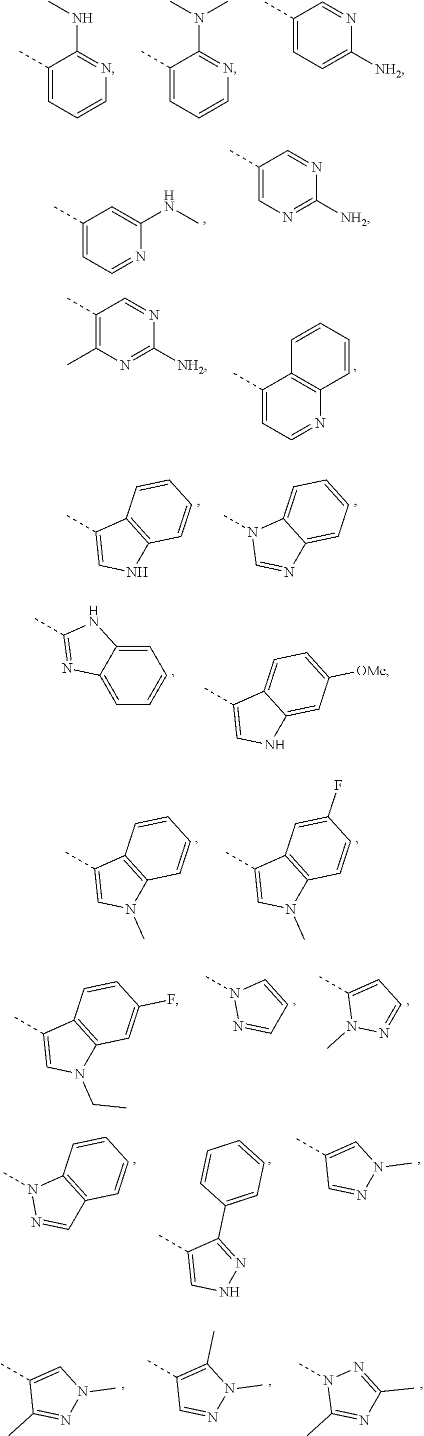

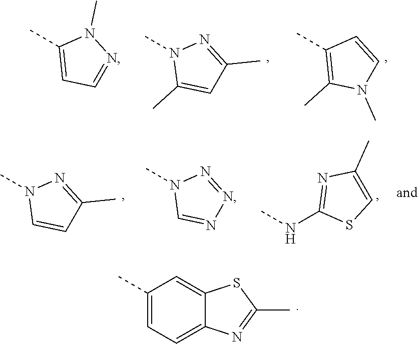

21. The method of claim 1, wherein L¹ is selected from the group consisting of a bond, —NH—, —O—, and —CH₂—.

22. The method of claim 1, wherein L² is selected from the group consisting of a bond, —CH₂—, —CH₂CH₂—, —CH₂CH₂CH₂—, —C(═O)—,

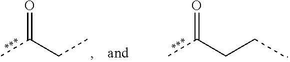

23. The method of claim 1, wherein each occurrence of optionally substituted alkyl, optionally substituted cycloalkyl, optionally substituted heterocyclyl, optionally substituted alkylenyl, optionally substituted alkoxy, optionally substituted benzyl, and optionally substituted phenyl, if present, is optionally substituted with at least one substituent selected from the group consisting of C₁-C₆ alkyl, C₁-C₆ heteroalkyl, C₃-C₈ cycloalkyl, C₁-C₆ haloalkyl, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, wherein each occurrence of R′ and R″ is independently selected from the group consisting of H, C₁-C₆ alkyl, C₃-C₈ cycloalkyl, C₁-C₆ haloalkyl, benzyl, and phenyl.

24. The method of claim 1, wherein the compound of formula (I) is selected from the group consisting of:
1-(2-(5-(4-fluorobenzyl)pyridin-2-yl)morpholino)-3-(1H-indazol-1-yl)propan-1-one;
2-(1-ethylpiperidin-4-yl)-1-(2-(5-(3-methoxyphenyl)pyridin-2-yl)morpholino)ethan-1-one;
(2-(5-(4-methylbenzyl)pyridin-2-yl)morpholino)(5-methylpyrazin-2-yl)methanone;
N-methyl-3-((2-(5-(3-(trifluoromethyl)benzyl)pyridin-2-yl)morpholino)methyl)pyridin-2-amine;
4-(pyridin-3-ylmethyl)-2-(5-(3-(trifluoromethyl)benzyl)pyridin-2-yl)morpholine;
2-(6-(4-fluorobenzyl)pyridin-2-yl)-4-(quinolin-6-ylmethyl)morpholine;
4-(6-(4-(quinolin-4-ylmethyl)morpholin-2-yl)pyridin-2-yl)benzamide;
morpholino(2-(4-phenethylmorpholin-2-yl)quinolin-4-yl)methanone;
(2-(4-(3-methoxybenzyl)morpholin-2-yl)quinolin-4-yl)(morpholino)methanone;
2-(4-(2-(3,4-dimethylphenyl)acetyl)morpholin-2-yl)-N-(2-hydroxyethyl)quinoline-4-carboxamide;
4-(6-(4-(4-(4-fluorophenyl)butanoyl)morpholin-2-yl)pyridin-3-yl)benzamide;
3-(1H-indol-3-yl)-1-(2-(6-(4-methoxyphenyl)pyridin-2-yl)morpholino)propan-1-one;
N-(2-hydroxyethyl)-2-(4-((1-methyl-1H-indol-3-yl)methyl)morpholin-2-yl)quinoline-4-carboxamide;
N-(2-methoxyethyl)-2-(4-phenethylmorpholin-2-yl)quinoline-4-carboxamide;
3-(1H-benzo[d]imidazol-2-yl)-1-(2-(6-(2-fluorobenzyl)pyridin-2-yl)morpholino)propan-1-one;
3-(1H-benzo[d]imidazol-2-yl)-1-(2-(5-(2-fluorobenzyl)pyridin-2-yl)morpholino)propan-1-one;
(2-(6-(2-chlorobenzyl)pyridin-2-yl)morpholino)(quinolin-5-yl)methanone;
(2-(6-benzylpyridin-2-yl)morpholino)(quinolin-8-yl)methanone;
2-(5-(4-methoxybenzyl)pyridin-2-yl)-4-(quinolin-5-ylmethyl)morpholine;
2-(4-benzylmorpholin-2-yl)-N-(thiazol-2-yl)quinoline-4-carboxamide;
(2-(5-(2-chlorobenzyl)pyridin-2-yl)morpholino)(quinolin-8-yl)methanone;
2-(4-((1H-indol-5-yl)methyl)morpholin-2-yl)-N-(2-hydroxyethyl)-N-methylquinoline-4-carboxamide;
2-(4-benzoylmorpholin-2-yl)-N-isobutylquinoline-4-carboxamide;
5-(4-fluorophenyl)-N,N-dimethyl-4-(4-(quinolin-4-ylmethyl)morpholin-2-yl)pyrimidin-2-amine;
1-(2-(2-(dimethylamino)-5-(3-fluorophenyl)pyrimidin-4-yl)morpholino)-2-(4-fluorophenyl)ethan-1-one;
N-isopropyl-2-(4-(quinolin-4-ylmethyl)morpholin-2-yl)quinoline-4-carboxamide;
N-methyl-2-(4-((3-phenyl-1H-pyrazol-4-yl)methyl)morpholin-2-yl)quinoline-4-carboxamide;
1-(2-(6-(2,6-difluorophenyl)pyridin-2-yl)morpholino)-3-(1H-indol-3-yl)propan-1-one;
(2-(4-benzylmorpholin-2-yl)quinolin-4-yl)(morpholino)methanone;
2-(4-(2-(4-fluorophenyl)acetyl)morpholin-2-yl)-N-isobutylquinoline-4-carboxamide;
2-(4-((1H-indol-5-yl)methyl)morpholin-2-yl)-N-(2-methoxyethyl)quinoline-4-carboxamide;
N-(2-hydroxyethyl)-2-(4-(quinolin-6-ylmethyl)morpholin-2-yl)quinoline-4-carboxamide;
phenyl(2-(5-(3-(trifluoromethyl)benzyl)pyridin-2-yl)morpholino)methanone;
1-(2-(5-(2-fluorobenzyl)pyridin-2-yl)morpholino)-3-(1H-indol-3-yl)propan-1-one;
2-(5-(4-methoxybenzyl)pyridin-2-yl)-4-(quinolin-4-ylmethyl)morpholine;
1-(2-(6-(2-fluorobenzyl)pyridin-2-yl)morpholino)-2-(3-fluorophenyl)-2-methylpropan-1-one;
N-isobutyl-2-(4-(2-phenylacetyl)morpholin-2-yl)quinoline-4-carboxamide;
1-(2-(4-(3-fluorobenzyl)-6-methylpyridin-2-yl)morpholino)-2-(4-fluorophenyl)ethan-1-one;
3-(6-(4-(4,6-dimethylpyrimidin-2-yl)morpholin-2-yl)pyridin-3-yl)propenamide;
4-((1,5-dimethyl-1H-pyrazol-4-yl)methyl)-2-(5-(pyrimidin-5-yl)pyridin-2-yl)morpholine;
(2-(6-(2-fluorobenzyl)pyridin-2-yl)morpholino)(2-(methylamino)pyridin-4-yl)methanone;
N-(6-(4-(quinolin-5-ylmethyl)morpholin-2-yl)pyridin-2-yl)pyrazin-2-amine;
N-(6-(4-((6-methoxy-1-methyl-1H-indol-3-yl)methyl)morpholin-2-yl)pyridin-2-yl)-4-methylthiazol-2-amine;
N-(6-(4-((2-(dimethylamino)pyridin-3-yl)methyl)morpholin-2-yl)pyridin-2-yl)-4-methylthiazol-2-amine;
4-methyl-N-(6-(4-(3-methylbenzyl)morpholin-2-yl)pyridin-2-yl)thiazol-2-amine;
2-(6-(4-methoxyphenyl)pyridin-2-yl)-4-(quinolin-5-ylmethyl)morpholine;
2-(6-(2-fluorobenzyl)pyridin-2-yl)-4-(quinolin-5-ylmethyl)morpholine;
2-(1H-benzo[d]imidazol-1-yl)-1-(2-(4-(2-fluorobenzyl)-6-methylpyridin-2-yl)morpholino)ethan-1-one;
morpholino(2-(4-(3-phenylpropyl)morpholin-2-yl)quinolin-4-yl)methanone;
(2-(6-(3-methoxybenzyl)pyridin-2-yl)morpholino)(quinolin-8-yl)methanone;
3-(3-((2-(6-(3-fluorophenyl)pyridin-2-yl)morpholino)methyl)-1H-indol-1-yl)propan-1-ol;
3-(1H-benzo[d]imidazol-1-yl)-1-(2-(6-(4-fluorobenzyl)pyridin-2-yl)morpholino)propan-1-one;
2-(1H-indol-3-yl)-1-(2-(6-(3-methoxybenzyl)pyridin-2-yl)morpholino)ethan-1-one;
N-(6-(4-(quinolin-4-ylmethyl)morpholin-2-yl)pyridin-2-yl)pyrazin-2-amine;
5-fluoro-N-(6-(4-((6-methoxy-1-methyl-1H-indol-3-yl)methyl)morpholin-2-yl)pyridin-2-yl)pyridin-2-amine;
N-benzyl-N-isopropyl-2-(2-(6-(pyridin-2-ylamino)pyridin-2-yl)morpholino)acetamide;
(2-(6-methyl-4-((6-methylpyridin-2-yl)amino)pyridin-2-yl)morpholino)(quinolin-8-yl)methanone;
1-(2-(6-(2-fluorobenzyl)pyridin-2-yl)morpholino)-3-phenylpropan-1-one;
1-(3-(4-(2-fluorobenzyl)-6-methylpyridin-2-yl)piperidin-1-yl)-3-(3-methyl-1H-pyrazol-1-yl)propan-1-one;
2-phenyl-1-(2-(6-(2-(trifluoromethyl)benzyl)pyridin-2-yl)morpholino)ethan-1-one;
N-benzyl-N-isopropyl-2-(2-(6-(pyrimidin-2-ylamino)pyridin-2-yl)morpholino)acetamide;
2′-(4-(2,4-dimethylpyrimidine-5-carbonyl)morpholin-2-yl)-N,6′-dimethyl-[3,4′-bipyridine]-6-carboxamide;
(2-(4-isonicotinoylmorpholin-2-yl)quinolin-4-yl)(morpholino)methanone;
pyridin-4-yl(2-(5-(3-(trifluoromethyl)benzyl)pyridin-2-yl)morpholino)methanone;
2-phenyl-1-(2-(5-(3-(trifluoromethyl)benzyl)pyridin-2-yl)morpholino)ethan-1-one;
(4-fluorophenyl)(2-(5-(3-(trifluoromethyl)benzyl)pyridin-2-yl)morpholino)methanone;
5-fluoro-N-(6-(4-((1-methyl-1H-indol-3-yl)methyl)morpholin-2-yl)pyridin-2-yl)pyridin-2-amine;
N-(6-(4-((1-ethyl-6-fluoro-1H-indol-3-yl)methyl)morpholin-2-yl)pyridin-2-yl)pyrazin-2-amine;
3-(1H-indazol-1-yl)-1-(2-(6-((4-methylthiazol-2-yl)amino)pyridin-2-yl)morpholino)propan-1-one;
N-(6-(4-((5-fluoro-1-methyl-1H-indol-3-yl)methyl)morpholin-2-yl)pyridin-2-yl)-4-methylthiazol-2-amine;
5-methyl-N-(2-methyl-6-(4-(quinolin-6-ylmethyl)morpholin-2-yl)pyridin-4-yl)thiazol-2-amine;
4-((1,5-dimethyl-1H-pyrazol-4-yl)methyl)-2-(6-(4-fluorobenzyl)pyridin-2-yl)morpholine;
1-(2-(6-benzylpyridin-2-yl)morpholino)-3-(1H-tetrazol-1-yl)propan-1-one;
4-((1,3-dimethyl-1H-pyrazol-4-yl)methyl)-2-(6-(2-fluorobenzyl)pyridin-2-yl)morpholine;
1-(2-(6-benzylpyridin-2-yl)morpholino)-3-(4-fluorophenyl)propan-1-one;
3-(3,5-dimethyl-1H-1,2,4-triazol-1-yl)-1-(2-(4-(4-fluorobenzyl)-6-methylpyridin-2-yl)morpholino)propan-1-one;
1-(2-(6-methyl-4-(3-(trifluoromethyl)benzyl)pyridin-2-yl)morpholino)-2-(1H-pyrazol-1-yl)ethan-1-one;
1-(2-(6-benzylpyridin-2-yl)morpholino)-2-(4-fluorophenyl)ethan-1-one;
2-(3,5-dimethyl-1H-pyrazol-1-yl)-1-(2-(6-methyl-4-(2-methylbenzyl)pyridin-2-yl)morpholino)ethan-1-one;
(6-aminopyridin-3-yl)(2-(4-(2-fluorobenzyl)-6-methylpyridin-2-yl)morpholino)methanone;
1-(2-(6-(3-chlorobenzyl)pyridin-2-yl)morpholino)-2-(4-fluorophenyl)ethan-1-one;
4-((1,5-dimethyl-1H-pyrazol-4-yl)methyl)-2-(6-(2-methylbenzyl)pyridin-2-yl)morpholine;
2-(4-benzyl-6-methylpyridin-2-yl)-4-((1,3-dimethyl-1H-pyrazol-4-yl)methyl)morpholine;
N,N-dimethyl-5-((2-(6-methyl-4-(4-methylbenzyl)pyridin-2-yl)morpholino)methyl)pyrimidin-2-amine;
N,N-dimethyl-5-((2-(6-methyl-4-(4-methylbenzyl)pyridin-2-yl)morpholino)methyl)pyrimidin-2-amine;
(1,2-dimethyl-1H-benzo[d]imidazol-5-yl)(2-(6-methyl-4-(pyridin-2-ylamino)pyridin-2-yl)morpholino)methanone;
3-(3,5-dimethyl-1H-1,2,4-triazol-1-yl)-1-(2-(6-(4-fluorobenzyl)pyridin-2-yl)morpholino)propan-1-one;
2-(4-(4-fluorobenzyl)-6-methylpyridin-2-yl)-4-((1-methyl-1H-pyrazol-4-yl)methyl)morpholine;
1-(2-(6-(2,6-difluorobenzyl)pyridin-2-yl)morpholino)-2-morpholinoethan-1-one;
(2-(6-(2,6-difluorobenzyl)pyridin-2-yl)morpholino)(2-(methylamino)pyridin-4-yl)methanone;
1-(2-(5-(3-chlorobenzyl)pyridin-2-yl)morpholino)-3-(3,5-dimethyl-1H-1,2,4-triazol-1-yl)propan-1-one;
3-((2-(6-(4-methoxyphenyl)pyridin-2-yl)morpholino)methyl)-N,N-dimethylpyridin-2-amine;
1-(2-(6-benzylpyridin-2-yl)morpholino)-3-phenylpropan-1-one;
(2-(dimethylamino)-4-methylpyrimidin-5-yl)(2-(6-methyl-4-(4-methylbenzyl)pyridin-2-yl)morpholino)methanone;
(2-(6-methyl-4-(3-(trifluoromethyl)benzyl)pyridin-2-yl)morpholino)(pyridin-3-yl)methanone;
(4-(ethoxymethyl)phenyl)(2-(6-methyl-4-((3-methylpyridin-2-yl)amino)pyridin-2-yl)morpholino)methanone;
5-((2-(6-(2-chlorobenzyl)pyridin-2-yl)morpholino)methyl)-N,N-dimethylpyrimidin-2-amine;
2-(1-ethylpiperidin-4-yl)-1-(2-(3′-methoxy-[1,1′-biphenyl]-4-yl)morpholino)ethan-1-one;
2-(1-ethylpiperidin-4-yl)-1-(3-(5-(3-methoxyphenyl)pyridin-2-yl)piperidin-1-yl)ethan-1-one;
2-(1-ethylpiperidin-4-yl)-1-(1-(5-(3-methoxyphenyl)pyridin-2-yl)piperidin-3-yl)ethan-1-one;
1-ethylpiperidin-4-yl 2-(5-(3-methoxyphenyl)pyridin-2-yl)morpholine-4-carboxylate;
4-(2-(1-ethylpiperidin-4-yl)ethyl)-2-(5-(3-methoxyphenyl)pyridin-2-yl)morpholine;
2-(1-ethylpiperidin-4-yl)-1-(2-(5-(4-fluorobenzyl)pyridin-2-yl)morpholino)ethan-1-one;
1-(2-(5-(4-fluorobenzyl)pyrazin-2-yl)morpholino)-3-(1H-indazol-1-yl)propan-1-one;
1-(2-(5-(4-fluorobenzyl)pyrimidin-2-yl)morpholino)-3-(1H-indazol-1-yl)propan-1-one;
3-(1H-indazol-1-yl)-1-(2-(5-(3-methoxyphenyl)pyridin-2-yl)morpholino)propan-1-one;
1-(2-(5-(4-fluorophenoxy)pyridin-2-yl)morpholino)-3-(1H-indazol-1-yl)propan-1-one;
2-(1-ethylpiperidin-4-yl)-1-(2-(6-(3-methoxyphenyl)pyridin-3-yl)morpholino)ethan-1-one;
2-(3-ethyl-3-azabicyclo[3.1.0]hexan-6-yl)-1-(2-(5-(3-methoxyphenyl)pyridin-2-yl)morpholino)ethan-1-one;
1-(2-(4-(4-fluorobenzyl)phenyl)morpholino)-3-(1H-pyrazol-1-yl)propan-1-one;
4-(3-(1H-indazol-1-yl)propyl)-2-(4-(4-fluorobenzyl)phenyl)morpholine;
2-(1-ethylpiperidin-4-yl)-1-(2-(5-phenylpyridin-2-yl)morpholino)ethan-1-one; and
2-(4-ethylpiperazin-1-yl)-1-(2-(5-(3-methoxyphenyl)pyridin-2-yl)morpholino)ethan-1-one.

25-34. (canceled)
